(19) United States
(12) Patent Application Publication
(10) Pub. No.: US 2010/0124547 A1
(43) Pub. Date: May 20, 2010

(54) COMPOSITIONS AND METHODS FOR INHIBITING EXPRESSION OF FACTOR VII GENES

(76) Inventors: Birgit Bramlage, Kulmbach (DE); Rainer Constien, Kulmbach (DE); Jacques Himber, Guebwiller (FR); Markus Hossbach, Kulmbach (DE); Pamela Tan, Kulmbach (DE); Hans-Peter Vornlocher, Bayreuth (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/616,828

(22) Filed: Nov. 12, 2009

(30) Foreign Application Priority Data
Nov. 17, 2008 (EP) 08169301.2

(51) Int. Cl. A61K 35/12; C12N 5/10; C12N 5/02; C12N 15/63; A61K 31/7088; C07H 21/02; A01K 67/00

(52) U.S. Cl. 424/93.7; 435/325; 435/375; 435/320.1; 514/44 R; 514/44 A; 536/24.5; 800/13

(57) ABSTRACT

The invention relates to a double-stranded ribonucleic acid (dsRNA) for inhibiting the expression of a Factor VII gene. The invention also relates to a pharmaceutical composition comprising the dsRNA or nucleic acid molecules or vectors encoding the same together with a pharmaceutically acceptable carrier; methods for treating diseases caused by the expression of a Factor VII gene using said pharmaceutical composition; and methods for inhibiting the expression of Factor VII in a cell.

### PRIORITY TO RELATED APPLICATION(S)

This application claims the benefit of European Patent Application No. 08169301.2 filed Nov. 17, 2008, which is hereby incorporated by reference in its entirety.

### BACKGROUND OF THE INVENTION

This invention relates to double-stranded ribonucleic acids (dsRNAs), and their use in mediating RNA interference to inhibit the expression of the factor VII gene, in particular in the inhibition of the factor VII zymogen expression in the liver and subsequently in lowering the factor VII zymogen plasma levels. Furthermore, the use of said dsRNAs to treat/prevent a wide range of thromboembolic diseases/disorders which are associated with the activation of clotting factors VIIa, IXa, Xa, XIIa, thrombin, like arterial and venous thrombosis, inflammation, arteriosclerosis and cancer is part of the invention.

Factor VII (FVII) is a vitamin K-dependent glycoprotein that participates in the initiation of the extrinsic pathway of blood coagulation. FVII is synthesized in the liver and circulates mainly in plasma as an inactive single-chain zymogen. Upon binding to tissue factor (TF) exposed by vascular injury, FVII is cleaved to its two-chain active form (FVIIa) by cleavage of a single peptide bond resulting in a light chain of 20-kDa and a heavy chain of 30-kDa. The light chain of FVIIa comprises two epidermal growth factor-like (EGF-1, EGF-2) domains and a γ-carboxyglutamic acid (Gla) domain which allows the binding of calcium causing a conformational change in the molecule, exposing novel epitopes and facilitating its subsequent binding to TF. The heavy chain contains the catalytic domain which is structurally homologous to the other serine proteases of the coagulation. The TF:FVIIa complex in turn activate FIX and FX by limited proteolytic cleavage leading to thrombin formation and finally to a fibrin clot.

The human FVII gene is expressed in hepatocytes but the steady state level of FVII mRNA is very low. The complete sequence of human FVII has been inferred from a full-length cDNA clone (Hagen F. S., et al., Proc. Natl. Acad. Sci. USA (1986) 83:2412-2416). Elevated levels of FVII have been associated with independent risk factors for the development of cardiovascular disease. In hypercholesterolemic patients FVII level was independently correlated with proinflammatory variables such as C-reactive protein (CRP) or cytokines (IL-6). However not all studies have confirmed FVII as an independent risk factor in coronary heart disease (Lowe G. D. O. et al., Arterioscler. Thromb. Vasc. Biol. (2004) 24:1529-1534).

The TF:FVIIa complex plays a critical role in the complex crosstalk between coagulation and inflammatory responses. In addition to its well-established role in coagulation TF:FVIIa complex also induces intracellular changes such as signal transduction which affects cellular processes like inflammation, angiogenesis and the pathophysiology of cancer and atherosclerosis.

Proof of concept experiments in animal models have demonstrated that a specific inhibition of FVIIa or a reduction of FVII zymogen level in plasma results in antithrombotic and anti-inflammatory effects without enhancing bleeding propensity (Xu H., et al., J. Pathol. (2006) 210:488-496). In sepsis models, inhibition of endotoxin-induced coagulation activation, reduction of the expression of inflammatory mediators interleukin-6 (Il-6), IL-8 and prevention of mortality was observed in monkeys treated with either an active site-inactivated FVIIa (Taylor F. et al., Blood. (1998) 91:1609-1615) or a monoclonal Fab fragment against FVIIIVIIa (Biemond B. J. et al., Thromb. Haemost. (1995) 73:223-230). Active site-inactivated FVIIa showed also powerful anti-inflammatory properties in experimental acute pancreatitis (Andersson E. et al., Scand. J. Gastroenterology (2007) 42: 765-770), preventing tissue infiltration of neutrophils in lung, ileum and colon and reducing the inflammatory markers such as IL-6 and macrophage inflammatory protein-2 (MIP-2).

Moreover, intra-articular injection of TF:FVIIa complex in mice induces monocytes infiltration into synovial tissue followed by cartilage and bone destruction. Arthritis severity was significantly reduced in TF mutant mice indicating that TF/FVII complexes, frequently found intra-articularly in joints of rheumatoid arthritis patients, is an important component in both induction and progression of chronic destructive arthritis. (Yang Y. H. et al., Am. J. Pathol. (2004) 164:109-117).

Blocking the TF:FVIIa complex by either anti-TF monoclonal antibody (Mueller B. M. et al., Proc. Nall. Acad. Sci. USA (1992) 89:11832-11836), tissue factor pathway inhibitor (Amirkhosravi A. et al., Semin. Thromb. Hemost. (2007) 33:643-652) or knocking down the TF expression by specific TF siRNA inhibit experimental lung metastasis (Amarzguioui M. et al., Clin. Cancer Res. (2006) 12:4055-4061), suggesting that the TF:FVIIa complex is also involved in the promotion of tumor growth and metastasis and further suggest that inhibition of the TF:FVIIa complex is a clinical viable strategy for the treatment of cancer.

Despite significant advances in the treatment of thrombotic and inflammatory disorders, current understanding of e.g. coronary artery disease, atherosclerosis, rheumatoid arthritis, proliferative disorders like cancers/metastases, suggest that a therapeutically active and safe substance with both anti-thrombotic and anti-inflammatory properties is an improvement over standard therapy. Double-stranded RNA molecules (dsRNA) have been shown to block gene expression in a highly conserved regulatory mechanism known as RNA interference (RNAi).

### SUMMARY OF THE INVENTION

The invention provides double-stranded ribonucleic acid molecules (dsRNAs) able to selectively and efficiently decrease the expression of FVII. The use of FVII RNAi provides a method for the therapeutic and/or prophylactic treatment of diseases/disorders which are associated with the formation of FVIIa, TF-FVIIa complex, clotting factors like IXa, Xa, XIIa and thrombin, inflammation factors like cytokines and C-reactive protein (CRP), activated directly or indirectly by FVIIa and TF. Particular disease/disorder states include the therapeutic and/or prophylactic treatment of arterial and venous thrombosis, deep venous thrombosis, unstable angina pectoris, acute coronary syndrome, myocardial infarction, stroke due to atrial fibrillation, pulmonary embolism, cerebral embolism, kidney embolism, critical limb ischemia, acute limb ischemia, disseminated intravascular coagulation (caused e.g. by bacteria, viral diseases, cancer, sepsis, multiple trauma), gangrene, Sickle cell disease, periateritis nodosale, Kawasaki syndrome, Buerger disease, antiphospholipid syndrome, inflammatory responses including but not limited to acute or chronic atherosclerosis, rheumatoid arthritis, proliferative disorders like cancer/metastases, pancreatitis, which method comprises administration of dsRNA targeting FVII to a human being or animal. The compounds of this invention can also be used in prevention of thrombosis when blood is in contact with medical devices inside the body (e.g. mechanical and biological prosthetic cardiac valves, vascular stents, vascular catheter, vascular grafts) or outside the body (e.g. haemodialysis, heart-lung machine).

### DETAILED DESCRIPTION OF THE INVENTION

The invention provides double-stranded ribonucleic acid molecules (dsRNAs) able to selectively and efficiently decrease the expression of FVII in hepatocytes by silencing the FVII gene(s), thereby decreasing the level of FVII protein synthesized in the liver and finally reducing the FVII activity in plasma. In one preferred embodiment the described dsRNA molecule is capable of inhibiting the expression of a FVII gene by at least 70%. The invention also provides compositions and methods for specifically targeting the liver with FVII dsRNA, for treating pathological conditions and diseases caused by the expression of the FVII gene including those described above.

In one embodiment, the invention provides double-stranded ribonucleic acid (dsRNA) molecules for inhibiting the expression of a Factor VII, in particular the expression of the mammalian or human Factor VII gene. The dsRNA comprises at least two sequences that are complementary to each other. The dsRNA comprises a sense strand comprising a first sequence and an antisense strand may comprise a second sequence, see also provision of specific dsRNA pairs in the appended tables 1, 4, 6 and 7. In one embodiment the sense strand comprises a sequence which has an identity of at least 90% to at least a portion of an mRNA encoding FVII. Said sequence is located in a region of complementarity of the sense strand to the antisense strand. In one preferred embodiment the dsRNA targets particularly the human Factor VII gene, in yet another preferred embodiment the dsRNA targets the guinea pig (Cavia porcellus) or rat (Rattus norvegicus) Factor VII gene.

In one embodiment, the antisense strand comprises a nucleotide sequence which is substantially complementary to at least part of an mRNA encoding said Factor VII gene, and the region of complementarity is most preferably less than 30 nucleotides in length. Furthermore, it is preferred that the length of the herein described inventive ds molecules (duplex length) is in the range of about 16 to 30 nucleotides, in particular in the range of about 18 to 28 nucleotides. Particularly useful in context of this invention are duplex lengths of about 19, 20, 21, 22, 23 or 24 nucleotides. Most preferred are duplex stretches of 19, 21 or 23 nucleotides. The dsRNA, upon contacting with a cell expressing a Factor VII gene, inhibits the expression of a Factor VII gene in vitro by at least 70%.

Selected dsRNA molecules are provided in the appended tables 6 and 7, with preferred dsRNA molecules comprising nucleotides 1-19 of SEQ ID Nos: 413, 414, 415, 416, 417, 418, 419, 420, 421, 422, 423, 424, 425, 426, 427, 428, 429, 430, 431, 432, 433, 434, 435, 436, 437 and 438.

In one embodiment said dsRNA molecules comprise an antisense strand with a 3′ overhang of 1-5 nucleotides length, preferably of 1-2 nucleotides length. Preferably said overhang of the antisense strand comprises uracil or nucleotides which are at least 90% complementary to the mRNA encoding Factor VII.

In another preferred embodiment, said dsRNA molecules comprise a sense strand with a 3′ overhang of 1-5 nucleotides length, preferably of 1-2 nucleotides length. Preferably said overhang of the sense strand comprises uracil or nucleotides which are at least 90% identical to the mRNA encoding Factor VII.

In another preferred embodiment, said dsRNA molecules comprise a sense strand with a 3′ overhang of 1-5 nucleotides length, preferably of 1-2 nucleotides length, and an antisense strand with a 3′ overhang of 1-5 nucleotides length, preferably of 1-2 nucleotides length. Preferably said overhang of the sense strand comprises uracil or nucleotides which are at least 90% identical to the mRNA encoding Factor VII and said overhang of the antisense strand comprises uracil or nucleotides which are at least 90% complementary to the mRNA encoding Factor VII.

In preferred dsRNA molecules, inter alia and preferably, the sense strand is selected from the group consisting of the nucleic acid sequences depicted in SEQ ID Nos: 413, 415, 417, 419, 421, 423, 425, 427, 429, 431, 433, 435, and 437 and the antisense strand is selected from the from the group consisting of the nucleic acid sequences depicted in SEQ ID Nos: 414, 416, 418, 420, 422, 424, 426, 428, 430, 432, 434, 436 and 438. Accordingly, the inventive dsRNA molecule may, inter alia, comprise the sequence pairs selected from the group consisting of SEQ ID Nos: 413/414, 415/416, 417/418, 419/420, 421/422, 423/424, 425/426, 427/428, 429/430, 431/432, 433/434, 435/436 and 437/438. In context of specific dsRNA molecules provided herein, pairs of SEQ ID Nos relate to corresponding sense and antisense strands sequences (5′ to 3′) as also shown in appended tables.

Also modified dsRNA molecules are provided herein and are in particular disclosed in appended tables 1 and 4, providing illustrative examples of modified dsRNA molecules of the present invention.

Tables 2 and 3 provide for selective biological, clinically and pharmaceutical relevant parameters of certain dsRNA molecules of this invention.

As pointed out herein above, Table 1 provides for illustrative examples of modified dsRNAs of this invention (whereby the corresponding sense strand and antisense strand is provided in this table). Yet, the illustrative modifications of these constituents of the inventive dsRNAs are provided herein as examples of modifications. Also further modifications of these dsRNAs (and their constituents) are comprised as one embodiment of this invention. Corresponding examples are provided in the more detailed description of this invention.

Appended Tables 4 and 7 also provide for further siRNA molecules/dsRNA useful in context of this invention, whereby Table 4 provides for certain biological and/or clinically relevant surprising features of the modified siRNA molecules/dsRNA molecules of this invention as shown in Table 7. These RNA molecules comprise illustrative nucleotide modifications.

Most preferred dsRNA molecules are provided in the appended tables 1 and 4 and, inter alia and preferably, wherein the sense strand is selected from the group consisting of the nucleic acid sequences depicted in SEQ ID Nos: 1, 3, 5, 7, 9, 11, 13, 15, 17, 19, 21, 23 and 25 and the antisense strand is selected from the from the group consisting of the nucleic acid sequences depicted in SEQ ID Nos: 2, 4, 6, 8, 10, 12, 14, 16, 18, 20, 22, 24 and 26. Accordingly, the inventive dsRNA molecule may, inter alia, comprise the sequence pairs selected from the group consisting of SEQ ID Nos: 1/2, 3/4, 5/6, 7/8, 9/10, 11/12, 13/14, 15/16, 17/18, 19/20, 21/22, 23/24 and 25/26. Most preferred dsRNA molecules comprise sequence pairs 19/20 and 11/12. In context of specific dsRNA molecules provided herein, pairs of SEQ ID Nos relate to corresponding sense and antisense strands sequences (5′ to 3′) as also shown in appended and included tables.

In one embodiment the dsRNA molecules of the invention comprises of an sense and antisense strand wherein at least one of said strands has a half-life of at least 24 hours. In another embodiment the dsRNA molecules of the invention are non-immunostimulatory, e.g. do not stimulate INF-á and TNF-á in vitro.

The dsRNA molecules of the invention may be comprised of naturally occurring nucleotides or may be comprised of at least one modified nucleotide, such as a 2′-O-methyl modified nucleotide, a nucleotide comprising a 5′-phosphorothioate group, and a terminal nucleotide linked to a cholesteryl derivative or dodecanoic acid bisdecylamide group. 2′ modified nucleotides may have the additional advantage that certain immunostimulatory factors or cytokines are suppressed when the inventive dsRNA molecules are employed in vivo, for example in a medical setting. Alternatively and non-limiting, the modified nucleotide may be chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide. In one preferred embodiment the dsRNA molecules comprises at least one of the following modified nucleotides: a 2′-O-methyl modified nucleotide, a nucleotide comprising a 5′-phosphorothioate group and a deoxythymidine. Preferred dsRNA molecules comprising modified nucleotides are given in tables 1 and 4.

The invention also provides for cells comprising at least one of the dsRNAs of the invention. The cell is preferably a mammalian cell, such as a human cell. Furthermore, also tissues and/or non-human organisms comprising the herein defined dsRNA molecules are comprised in this invention, whereby said non-human organism is particularly useful for research purposes or as research tool, for example also in drug testing.

Furthermore, the invention relates to a method for inhibiting the expression of a FVII gene, in particular a mammalian or human FVII gene, in a cell, tissue or organism comprising the following steps:

(a) introducing into the cell, tissue or organism a double-stranded ribonucleic acid (dsRNA) as defined herein;
(b) maintaining said cell, tissue or organism produced in step (a) for a time sufficient to obtain degradation of the mRNA transcript of a FVII gene, thereby inhibiting expression of a FVII gene in a given cell.

The invention also relates to pharmaceutical compositions comprising the inventive dsRNAs of this invention. These pharmaceutical compositions are particularly useful in the inhibition of the expression of a FVII gene in a cell, a tissue or an organism. The pharmaceutical composition comprising one or more of the dsRNA of the invention may also comprise (a) pharmaceutically acceptable carrier(s), diluent(s) and/or excipient(s).

In another embodiment, the invention provides methods for treating, preventing or managing thrombotic disorders which are associated with the activation of clotting factors, inflammations or proliferative disorders, said method comprising administering to a subject in need of such treatment, prevention or management a therapeutically or prophylactically effective amount of one or more of the dsRNAs of the invention. Preferably, said subject is a mammal, most preferably a human patient.

In one embodiment, the invention provides a method for treating a subject having a pathological condition mediated by the expression of a Factor VII gene. Such conditions comprise disorders, such as thromboembolic disorders, undesired inflammation events or proliferative disorders and those described above. In this embodiment, the dsRNA acts as a therapeutic agent for controlling the expression of a Factor VII gene. The method comprises administering a pharmaceutical composition of the invention to the patient (e.g., human), such that expression of a Factor VII gene is silenced. Because of their high specificity, the dsRNAs of the invention specifically target mRNAs of a Factor VII gene. In one preferred embodiment the described dsRNAs specifically decrease FVII mRNA levels and do not directly affect the expression and/or mRNA levels of off-target genes in the cell.

In one preferred embodiment the described dsRNA decrease Factor VII mRNA levels in the liver by at least 80% in vivo, and decrease Factor VII zymogen levels in the plasma by at least 95% in vivo. In another embodiment the described dsRNAs prolong prothrombin time and inhibit thrombin generation and thrombus formation in vivo. In yet another preferred embodiment these antithrombotic effects mediated by the described dsRNA molecules are associated with decreased in vivo plasma FVII levels and decreased in vivo liver FVII mRNA levels.

In one embodiment the described dsRNA molecules increase the blood clotting time in vivo at least twofold.

Particularly useful with respect to therapeutic dsRNAs is the set of dsRNAs targeting guinea pig Factor VII which can be used to estimate toxicity, therapeutic efficacy and effective dosages and in vivo half-lives for the individual dsRNAs in a guinea pig or cell culture model.

In another embodiment, the invention provides vectors for inhibiting the expression of a Factor VII gene in a cell, in particular Factor VII gene comprising a regulatory sequence operable linked to a nucleotide sequence that encodes at least one strand of one of the dsRNA of the invention.

In another embodiment, the invention provides a cell comprising a vector for inhibiting the expression of a Factor VII gene in a cell. Said vector comprises a regulatory sequence operable linked to a nucleotide sequence that encodes at least one strand of one of the dsRNA of the invention. Yet, it is preferred that said vector comprises, besides said regulatory sequence a sequence that encodes at least one “sense strand” of the inventive dsRNA and at least one “anti sense strand” of said dsRNA. It is also envisaged that the claimed cell comprises two or more vectors comprising, besides said regulatory sequences, the herein defined sequence(s) that encode(s) at least one strand of one of the dsRNA of the invention.

In one embodiment, the method comprises administering a composition comprising a dsRNA, wherein the dsRNA comprises a nucleotide sequence which is complementary to at least a part of an RNA transcript of a Factor VII gene of the mammal to be treated. As pointed out above, also vectors and cells comprising nucleic acid molecules that encode for at least one strand of the herein defined dsRNA molecules can be used as pharmaceutical compositions and may, therefore, also be employed in the herein disclosed methods of treating a subject in need of medical intervention. It is also of note that these embodiments relating to pharmaceutical compositions and to corresponding methods of treating a (human) subject also relate to approaches like gene therapy approaches. Factor VII specific dsRNA molecules as provided herein or nucleic acid molecules encoding individual strands of these inventive dsRNA molecules may also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

In another aspect of the invention, Factor VII specific dsRNA molecules that modulate Factor VII gene expression activity are expressed from transcription units inserted into DNA or RNA vectors (see, e.g., Skillern, A., et al., International PCT Publication No. WO 00/22113). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann, et al., Proc. Natl. Acad. Sci. USA (1995) 92:1292).

The individual strands of a dsRNA can be transcribed by promoters on two separate expression vectors and co-transfected into a target cell. Alternatively each individual strand of the dsRNA can be transcribed by promoters both of which are located on the same expression plasmid. In a preferred embodiment, a dsRNA is expressed as an inverted repeat joined by a linker polynucleotide sequence such that the dsRNA has a stem and loop structure.

The recombinant dsRNA expression vectors are preferably DNA plasmids or viral vectors. dsRNA expressing viral vectors can be constructed based on, but not limited to, adeno-associated virus (for a review, see Muzyczka, et al., Curr. Topics Micro. Immunol. (1992) 158:97-129)); adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Danos and Mulligan, Proc. Natl. Acad. Sci. USA (1998) 85:6460-6464). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

The promoter driving dsRNA expression in either a DNA plasmid or viral vector of the invention may be a eukaryotic RNA polymerase I (e.g. ribosomal RNA promoter), RNA polymerase II (e.g. CMV early promoter or actin promoter or U1 snRNA promoter) or preferably RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g. the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Preferably, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single A Factor VII gene or multiple A Factor VII genes over a period of a week or more are also contemplated by the invention. Successful introduction of the vectors of the invention into host cells can be monitored using various known methods. For example, transient transfection can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The following detailed description discloses how to make and use the dsRNA and compositions containing dsRNA to inhibit the expression of a target Factor VII gene, as well as compositions and methods for treating diseases and disorders caused by the expression of said Factor VII gene.

### DEFINITIONS

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A”, “U” and “T” or “dT” respectively, each generally stand for a nucleotide that contains guanine, cytosine, adenine, uracil and deoxythymidine as a base, respectively. However, the term “ribonucleotide” or “nucleotide” can also refer to a modified nucleotide, as further detailed below, or a surrogate replacement moiety. Sequences comprising such replacement moieties are embodiments of the invention. As detailed below, the herein described dsRNA molecules may also comprise “overhangs”, i.e. unpaired, overhanging nucleotides which are not directly involved in the RNA double helical structure normally formed by the herein defined pair of “sense strand” and “anti sense strand”. Often, such an overhanging stretch comprises the deoxythymidine nucleotide, in most embodiments, 2 deoxythymidines in the 3′ end. Such overhangs will be described and illustrated below.

The term Factor VII” or “FVII” as used herein relates in particular to the coagulation factor VII also formerly described as “proconvertin” or “serum prothrombin conversion accelerator” and said term relates to the corresponding gene, encoded mRNA, encoded protein/polypeptide as well as functional fragments of the same. The term “Factor VII gene/sequence” does not only relate to (the) wild-type sequence(s) but also to mutations and alterations which may be comprised in said gene/sequence. Accordingly, the present invention is not limited to the specific dsRNA molecules provided herein. The invention also relates to dsRNA molecules that comprise an antisense strand that is at least 85% complementary to the corresponding nucleotide stretch of an RNA transcript of a Factor VII gene that comprises such mutations/alterations.

As used herein, “target sequence” refers to a contiguous portion of the nucleotide sequence of an mRNA molecule formed during the transcription of a Factor VII gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature. However, as detailed herein, such a “strand comprising a sequence” may also comprise modifications, like modified nucleotides.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

Sequences referred to as “fully complementary” comprise base-pairing of the oligonucleotide or polynucleotide comprising the first nucleotide sequence to the oligonucleotide or polynucleotide comprising the second nucleotide sequence over the entire length of the first and second nucleotide sequence.

However, where a first sequence is referred to as “substantially complementary” with respect to a second sequence herein, the two sequences can be fully complementary, or they may form one or more, but preferably not more than 4, 3 or 2 mismatched base pairs upon hybridization.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

The term “double-stranded RNA” or “dsRNA”, as used herein, refers to a ribonucleic acid molecule, or complex of ribonucleic acid molecules, having a duplex structure comprising two anti-parallel and substantially complementary nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop”. Where the two strands are connected covalently by means other than an uninterrupted chain of nucleotides between the 3′-end of one strand and the 5′-end of the respective other strand forming the duplex structure, the connecting structure is referred to as a “linker”. The RNA strands may have the same or a different number of nucleotides. In addition to the duplex structure, a dsRNA may comprise one or more nucleotide overhangs. The nucleotides in said “overhangs” may comprise between 0 and 5 nucleotides, whereby “0” means no additional nucleotide(s) that form(s) an “overhang” and whereas “5” means five additional nucleotides on the individual strands of the dsRNA duplex. These optional “overhangs” are located in the 3′ end of the individual strands. As will be detailed below, also dsRNA molecules which comprise only an “overhang” in one the two strands may be useful and even advantageous in context of this invention. The “overhang” comprises preferably between 0 and 2 nucleotides. Most preferably 2 “dT” (deoxythymidine) nucleotides are found at the 3′ end of both strands of the dsRNA. Accordingly, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence. Where the region of complementarity is not fully complementary to the target sequence, the mismatches are most tolerated in the terminal regions and, if present, are preferably in a terminal region or regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand. “Substantially complementary” means preferably at least 85% of the overlapping nucleotides in sense and antisense strand are complementary.

“Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell”, wherein the cell is part of a living organism. In such instance, introduction into the cell will include the delivery to the organism. For example, for in vivo delivery, dsRNA can be injected into a tissue site or administered systemically. It is, for example envisaged that the dsRNA molecules of this invention be administered to a subject in need of medical intervention. Such an administration may comprise the injection of the dsRNA, the vector or an cell of this invention into a diseased side in said subject, for example into liver tissue/cells or into cancerous tissues/cells, like liver cancer tissue. However, also the injection in close proximity of the diseased tissue is envisaged. In vitro introduction into a cell includes methods known in the art such as electroporation and lipofection.

The terms “silence”, “inhibit the expression of” and “knock down”, in as far as they refer to a Factor VII gene, herein refer to the at least partial suppression of the expression of a Factor VII gene, as manifested by a reduction of the amount of mRNA transcribed from a Factor VII gene which may be isolated from a first cell or group of cells in which a Factor VII gene is transcribed and which has or have been treated such that the expression of a Factor VII gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to the Factor VII gene transcription, e.g. the amount of protein encoded by a Factor VII gene which is secreted by a cell, or the number of cells displaying a certain phenotype.

As illustrated in the appended examples and in the appended tables provided herein, the inventive dsRNA molecules are capable of inhibiting the expression of a human Factor VII by at least about 70% in vitro assays, i.e. in vitro. In another embodiment the inventive dsRNA molecules are capable of inhibiting the expression of a guinea pig Factor VII by at least 70%, which also leads to a significant antithrombotic effect in vivo. The person skilled in the art can readily determine such an inhibition rate and related effects, in particular in light of the assays provided herein. Particular preferred dsRNAs are provided, for example in appended Table 1, in particular in rank 1 to 13 (sense strand and antisense strand sequences provided therein in 5′ to 3′ orientation).

The term “off target” as used herein refers to all non-target mRNAs of the transcriptome that are predicted by in silico methods to hybridize to the described dsRNAs based on sequence complementarity. The dsRNAs of the present invention preferably do specifically inhibit the expression of Factor VII, i.e. do not inhibit the expression of any off-target.

The term “half-life” as used herein is a measure of stability of a compound or molecule and can be assessed by methods known to a person skilled in the art, especially in light of the assays provided herein.

The term “non-immunostimulatory” as used herein refers to the absence of any induction of a immune response by the invented dsRNA molecules. Methods to determine immune responses are well known to a person skilled in the art, for example by assessing the release of cytokines, as described in the examples section.

The terms “treat”, “treatment”, and the like, mean in context of this invention to relief from or alleviation of a disorder related to Factor VII expression, like thromboembolic disorders/diseases, inflammations or proliferative disorders.

As used herein, a “pharmaceutical composition” comprises a pharmacologically effective amount of a dsRNA and a pharmaceutically acceptable carrier. However, such a “pharmaceutical composition” may also comprise individual strands of such a dsRNA molecule or the herein described vector(s) comprising a regulatory sequence operably linked to a nucleotide sequence that encodes at least one strand of a sense or an antisense strand comprised in the dsRNAs of this invention. It is also envisaged that cells, tissues or isolated organs that express or comprise the herein defined dsRNAs may be used as “pharmaceutical compositions”. As used herein, “pharmacologically effective amount,” “therapeutically effective amount” or simply “effective amount” refers to that amount of an RNA effective to produce the intended pharmacological, therapeutic or preventive result.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives as known to persons skilled in the art.

It is in particular envisaged that the pharmaceutically acceptable carrier allows for the systemic administration of the dsRNAs, vectors or cells of this invention. Whereas also the enteric administration is envisaged the parenteral administration and also transdermal or transmucosal (e.g. insufflation, buccal, vaginal, anal) administration as well was inhalation of the drug are feasible ways of administering to a patient in need of medical intervention the compounds of this invention. When parenteral administration is employed, this can comprise the direct injection of the compounds of this invention into the diseased tissue or at least in close proximity. However, also intravenous, intraarterial, subcutaneous, intramuscular, intraperitoneal, intradermal, intrathecal and other administrations of the compounds of this invention are within the skill of the artisan, for example the attending physician.

For intramuscular, subcutaneous and intravenous use, the pharmaceutical compositions of the invention will generally be provided in sterile aqueous solutions or suspensions, buffered to an appropriate pH and isotonicity. In a preferred embodiment, the carrier consists exclusively of an aqueous buffer. In this context, “exclusively” means no auxiliary agents or encapsulating substances are present which might affect or mediate uptake of dsRNA in the cells that express a Factor VII gene. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate. The pharmaceutical compositions useful according to the invention also include encapsulated formulations to protect the dsRNA against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in PCT publication WO 91/06309 which is incorporated by reference herein.

As used herein, a “transformed cell” is a cell into which at least one vector has been introduced from which a dsRNA molecule or at least one strand of such a dsRNA molecule may be expressed. Such a vector is preferably a vector comprising a regulatory sequence operably linked to nucleotide sequence that encodes at least one of a sense strand or an antisense strand comprised in the dsRNAs of this invention.

It can be reasonably expected that shorter dsRNAs comprising one of the sequences of Table 1 and 4 minus only a few nucleotides on one or both ends may be similarly effective as compared to the dsRNAs described above. As pointed out above, in most embodiments of this invention, the dsRNA molecules provided herein comprise a duplex length (i.e. without “overhangs”) of about 16 to about 30 nucleotides. Particular useful dsRNA duplex lengths are about 19 to about 25 nucleotides. Most preferred are duplex structures with a length of 19 nucleotides. In the inventive dsRNA molecules, the antisense strand is at least partially complementary to the sense strand.

The dsRNA of the invention can contain one or more mismatches to the target sequence. In a preferred embodiment, the dsRNA of the invention contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to the terminal regions, preferably within 6, 5, 4, 3 or 2 nucleotides of the 5′ and/or 3′ terminus. For example, for a 23 nucleotide dsRNA strand which is complementary to a region of a Factor VII gene, the dsRNA preferably does not contain any mismatch within the central 13 nucleotides.

As mentioned above, at least one end/strand of the dsRNA may have a single-stranded nucleotide overhang of 1 to 5, preferably 1 or 2 nucleotides. dsRNAs having at least one nucleotide overhang have unexpectedly superior inhibitory properties than their blunt-ended counterparts. Moreover, the present inventors have discovered that the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Preferably, the single-stranded overhang is located at the 3′-terminal end of the antisense strand or, alternatively, at the 3′-terminal end of the sense strand. The dsRNA may also have a blunt end, preferably located at the 5′-end of the antisense strand. Preferably, the antisense strand of the dsRNA has a nucleotide overhang at the 3′-end, and the 5′-end is blunt. In another embodiment, one or more of the nucleotides in the overhang is replaced with a nucleoside thiophosphate.

The dsRNA of the present invention may also be chemically modified to enhance stability. The nucleic acids of the invention may be synthesized and/or modified by methods well established in the art, such as those described in “Current protocols in nucleic acid chemistry”, Beaucage, S. L. et al. (Edrs.), John Wiley & Sons, Inc., New York, N.Y., USA, which is hereby incorporated herein by reference. Chemical modifications may include, but are not limited to 2′ modifications, introduction of non-natural bases, covalent attachment to a ligand, and replacement of phosphate linkages with thiophosphate linkages. In this embodiment, the integrity of the duplex structure is strengthened by at least one, and preferably two, chemical linkages. Chemical linking may be achieved by any of a variety of well-known techniques, for example by introducing covalent, ionic or hydrogen bonds; hydrophobic interactions, van der Waals or stacking interactions; by means of metal-ion coordination, or through use of purine analogues. Preferably, the chemical groups that can be used to modify the dsRNA include, without limitation, methylene blue; bifunctional groups, preferably bis-(2-chloroethyl)amine; N-acetyl-N′-(p-glyoxylbenzoyl)cystamine; 4-thiouracil; and psoralen. In one preferred embodiment, the linker is a hexa-ethylene glycol linker. In this case, the dsRNA are produced by solid phase synthesis and the hexa-ethylene glycol linker is incorporated according to standard methods (e.g., Williams, D. J., and K. B. Hall, Biochem. (1996) 35:14665-14670). In a particular embodiment, the 5′-end of the antisense strand and the 3′-end of the sense strand are chemically linked via a hexaethylene glycol linker. In another embodiment, at least one nucleotide of the dsRNA comprises a phosphorothioate or phosphorodithioate groups. The chemical bond at the ends of the dsRNA is preferably formed by triple-helix bonds.

In certain embodiments, a chemical bond may be formed by means of one or several bonding groups, wherein such bonding groups are preferably poly-(oxyphosphinicooxy-1,3-propandiol)- and/or polyethylene glycol chains. In other embodiments, a chemical bond may also be formed by means of purine analogs introduced into the double-stranded structure instead of purines. In further embodiments, a chemical bond may be formed by azabenzene units introduced into the double-stranded structure. In still further embodiments, a chemical bond may be formed by branched nucleotide analogs instead of nucleotides introduced into the double-stranded structure. In certain embodiments, a chemical bond may be induced by ultraviolet light.

In yet another embodiment, the nucleotides at one or both of the two single strands may be modified to prevent or inhibit the activation of cellular enzymes, for example certain nucleases. Techniques for inhibiting the activation of cellular enzymes are known in the art including, but not limited to, 2′-amino modifications, 2′-amino sugar modifications, 2′-F sugar modifications, 2′-F modifications, 2′-alkyl sugar modifications, uncharged backbone modifications, morpholino modifications, 2′-O-methyl modifications, and phosphoramidate (see, e.g., Wagner, Nat. Med. (1995) 1:1116-8). Thus, at least one 2′-hydroxyl group of the nucleotides on a dsRNA is replaced by a chemical group, preferably by a 2′-amino or a 2′-methyl group. Also, at least one nucleotide may be modified to form a locked nucleotide. Such locked nucleotide contains a methylene bridge that connects the 2′-oxygen of ribose with the 4′-carbon of ribose. Introduction of a locked nucleotide into an oligonucleotide improves the affinity for complementary sequences and increases the melting temperature by several degrees.

Modifications of dsRNA molecules provided herein may positively influence their stability in vivo as well as in vitro and also improve their delivery to the (diseased) target side. Furthermore, such structural and chemical modifications may positively influence physiological reactions towards the dsRNA molecules upon administration, e.g. the cytokine release which is preferably suppressed. Such chemical and structural modifications are known in the art and are, inter alia, illustrated in Nawrot (2006) Current Topics in Med Chem, 6, 913-925.

Conjugating a ligand to a dsRNA can enhance its cellular absorption as well as targeting to a particular tissue. In certain instances, a hydrophobic ligand is conjugated to the dsRNA to facilitate direct permeation of the cellular membrane. Alternatively, the ligand conjugated to the dsRNA is a substrate for receptor-mediated endocytosis. These approaches have been used to facilitate cell permeation of antisense oligonucleotides. For example, cholesterol has been conjugated to various antisense oligonucleotides resulting in compounds that are substantially more active compared to their non-conjugated analogs. See M. Manoharan Antisense & Nucleic Acid Drug Development 2002, 12, 103. Other lipophilic compounds that have been conjugated to oligonucleotides include 1-pyrene butyric acid, 1,3-bis-O-(hexadecyl)glycerol, and menthol. One example of a ligand for receptor-mediated endocytosis is folic acid. Folic acid enters the cell by folate-receptor-mediated endocytosis. dsRNA compounds bearing folic acid would be efficiently transported into the cell via the folate-receptor-mediated endocytosis. Attachment of folic acid to the 3′-terminus of an oligonucleotide results in increased cellular uptake of the oligonucleotide (Li, S.; Deshmukh, H. M.; Huang, L. Pharm. Res. 1998, 15, 1540). Other ligands that have been conjugated to oligonucleotides include polyethylene glycols, carbohydrate clusters, cross-linking agents, porphyrin conjugates, and delivery peptides.

In certain instances, conjugation of a cationic ligand to oligonucleotides often results in improved resistance to nucleases. Representative examples of cationic ligands are propylammonium and dimethylpropylammonium. Interestingly, antisense oligonucleotides were reported to retain their high binding affinity to mRNA when the cationic ligand was dispersed throughout the oligonucleotide. See M. Manoharan Antisense & Nucleic Acid Drug Development 2002, 12, 103 and references therein.

The ligand-conjugated dsRNA of the invention may be synthesized by the use of a dsRNA that bears a pendant reactive functionality, such as that derived from the attachment of a linking molecule onto the dsRNA. This reactive oligonucleotide may be reacted directly with commercially-available ligands, ligands that are synthesized bearing any of a variety of protecting groups, or ligands that have a linking moiety attached thereto. The methods of the invention facilitate the synthesis of ligand-conjugated dsRNA by the use of, in some preferred embodiments, nucleoside monomers that have been appropriately conjugated with ligands and that may further be attached to a solid-support material. Such ligand-nucleoside conjugates, optionally attached to a solid-support material, are prepared according to some preferred embodiments of the methods of the invention via reaction of a selected serum-binding ligand with a linking moiety located on the 5′ position of a nucleoside or oligonucleotide. In certain instances, an dsRNA bearing an aralkyl ligand attached to the 3′-terminus of the dsRNA is prepared by first covalently attaching a monomer building block to a controlled-pore-glass support via a long-chain aminoalkyl group. Then, nucleotides are bonded via standard solid-phase synthesis techniques to the monomer building-block bound to the solid support. The monomer building block may be a nucleoside or other organic compound that is compatible with solid-phase synthesis.

The dsRNA used in the conjugates of the invention may be conveniently and routinely made through the well-known technique of solid-phase synthesis. It is also known to use similar techniques to prepare other oligonucleotides, such as the phosphorothioates and alkylated derivatives.

Teachings regarding the synthesis of particular modified oligonucleotides may be found in the following U.S. patents: U.S. Pat. No. 5,218,105, drawn to polyamine conjugated oligonucleotides; U.S. Pat. No. 5,541,307, drawn to oligonucleotides having modified backbones; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having β-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups may be used as linkers to other moieties attached at any of a variety of positions of the nucleoside; U.S. Pat. No. 5,587,361 drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,608,046, both drawn to conjugated 4′-desmethyl nucleoside analogs; U.S. Pat. No. 5,610,289, drawn to backbone-modified oligonucleotide analogs; U.S. Pat. No. 6,262,241 drawn to, inter alia, methods of synthesizing 2′-fluoro-oligonucleotides.

In the ligand-conjugated dsRNA and ligand-molecule bearing sequence-specific linked nucleosides of the invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. Oligonucleotide conjugates bearing a variety of molecules such as steroids, vitamins, lipids and reporter molecules, has previously been described (see Manoharan et al., PCT Application WO 93/07883). In a preferred embodiment, the oligonucleotides or linked nucleosides of the invention are synthesized by an automated synthesizer using phosphoramidites derived from ligand-nucleoside conjugates in addition to commercially available phosphoramidites.

The incorporation of a 2′-O-methyl, 2′-O-ethyl, 2′-O-allyl, 2′-O-aminoalkyl or 2′-deoxy-2′-fluoro group in nucleosides of an oligonucleotide confers enhanced hybridization properties to the oligonucleotide. Further, oligonucleotides containing phosphorothioate backbones have enhanced nuclease stability. Thus, functionalized, linked nucleosides of the invention can be augmented to include either or both a phosphorothioate backbone or a 2′-O-methyl, 2′-O-ethyl, 2′-O-aminoalkyl, 2′-O-allyl or 2′-deoxy-2′-fluoro group.

In some preferred embodiments, functionalized nucleoside sequences of the invention possessing an amino group at the 5′-terminus are prepared using a DNA synthesizer, and then reacted with an active ester derivative of a selected ligand. Active ester derivatives are well known to those skilled in the art. Representative active esters include N-hydrosuccinimide esters, tetrafluorophenolic esters, pentafluorophenolic esters and pentachlorophenolic esters. The reaction of the amino group and the active ester produces an oligonucleotide in which the selected ligand is attached to the 5′-position through a linking group. The amino group at the 5′-terminus can be prepared utilizing a 5′-Amino-Modifier C6 reagent. In a preferred embodiment, ligand molecules may be conjugated to oligonucleotides at the 5′-position by the use of a ligand-nucleoside phosphoramidite wherein the ligand is linked to the 5′-hydroxy group directly or indirectly via a linker. Such ligand-nucleoside phosphoramidites are typically used at the end of an automated synthesis procedure to provide a ligand-conjugated oligonucleotide bearing the ligand at the 5′-terminus.

In one preferred embodiment of the methods of the invention, the preparation of ligand conjugated oligonucleotides commences with the selection of appropriate precursor molecules upon which to construct the ligand molecule. Typically, the precursor is an appropriately-protected derivative of the commonly-used nucleosides. For example, the synthetic precursors for the synthesis of the ligand-conjugated oligonucleotides of the invention include, but are not limited to, 2′-aminoalkoxy-5′-ODMT-nucleosides, 2′-6-aminoalkylamino-5′-ODMT-nucleosides, 5′-6-aminoalkoxy-2′-deoxy-nucleosides, 5′-6-aminoalkoxy-2-protected-nucleosides, 3′-6-aminoalkoxy-5′-ODMT-nucleosides, and 3′-aminoalkylamino-5′-ODMT-nucleosides that may be protected in the nucleobase portion of the molecule. Methods for the synthesis of such amino-linked protected nucleoside precursors are known to those of ordinary skill in the art.

In many cases, protecting groups are used during the preparation of the compounds of the invention. As used herein, the term “protected” means that the indicated moiety has a protecting group appended thereon. In some preferred embodiments of the invention, compounds contain one or more protecting groups. A wide variety of protecting groups can be employed in the methods of the invention. In general, protecting groups render chemical functionalities inert to specific reaction conditions, and can be appended to and removed from such functionalities in a molecule without substantially damaging the remainder of the molecule.

Representative hydroxyl protecting groups, as well as other representative protecting groups, are disclosed in Greene and Wuts, Protective Groups in Organic Synthesis, Chapter 2, 2d ed., John Wiley & Sons, New York, 1991, and Oligonucleotides And Analogues A Practical Approach, Ekstein, F. Ed., IRL Press, N.Y., 1991.

Amino-protecting groups stable to acid treatment are selectively removed with base treatment, and are used to make reactive amino groups selectively available for substitution. Examples of such groups are the Fmoc (E. Atherton and R. C. Sheppard in The Peptides, S. Udenfriend, J. Meienhofer, Eds., Academic Press, Orlando, 1987, volume 9, p. 1) and various substituted sulfonylethyl carbamates exemplified by the Nsc group (Samukov et al., Tetrahedron Lett., 1994, 35:7821.

Additional amino-protecting groups include, but are not limited to, carbamate protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl. Equivalents of these amino-protecting groups are also encompassed by the compounds and methods of the invention.

Many solid supports are commercially available and one of ordinary skill in the art can readily select a solid support to be used in the solid-phase synthesis steps. In certain embodiments, a universal support is used. A universal support allows for preparation of oligonucleotides having unusual or modified nucleotides located at the 3′-terminus of the oligonucleotide. For further details about universal supports see Scott et al., Innovations and Perspectives in solid-phase Synthesis, 3rd International Symposium, 1994, Ed. Roger Epton, Mayflower Worldwide, 115-124]. In addition, it has been reported that the oligonucleotide can be cleaved from the universal support under milder reaction conditions when oligonucleotide is bonded to the solid support via a syn-1,2-acetoxyphosphate group which more readily undergoes basic hydrolysis. See Guzaev, A. I.; Manoharan, M. J. Am. Chem. Soc. 2003, 125, 2380.

The nucleosides are linked by phosphorus-containing or non-phosphorus-containing covalent internucleoside linkages. For the purposes of identification, such conjugated nucleosides can be characterized as ligand-bearing nucleosides or ligand-nucleoside conjugates. The linked nucleosides having an aralkyl ligand conjugated to a nucleoside within their sequence will demonstrate enhanced dsRNA activity when compared to like dsRNA compounds that are not conjugated.

The aralkyl-ligand-conjugated oligonucleotides of the invention also include conjugates of oligonucleotides and linked nucleosides wherein the ligand is attached directly to the nucleoside or nucleotide without the intermediacy of a linker group. The ligand may preferably be attached, via linking groups, at a carboxyl, amino or oxo group of the ligand. Typical linking groups may be ester, amide or carbamate groups.

Specific examples of preferred modified oligonucleotides envisioned for use in the ligand-conjugated oligonucleotides of the invention include oligonucleotides containing modified backbones or non-natural internucleoside linkages. As defined here, oligonucleotides having modified backbones or internucleoside linkages include those that retain a phosphorus atom in the backbone and those that do not have a phosphorus atom in the backbone. For the purposes of the invention, modified oligonucleotides that do not have a phosphorus atom in their intersugar backbone can also be considered to be oligonucleosides.

Specific oligonucleotide chemical modifications are described below. It is not necessary for all positions in a given compound to be uniformly modified. Conversely, more than one modifications may be incorporated in a single dsRNA compound or even in a single nucleotide thereof.

Preferred modified internucleoside linkages or backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free-acid forms are also included.

Representative United States patents relating to the preparation of the above phosphorus-atom-containing linkages include, but are not limited to, U.S. Pat. Nos. 4,469,863; 5,023,243; 5,264,423; 5,321,131; 5,399,676; 5,405,939; 5,453,496; 5,455,233 and 5,466,677, each of which is herein incorporated by reference.

Preferred modified internucleoside linkages or backbones that do not include a phosphorus atom therein (i.e., oligonucleosides) have backbones that are formed by short chain alkyl or cycloalkyl intersugar linkages, mixed heteroatom and alkyl or cycloalkyl intersugar linkages, or one or more short chain heteroatomic or heterocyclic intersugar linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents relating to the preparation of the above oligonucleosides include, but are not limited to, U.S. Pat. Nos. 5,034,506; 5,214,134; 5,216,141; 5,264,562; 5,466,677; 5,470,967; 5,489,677; 5,602,240 and 5,663,312, each of which is herein incorporated by reference.

In other preferred oligonucleotide mimetics, both the sugar and the internucleoside linkage, i.e., the backbone, of the nucleoside units are replaced with novel groups. The nucleobase units are maintained for hybridization with an appropriate nucleic acid target compound. One such oligonucleotide, an oligonucleotide mimetic, that has been shown to have excellent hybridization properties, is referred to as a peptide nucleic acid (PNA). In PNA compounds, the sugar-backbone of an oligonucleotide is replaced with an amide-containing backbone, in particular an aminoethylglycine backbone. The nucleobases are retained and are bound directly or indirectly to atoms of the amide portion of the backbone. Teaching of PNA compounds can be found for example in U.S. Pat. No. 5,539,082.

Some preferred embodiments of the invention employ oligonucleotides with phosphorothioate linkages and oligonucleotides with heteroatom backbones, and in particular —CH₂—NH—O—CH₂—CH₂—N(CH₃)—O—CH₂—[known as a methylene (methylimino) or MMI backbone], —CH₂—O—N(CH₃)—CH₂—CH₂—N(CH₃)—N(CH₃)—CH₂—, and —O—N(CH₃)—CH₂—CH₂—[wherein the native phosphodiester backbone is represented as—O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

The oligonucleotides employed in the ligand-conjugated oligonucleotides of the invention may additionally or alternatively comprise nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine.

Further nucleobases include those disclosed in U.S. Pat. No. 3,687,808, those disclosed in the Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligonucleotides of the invention. These include 5-substituted pyrimidines, 6-azapyrimidines and N-2, N-6 and O-6 substituted purines, including 2-aminopropyladenine, 5-propynyluracil and 5-propynylcytosine. 5-Methylcytosine substitutions have been shown to increase nucleic acid duplex stability by 0.6-1.2° C. (Id., pages 276-278) and are presently preferred base substitutions, even more particularly when combined with 2′-methoxyethyl sugar modifications.

Representative United States patents relating to the preparation of certain of the above-noted modified nucleobases as well as other modified nucleobases include, but are not limited to, the above noted U.S. Pat. No. 3,687,808, as well as U.S. Pat. Nos. 5,134,066; 5,459,255; 5,552,540; 5,594,121 and 5,596,091 all of which are hereby incorporated by reference.

In certain embodiments, the oligonucleotides employed in the ligand-conjugated oligonucleotides of the invention may additionally or alternatively comprise one or more substituted sugar moieties. Preferred oligonucleotides comprise one of the following at the 2′ position: OH; F; O-, S-, or N-alkyl, O-, S-, or N-alkenyl, or O, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. a preferred modification includes 2′-methoxyethoxy [2′-O—CH₂CH₂OCH₃, also known as 2′-O-(2-methoxyethyl) or 2′-MOE], i.e., an alkoxyalkoxy group. A further preferred modification includes 2′-dimethylaminooxyethoxy, i.e., a O(CH₂)₂ON(CH₃)₂ group, also known as 2′-DMAOE, as described in U.S. Pat. No. 6,127,533, filed on Jan. 30, 1998, the contents of which are incorporated by reference.

Other preferred modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides.

As used herein, the term “sugar substituent group” or “2′-substituent group” includes groups attached to the 2′-position of the ribofuranosyl moiety with or without an oxygen atom. Sugar substituent groups include, but are not limited to, fluoro, O-alkyl, O-alkylamino, O-alkylalkoxy, protected O-alkylamino, O-alkylaminoalkyl, O-alkyl imidazole and polyethers of the formula (O-alkyl)ₘ, wherein m is 1 to about 10. Preferred among these polyethers are linear and cyclic polyethylene glycols (PEGs), and (PEG)-containing groups, such as crown ethers and, inter alia, those which are disclosed by Delgardo et. al. (Critical Reviews in Therapeutic Drug Carrier Systems 1992, 9:249), which is hereby incorporated by reference in its entirety. Further sugar modifications are disclosed by Cook (Anti-fibrosis Drug Design, 1991, 6:585-607). Fluoro, O-alkyl, O-alkylamino, O-alkyl imidazole, O-alkylaminoalkyl, and alkyl amino substitution is described in U.S. Pat. No. 6,166,197, entitled “Oligomeric Compounds having Pyrimidine Nucleotide(s) with 2′ and 5′ Substitutions,” hereby incorporated by reference in its entirety.

Additional sugar substituent groups amenable to the invention include 2′-SR and 2′-NR₂ groups, wherein each R is, independently, hydrogen, a protecting group or substituted or unsubstituted alkyl, alkenyl, or alkynyl. 2′-SR Nucleosides are disclosed in U.S. Pat. No. 5,670,633, hereby incorporated by reference in its entirety. The incorporation of 2′-SR monomer synthons is disclosed by Hamm et al. (J. Org. Chem., 1997, 62:3415-3420). 2′-NR nucleosides are disclosed by Goettingen, M., J. Org. Chem., 1996, 61, 6273-6281; and Polushin et al., Tetrahedron Lett., 1996, 37, 3227-3230. Further representative 2′-substituent groups amenable to the invention include those having one of formula I or II:

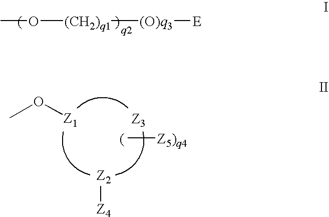

wherein,

E is C₁-C₁₀ alkyl, N(Q₃)(Q₄) or N═C (Q₃)(Q₄); each Q₃ and Q₄ is, independently, H, C₁-C₁₀ alkyl, dialkylaminoalkyl, a nitrogen protecting group, a tethered or untethered conjugate group, a linker to a solid support; or Q₃ and Q₄, together, form a nitrogen protecting group or a ring structure optionally including at least one additional heteroatom selected from N and O;

q₁ is an integer from 1 to 10;
q₂ is an integer from 1 to 10;
q₃ is 0 or 1;
q₄ is 0, 1 or 2;
each Z₁, Z₂ and Z₃ is, independently, C₄-C₇ cycloalkyl, C₅-C₁₄ aryl or C₃-C₁₅ heterocyclyl, wherein the heteroatom in said heterocyclyl group is selected from oxygen, nitrogen and sulfur; Z₄ is OM₁, SM₁, or N(M₁)₂; each M₁ is, independently, H, C₁-C₈ alkyl, C₁-C₈ haloalkyl, C(═NH)N(H)M₂, C(═O)N(H)M₂ or OC(═O)N(H)M₂; M₂ is H or C₁-C₈ alkyl; and Z₅ is C₁-C₁₀ alkyl, C₁-C₁₀ haloalkyl, C₂-C₁₀ alkenyl, C₂-C₁₀ alkynyl, C₆-C₁₄ aryl, N(Q₃)(Q₄), OQ₃, halo, SQ₃ or CN.

Representative 2′-O-sugar substituent groups of formula I are disclosed in U.S. Pat. No. 6,172,209, entitled “Capped 2′-Oxyethoxy Oligonucleotides,” hereby incorporated by reference in its entirety. Representative cyclic 2′-O-sugar substituent groups of formula II are disclosed in U.S. Pat. No. 6,271,358, entitled “RNA Targeted 2′-Modified Oligonucleotides that are Conformationally Preorganized,” hereby incorporated by reference in its entirety.

Sugars having O-substitutions on the ribosyl ring are also amenable to the invention. Representative substitutions for ring O include, but are not limited to, S, CH₂, CHF, and CF₂. Oligonucleotides may also have sugar mimetics, such as cyclobutyl moieties, in place of the pentofuranosyl sugar. Representative United States patents relating to the preparation of such modified sugars include, but are not limited to, U.S. Pat. Nos. 5,359,044; 5,466,786; 5,519,134; 5,591,722; 5,597,909; 5,646,265 and 5,700,920, all of which are hereby incorporated by reference.

Additional modifications may also be made at other positions on the oligonucleotide, particularly the 3′ position of the sugar on the 3′ terminal nucleotide. For example, one additional modification of the ligand-conjugated oligonucleotides of the invention involves chemically linking to the oligonucleotide one or more additional non-ligand moieties or conjugates which enhance the activity, cellular distribution or cellular uptake of the oligonucleotide. Such moieties include but are not limited to lipid moieties, such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-5-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Len., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

The invention also includes compositions employing oligonucleotides that are substantially chirally pure with regard to particular positions within the oligonucleotides. Examples of substantially chirally pure oligonucleotides include, but are not limited to, those having phosphorothioate linkages that are at least 75% Sp or Rp (Cook et al., U.S. Pat. No. 5,587,361) and those having substantially chirally pure (Sp or Rp) alkylphosphonate, phosphoramidate or phosphotriester linkages (Cook, U.S. Pat. Nos. 5,212,295 and 5,521,302).

In certain instances, the oligonucleotide may be modified by a non-ligand group. A number of non-ligand molecules have been conjugated to oligonucleotides in order to enhance the activity, cellular distribution or cellular uptake of the oligonucleotide, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-5-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Typical conjugation protocols involve the synthesis of oligonucleotides bearing an aminolinker at one or more positions of the sequence. The amino group is then reacted with the molecule being conjugated using appropriate coupling or activating reagents. The conjugation reaction may be performed either with the oligonucleotide still bound to the solid support or following cleavage of the oligonucleotide in solution phase. Purification of the oligonucleotide conjugate by HPLC typically affords the pure conjugate. The use of a cholesterol conjugate is particularly preferred since such a moiety can increase targeting to tissues in the liver, a site of Factor VII protein production.

Alternatively, the molecule being conjugated may be converted into a building block, such as a phosphoramidite, via an alcohol group present in the molecule or by attachment of a linker bearing an alcohol group that may be phosphorylated.

Importantly, each of these approaches may be used for the synthesis of ligand conjugated oligonucleotides. Amino linked oligonucleotides may be coupled directly with ligand via the use of coupling reagents or following activation of the ligand as an NHS or pentfluorophenolate ester. Ligand phosphoramidites may be synthesized via the attachment of an aminohexanol linker to one of the carboxyl groups followed by phosphitylation of the terminal alcohol functionality. Other linkers, such as cysteamine, may also be utilized for conjugation to a chloroacetyl linker present on a synthesized oligonucleotide.

One of the major gists of the present invention is the provision of pharmaceutical compositions which comprise the dsRNA molecules of this invention. Such a pharmaceutical composition may also comprise individual strands of such a dsRNA molecule or (a) vector(s) that comprise(s) a regulatory sequence operably linked to a nucleotide sequence that encodes at least one of a sense strand or an antisense strand comprised in the dsRNA molecules of this invention. Also cells and tissues which express or comprise the herein defined dsRNA molecules may be used as pharmaceutical compositions. Such cells or tissues may in particular be useful in the transplantation approaches. These approaches may also comprise xeno transplantations.

In one embodiment, the invention provides pharmaceutical compositions comprising a dsRNA, as described herein, and a pharmaceutically acceptable carrier. The pharmaceutical composition comprising the dsRNA is useful for treating a disease or disorder associated with the expression or activity of a FVII gene, such as thromboembolitic disorders.

The pharmaceutical compositions of the invention are administered in dosages sufficient to inhibit expression of a FVII gene. The present inventors have found that, because of their improved efficiency, compositions comprising the dsRNA of the invention can be administered at low dosages.

In general, a suitable dose of dsRNA will be in the range of 0.01 to 5.0 milligrams per kilogram body weight of the recipient per day, preferably in the range of 0.1 to 200 micrograms per kilogram body weight per day, more preferably in the range of 0.1 to 100 micrograms per kilogram body weight per day, even more preferably in the range of 1.0 to 50 micrograms per kilogram body weight per day, and most preferably in the range of 1.0 to 25 micrograms per kilogram body weight per day. The pharmaceutical composition may be administered once daily, or the dsRNA may be administered as two, three, four, five, six or more sub-doses at appropriate intervals throughout the day or even using continuous infusion. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art. In this embodiment, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The data obtained from cell culture assays and animal studies can be used in formulation a range of dosage for use in humans. The dosage of compositions of the invention lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, the dsRNAs of the invention can be administered in combination with other known agents. In any event, the administering physician can adjust the amount and timing of dsRNA administration on the basis of results observed using standard measures of efficacy known in the art or described herein.

The pharmaceutical compositions encompassed by the invention may be administered by any means known in the art including, but not limited to oral or parenteral routes, including intravenous, intramuscular, intraperitoneal, subcutaneous, transdermal, airway (aerosol), nasal, rectal, vaginal and topical (including buccal and sublingual) administration, and epidural administration. In preferred embodiments, the pharmaceutical compositions are administered intravenously by infusion or injection.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The above provided embodiments and items of the present invention are now illustrated with the following, non-limiting examples.

### DESCRIPTION OF FIGURES AND APPENDED TABLES

FIG. 1—Effect of dsRNA targeting FVII (“FVII dsRNA”) on FVII plasma levels in guinea pigs after i. v. injection of FVII dsRNA comprising Seq. ID pair 259/260 (FIG. 1a) and dsRNA comprising Seq. ID pair 253/254 (FIG. 1b) at 4 mg/kg in a LNP01 (1:14) liposome formulation. Luciferase dsRNA (SEQ ID pairs 411/412)/LNP01 and PBS are controls. Results are from individual animals.

FIG. 2-Effect of FVII dsRNA in guinea pigs on FVII mRNA levels in liver (2a) and FVII levels in plasma (2b) after i. v. injection of FVII dsRNA comprising Seq. ID pair 259/260 (“FVII siRNA”) at 1, 2, 3, 4, 5 mg/kg in a LNP01 (1:14) liposome formulation. All measurements were performed 48 hrs or 72 hours post-injection. mRNA results are expressed in percent of the PBS-treated group; FVII zymogen results are expressed in percent of the pre-treatment value. Luciferase dsRNA (SEQ ID pairs 411/412; “Luc siRNA”)/LNP01 and PBS are controls. Statistic: mean±sem; *ANOVA, post-hoc Dunnett's test; ‡ Multiple t-test.

FIG. 3—Effect of FVII dsRNA on prothrombin time (PT) of guinea pigs after i. v. injection of FVII dsRNA comprising Seq. ID pair 259/260 (“FVII siRNA”) at 1, 2, 3, 4, 5 mg/kg in a LNP01 (1:14) liposome formulation. Blood was collected immediately before i. v. injection of FVII dsRNA (baseline) and 48 hrs or 72 hours post-injection. Results are expressed in fold prolongation of pre-treatment values (mean±sem). Luciferase dsRNA (SEQ ID pairs 411/412; “Luc siRNA”)/LNP01 and PBS are controls.

FIG. 4—Antithrombotic effects of FVII dsRNA in the guinea pig arterial thrombosis model after i. v. injection of FVII dsRNA comprising Seq. ID pair 259/260 (“FVII dsRNA”) at 1, 2, 3, 4, 5 mg/kg in a LNP01 (1:14) liposome formulation. All measurements were performed in anesthetized animals 48 hrs or 72 hours post-injection (see methods). Results are expressed in percent of the PBS-treated group. Luciferase dsRNA (SEQ ID pairs 411/412; “Luc dsRNA”)/LNP01 and PBS are controls. Statistic: mean±sem; *ANOVA, post-hoc Dunnett's test; ‡ Multiple t-test.

FIG. 5—Effect of FVII dsRNA in guinea pigs on FVII mRNA levels in liver (a) and FVII levels in plasma (b) after is v. injection of FVII dsRNA comprising Seq. ID pair 259/260 (“siFVII”) at 1, 2, 3, 4, 5 mg/kg in a SNALP-L formulation. Luciferase dsRNA (SEQ ID pairs 411/412; “siLuc”)/SNALP-L and PBS are controls.

FIG. 6—Effect of FVII dsRNA on (a) surgical blood loss and (b) nail cuticle bleeding time in guinea pigs after i.v. injection of FVII dsRNA comprising Seq. ID pair 259/260 in a SNALP-L formulation. Results were expressed in fold-increase (surgical blood loss) and fold-prolongation (cuticle bleeding time) of the PBS-treated group. All measurements were performed 72 hours post-injection. Luciferase dsRNA (Seq. ID pairs 411/412) in a SNALP-L formulation (Luc dsRNA) and PBS are controls. With up to 95% FVII down regulation (0.05 mg/kg to 2 mg/kg FVII dsRNA), no increase in bleeding-propensity was observed in both models.

FIG. 7—Correlation between FVII activity in plasma and PT-prolongation. FVII activity decrease after iv injection of FVII dsRNA (combined data from FVII dsRNA formulated in LNP01 and SNALP-L) correlated well with FVII-dependent coagulation parameter PT.

FIG. 8—FVII activity in cynomolgus monkey plasma measured by chromogenic assay 3 times pre dosing and at 24 hours and 48 hours post single iv bolus injection of Luciferase dsRNA (Seq. ID pair 411/412) or FVII dsRNA (Seq. IDs 19/20). Dose with respect to dsRNA given for each group as mg/kg. N=2 female cynomolgus monkeys. Values are normalized to mean of predose FVII activity values of each individual monkey, with error bars indicating standard deviation.

FIG. 9—Prothrombin time (PT) in cynomolgus plasma measured 3 times pre dosing and at 24 hours and 48 hours post single iv bolus injection of Luciferase dsRNA in a SNALP formulation (siLUC) (Seq. ID pair 411/412) or FVII dsRNA in a SNALP formulation (siFVII) (Seq. IDs 19/20). Dose with respect to dsRNA is given for each group as mg/kg. N=2 female cynomolgus monkeys. Values are given as fold change normalized to mean of predose PT of each individual monkey, with error bars indicating standard deviation.

FIG. 10—FVII activity in cynomolgus monkey plasma measured by chromogenic assay 3 times before dosing and at 24 hours and 48 hours after a single iv bolus injection of Luciferase dsRNA in a SNALP formulation (siLUC) (Seq. ID pair 411/412) or FVII dsRNA in a SNALP formulation (siFVII) (Seq. IDs 19/20). Dose with respect to dsRNA was given for each group as mg/kg. N=2 male cynomolgus monkeys, except for the 1 mg/kg FVII dsRNA group where n=3 male cynomolgus monkeys and the 3 mg/kg Luciferase dsRNA group where n=2 female cynomolgus monkeys. Values were normalized to the mean of predose FVII activity values of each individual monkey set to 100%. Error bars indicate min/max values of monkeys in each group.

FIG. 11—Prothrombin time (PT) in cynomolgus monkey plasma measured 3 times before dosing and at 24 hours and 48 hours after a single iv bolus injection of for Luciferase dsRNA in a SNALP formulation (siLUC) (Seq. ID pair 411/412) or FVII dsRNA in a SNALP formulation (siFVII) (Seq. IDs 19/20). Dose with respect to dsRNA is given for each group as mg/kg. N=2 male cynomolgus monkeys, except for the 1 mg/kg FVII dsRNA group where n=3 male cynomolgus monkeys and the 3 mg/kg Luciferase dsRNA group where n=2 female cynomolgus monkeys. Values are given as x-fold PT change normalized to mean of predose PT values of each individual monkey set to 1. Error bars indicate min/max values of monkeys in each group.

FIG. 12—FVII activity in cynomolgus serum was followed over time before and after a single iv bolus injection of Luciferase dsRNA in a SNALP formulation (siLUC) (Seq. ID pair 411/412) or FVII dsRNA in a SNALP formulation (siFVII) (Seq. IDs 19/20). FVII activity was measured by chromogenic assay 3 times before dosing and at indicated time points after dosing. Dose with respect to dsRNA is given for each animal as mg/kg and numbers indicate individual animal-ID in study. Curves are normalized to mean of predose of each animal set to 100% at day of injection.

FIG. 13—Prothrombin time (PT) in cynomolgus plasma was followed over time before and after a single iv bolus injection of Luciferase dsRNA in a SNALP formulation (siLUC) (Seq. ID pair 411/412) or FVII dsRNA in a SNALP formulation (siFVII) (Seq. IDs 19/20). PT was measured 3 times before dosing and at indicated time points after dosing. Dose with respect to dsRNA is given for each animal as mg/kg and numbers indicate individual animal-ID in study. Values are given as fold PT change and curves are normalized to mean of predose of each animal set to 1 at day of injection.

FIG. 14—FVII activity in cynomolgus monkey plasma was followed over time before and after repeated iv bolus injections of FVII dsRNA in a SNALP formulation (siFVII) (Seq. IDs 19/20) at 3 mg/kg. FVII activity was measured by chromogenic assay 3 times pre dosing and at indicated time points post dosing. Curves are normalized to mean of predose of each animal set to 100% at day of first injection.

FIG. 15—Prothrombin time (PT) in cynomolgus monkey plasma was followed over time before and after repeated iv bolus injections of FVII dsRNA in a SNALP formulation (siFVII) (Seq. IDs 19/20). PT was measured 3 times before dosing and at indicated time points after dosing with 3 mg/kg. Values are given as fold PT change and curves are normalized to mean of predose of each animal set to 1 at day of injection.

FIG. 16—Effect of FVII dsRNA comprising SEQ ID pair 13/14 on silencing off-target sequences. Expression of renilla luciferase protein after transfection of COS7 cells expressing dual-luciferase constructs, representative for either 19 mer target site of FVII mRNA (“on”) or in silico predicted off-target sequences (“off 1” to “off 10”; with “off 1”-“off 8” being antisense strand off-targets and “off 9” to “off 10” being sense strand off-targets), with 50 nM FVII dsRNA. Perfect matching off-target dsRNAs are positive controls for functional silencing of the corresponding target-site.

FIG. 17—Effect of FVII dsRNA comprising SEQ ID pair 19/20 on silencing off-target sequences. Expression of renilla luciferase protein after transfection of COS7 cells expressing dual-luciferase constructs, representative for either 19 mer target site of FVII mRNA (“on”) or in silico predicted off-target sequences (“off 1” to “off 17”; with “off 1”-“off 14” being antisense strand off-targets and “off 15” to “off 17” being sense strand off-targets), with 50 nM FVII dsRNA. Perfect matching off-target dsRNAs are positive controls for functional silencing of the corresponding target-site. Target site of Factor VII mRNA was cloned with the same 10 nucleotides upstream and downstream as off 11 to generate a functional target site.

FIG. 18—Effect of FVII dsRNA comprising SEQ ID pair 11/12 on silencing off-target sequences. Expression of renilla luciferase protein after transfection of COST cells expressing dual-luciferase constructs, representative for either 19 mer target site of FVII mRNA (“on”) or in silico predicted off-target sequences (“off 1” to “off 16”; with “off 1” “off 13” being antisense strand off-targets and “off 14” to “off 16” being sense strand off-targets), with 50 nM FVII dsRNA. Perfect matching off-target dsRNAs are positive controls for functional silencing of the corresponding target-site. Target site of Factor VII mRNA was cloned with the same 10 nucleotides upstream and downstream as off 11 for SEQ ID pair 19/20 to generate a functional target site.

Table 1—dsRNA targeting human Factor VII gene. Letters in capitals represent RNA nucleotides, lower case letters “c”, “g”, “a” and “u” represent 2′ O-methyl-modified nucleotides, “s” represents phosphorothioate and “dT” deoxythymidine.

Table 2—Characterization of dsRNAs targeting human Factor VII: Activity testing for dose response in Huh7 cells. IC 50: 50% inhibitory concentration.

Table 3—Characterization of dsRNAs targeting human Factor VII: Stability and Cytokine Induction. t 1/2: half-life of a strand as defined in examples, PBMC: Human peripheral blood mononuclear cells.

Table 4—dsRNAs targeting guinea pig Factor VII gene. Letters in capitals represent RNA nucleotides, lower case letters “c”, “g”, “a” and “u” represent 2′ O-methyl-modified nucleotides, “s” represents phosphorothioate and “dT” deoxythymidine. “f” represents 2′ fluoro modification of the preceding nucleotide.

Table 5—Characterization of dsRNA targeting guinea pig Factor VII. IC 50: 50% inhibitory concentration, PBMC: Human peripheral blood mononuclear cells.

Table 6 —dsRNA targeting human Factor VII gene. Letters in capitals represent RNA nucleotides and “T” represents deoxythymidine.

Table 7—dsRNAs targeting guinea pig Factor VII gene. Letters in capitals represent RNA nucleotides “T” represents deoxythymidine.

Table 8—Selected off-targets of dsRNAs targeting human FVII comprising sequence ID pair 13/14

Table 9—Selected off-targets of dsRNAs targeting human FVII comprising sequence ID pair 19/20.

Table 10—Selected off-targets of dsRNAs targeting human FVII comprising sequence ID pair 11/12.

### EXAMPLES

### Identification of dsRNAs for Therapeutic Use

dsRNA design was carried out to identify dsRNAs specifically targeting human Factor VII for therapeutic use. First, the known mRNA sequences of human (Homo sapiens) Factor VII (NM_{—}019616 and NM_{—}000131.3 listed as SEQ ID NO. 406 and SEQ ID NO. 407) were examined by computer analysis to identify homologous sequences of 19 nucleotides that yield RNA interference (RNAi) agents cross-reactive between these sequences.

In identifying RNAi agents, the selection was limited to 19mer sequences having at least 2 mismatches to any other sequence in the human RefSeq database (release 25), which we assumed to represent the comprehensive human transcriptome, by using the fastA algorithm.

CDS (coding sequence) of cynomolgous monkey (Macaca fascicularis) Factor VII gene was sequenced after RT-PCR amplification from 16 monkeys. This sequence together with reverse complement of NCBI EST/EMBL BB885059 EST (SEQ ID NO. 408) was used to generated a representative consensus sequence (see Seq. ID 409) for cynomolgous monkey Factor VII.

dsRNAs cross-reactive to human as well as cynomolgous monkey Factor VII were defined as most preferable for therapeutic use. All sequences containing 4 or more consecutive G's (poly-G sequences) were excluded from the synthesis.

The sequences thus identified formed the basis for the synthesis of the RNAi agents in Tables 1 and 6.

Identification of dsRNAs for In Vivo Proof of Concept Studies

dsRNA design was carried out to identify dsRNAs targeting guinea pig (Cavia porcellus) for in vivo proof-of-concept experiments as well as human Factor VII for preceding in vitro screening purposes. First, the predicted transcript for guinea pig Factor VII ENSEMBL (ENSCPOT00000005353, SEQ ID NO. 410) and both known mRNA sequences of human Factor VII (NM_{—}019616 and NM_{—}000131.3 listed as SEQ ID NO. 406 and SEQ ID NO. 407) were examined by computer analysis to identify homologous sequences of 19 nucleotides that yield RNAi agents cross-reactive between these sequences.

All sequences containing 4 or more consecutive G's (poly-G sequences) were excluded from the synthesis. The sequences thus identified formed the basis for the synthesis of the RNAi agents in Tables 4 and 7.

dsRNA Synthesis

Where the source of a reagent is not specifically given herein, such reagent may be obtained from any supplier of reagents for molecular biology at a quality/purity standard for application in molecular biology.

Single-stranded RNAs were produced by solid phase synthesis on a scale of 1 mole using an Expedite 8909 synthesizer (Applied Biosystems, Applera Deutschland GmbH, Darmstadt, Germany) and controlled pore glass (CPG, 500 Å, Proligo Biochemie GmbH, Hamburg, Germany) as solid support. RNA and RNA containing 2′-O-methyl nucleotides were generated by solid phase synthesis employing the corresponding phosphoramidites and 2′-O-methyl phosphoramidites, respectively (Proligo Biochemie GmbH, Hamburg, Germany). These building blocks were incorporated at selected sites within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, N.Y., USA. Phosphorothioate linkages were introduced by replacement of the iodine oxidizer solution with a solution of the Beaucage reagent (Chruachem Ltd, Glasgow, UK) in acetonitrile (1%). Further ancillary reagents were obtained from Mallinckrodt Baker (Griesheim, Germany).

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, Unterschleiβheim, Germany). Double stranded RNA was generated by mixing an equimolar solution of complementary strands in annealing buffer (20 mM sodium phosphate, pH 6.8; 100 mM sodium chloride), heated in a water bath at 85-90° C. for 3 minutes and cooled to room temperature over a period of 3-4 hours. The annealed RNA solution was stored at −20° C. until use.

### Activity Testing

The activity of the Factor VII-dsRNAs described above was tested in Huh7 cells. Huh7 cells in culture were used for quantification of Factor VII mRNA by branched DNA in total mRNA derived from cells incubated with factor VII-specific dsRNAs.

Huh7 cells were obtained from American Type Culture Collection (Rockville, Md., cat. No. HB-8065) and cultured in DMEM/F-12 without Phenol red (Gibco Invitrogen, Germany, cat. No. 11039-021) supplemented to contain 5% fetal calf serum (FCS) (Gibco Invitrogen cat. No. 16250-078), 1% Penicillin/Streptomycin (Gibco Invitrogen, cat. No. 15140-122) at 37° C. in an atmosphere with 5% CO.sub.2 in a humidified incubator (Heraeus HERAcell, Kendro Laboratory Products, Langenselbold, Germany).

Cell seeding and transfection of dsRNA were performed at the same time. For transfection with dsRNA, Huh7 cells were seeded at a density of 2.5.times.10.sup.4 cells/well in 96-well plates. Transfection of dsRNA was carried out with lipofectamine 2000 (Invitrogen GmbH, Karlsruhe, Germany, cat. No. 11668-019) as described by the manufacturer. In a first single dose experiment dsRNAs were transfected at a concentration of 30 nM in Huh7 cells. Each datapoint was determined in quadruplicate. Two independent experiments were performed. Most effective dsRNAs showing a mRNA knockdown of more than 70% from single dose screen at 30 nM were further characterized by dose response curves. For dose response curves, transfections were performed as described for the single dose screen above, but with the following concentrations of dsRNA (nM): 24, 6, 1.5, 0.375, 0.0938, 0.0234, 0.0059, 0.0015, 0.0004 and 0.0001 nM. After transfection cells were incubated for 24 h at 37° C. and 5% CO2 in a humidified incubator (Heraeus GmbH, Hanau, Germany). For measurement of Factor VII mRNA the more sensitive QuantiGene 2.0 Assay Kit (Panomics, Fremont, Calif., USA, cat. No. QS0011) for bDNA quantitation of mRNA was used whereas for measurement of GAP-DH mRNA QuantiGene 1.0 Assay Kit was used (Panomics, Fremont, Calif., USA, Cat-No: QG0004). Transfected Huh7 cells were harvested and lysed at 53° C. following procedures recommended by the manufacturer. 50 μl of the lysates were incubated with probesets specific to human Factor VII mRNA, or guinea pig Factor VII respectively (sequence of probesets see below) and processed according to the manufacturer's protocol for QuantiGene. For measurement of GAP-DH mRNA 10 μl of the cell lysate was analyzed with the GAP-DH specific probeset. Chemo luminescence was measured in a Victor2-Light (Perkin Elmer, Wiesbaden, Germany) as RLUs (relative light units) and values obtained with the human factor VII probeset were normalized to the respective human GAPDH values for each well. Unrelated control dsRNAs were used as a negative control. Inhibition data are given in tables 2 and 5.

Sequences of bDNA probes for determination of human Factor
VII

| FPL | | | SEQ ID |
|---|---|---|---|
| Name | Function | Sequence | No. |
| F71 | LE | TCGGGCAGGCAGAGGGTTTTTGAAGTTACCGTTTT | 349 |
| F72 | LE | CGTCCTCTCAGAGAACGTCCGTTTTTTCTCAGTCAAAGCAT | 350 |
| F73 | CE | AAGCGCACGAAGGCCAGTTTTTCTCTTGGAAAGAAAGT | 351 |
| F74 | CE | CCAGCCGCTGACCAATGAGTTTTTCTCTTGGAAAGAAAGT | 352 |
| F75 | LE | CGGTCCAGCAGCTGGCCTTTTTGAAGTTACCGTTTT | 353 |
| F76 | LE | GGGCCGTGGCGCCATTTTTCTGAGTCAAAGCAT | 354 |
| F77 | CE | CGTTGAGGACCATGAGCTCCATTTTTCTCTTGGAAAGAAAGT | 355 |
| F78 | BL | GGTCATCAGCCGGGGCA | 356 |
| F79 | BL | GACTGCTGCAGGCAGTCCTG | 357 |
| F710 | LE | GGGAGTCTCCCACCTTCCGTTTTTTGAAGTTACCGTTTT | 358 |
| F711 | LE | CAGAACATGTACTCCGTGATATTTGTTTTTCTGAGTCAAAGCAT | 359 |
| F712 | CE | CCATCCGAGTAGCCGGCATTTTTCTCTTGGAAAGAAAGT | 360 |
| F713 | LE | CCTTCCAGGAGTCCTTGCTGTTTTTGAAGTTACCGTTTT | 361 |
| F714 | LE | GTGGGCCTCCACTGTCCCTTTTTCTGAGTCAAAGCAT | 362 |
| F715 | CE | CCCGGTAGTGGGTGGCATTTTTTCTCTTGGAAAGAAAGT | 363 |
| F716 | LE | CCCGTCAGGTACCACGTGCTTTTTGAAGTTACCGTTTT | 364 |
| F717 | LE | TGGCCCCAGCTGACGATGTTTTTCTGAGTCAAAGCAT | 365 |
| F718 | CE | CACGGTTGCGCAGCCCTTTTTCTCTTGGAAAGAAAGT | 366 |
| F719 | LE | GTGTACACCCCAAAGTGGCCTTTTTGAAGTTACCGTTTT | 367 |
| F720 | LE | TCGATGTACTGGGAGACCCTGTTTTTCTGAGTCAAAGCAT | 368 |

Sequences of bDNA probes for determination of human GAPDH

| | | | SEQ |
|---|---|---|---|
| | | | ID |
| FPL Name | Function | Sequence | No. |
| hGAP001 | CE | GAATTTGCCATGGGTGGAATTTTTTCTCTTGGAAAGAAAGT | 369 |
| hGAP002 | CE | GGAGGGATCTCGCTCCTGGATTTTTCTCTTGGAAAGAAAGT | 370 |
| hGAP003 | CE | CCCCAGCCTTCTCCATGGTTTTTTCTCTTGGAAAGAAAGT | 371 |
| hGAP004 | CE | GCTCCCCCCTGCAAATGAGTTTTTCTCTTGGAAAGAAAGT | 372 |
| hGAP005 | LE | AGCCTTGACGGTGCCATGTTTTTAGGCATAGGACCCGTGTCT | 373 |
| hGAP006 | LE | GATGACAAGCTTCCCGTTCTCTTTTTAGGCATACGACCCGTGTCT | 374 |
| hGAP007 | LE | AGATGGTGATGGGATTTCCATTTTTTTAGGCATAGGACCCGTGTCT | 375 |
| hGAP008 | LE | GCATCGCCCCACTTGATTTTTTTTTAGGCATAGGACCCGTGTCT | 376 |
| hGAP009 | LE | CACGACGTACTCAGCGCCATTTTTAGGCATAGGACCCGTGTCT | 377 |
| hGAP010 | LE | GGCAGAGATGATGACCCTTTTGTTTTTAGGCATAGGACCCGTGTCT | 378 |
| hGAP011 | BL | GGTGAAGACGCCAGTGGACTC | 379 |

LE = label extender, CE = capture extender, BL = blocking probe

Stability of dsRNAs

Stability of dsRNAs was determined in in vitro assays with either human serum or plasma from cynomolgous monkey by measuring the half-life of each single strand.

Measurements were carried out in triplicates for each time point, using 30 μl 50 μM dsRNA sample mixed with 300 human serum or cynomolgous plasma (Sigma Aldrich). Mixtures were incubated for either 0 min, 30 min, 1 h, 3 h, 6 h, 24 h, or 48 h at 37° C. As control for unspecific degradation dsRNA was incubated with 30 μl 1×PBS pH 6.8 for 48 h. Reactions were stopped by the addition of 4 μl proteinase K (20 mg/ml), 25 μl of “Tissue and Cell Lysis Solution” (Epicentre) and 38 μl Millipore water for 30 min at 65° C. Samples were afterwards spin filtered through a 0.2 μm 96 well filter plate at 1400 rpm for 8 min, washed with 55 μl Millipore water twice and spin filtered again.

For separation of single strands and analysis of remaining full length product (FLP), samples were run through an ion exchange Dionex Summit HPLC under denaturing conditions using as eluent A 20 mM Na3PO4 in 10% ACN pH=11 and for eluent B 1 M NaBr in eluent A.

The following gradient was applied:

| Time | % A | % B |
|---|---|---|
| −1.0 min | 75 | 25 |
| 1.00 min | 75 | 25 |
| 19.0 min | 38 | 62 |
| 19.5 min | 0 | 100 |
| 21.5 min | 0 | 100 |
| 22.0 min | 75 | 25 |
| 24.0 min | 75 | 25 |

For every injection, the chromatograms were integrated automatically by the Dionex Chromeleon 6.60 HPLC software, and were adjusted manually if necessary. All peak areas were corrected to the internal standard (IS) peak and normalized to the incubation at t=0 min. The area under the peak and resulting remaining FLP was calculated for each single strand and triplicate separately. Half-life (t1/2) of a strand was defined by the average time point [h] for triplicates at which half of the FLP was degraded. Results are given in tables 3 and 5.

### Cytokine Induction

Potential cytokine induction of dsRNAs was determined by measuring the release of INF-α and TNF-α in an in vitro PBMC assay.

Human peripheral blood mononuclear cells (PBMC) were isolated from buffy coat blood of two donors by Ficoll centrifugation at the day of transfection. Cells were transfected in quadruplicates with dsRNA and cultured for 24 h at 37° C. at a final concentration of 130 nM in Opti-MEM, using either Gene Porter 2 (GP2) or DOTAP. dsRNA sequences that were known to induce INF-α and TNF-α in this assay, as well as a CpG oligo, were used as positive controls. Chemical conjugated dsRNA or CpG oligonucleotides that did not need a transfection reagent for cytokine induction, were incubated at a concentration of 500 nM in culture medium. At the end of incubation, the quadruplicate culture supernatant were pooled.

INF-α and TNF-α was then measured in these pooled supernatants by standard sandwich ELISA with two data points per pool. The degree of cytokine induction was expressed relative to positive controls using a score from 0 to 5, with 5 indicating maximum induction. Results are given in tables 3 and 5.

In Vivo Effects of dsRNA Targeting FVII (Guinea Pig)

### Antithrombotic Effects

The activity of the FVII dsRNA described above was tested in a validated guinea pig arterial thrombosis model previously developed for the assessment of the in vivo efficacy of novel antithrombotic drugs (Himber J. et al., Thromb Haemost. (2001); 85:475-481).

Male guinea pigs (350-450 g, CRL: (HA) BR, Charles River (Germany) were anesthetized by i. m. induction with ketamine-HCl 90 mg/kg and Xylazine 2% 10 mg/kg, followed by continuous gaz anesthesia. 1-3 Vol % isoflurane in O₂/air 40:60 was delivered via a vaporizer through a double inhalation mask which supplies the anesthetic and scavenges excess vapors simultaneously (Provet AG, Switzerland). Body temperature was thermostatically kept at 38° C.

The guinea pig was placed in dorsal position and a catheter (TriCath In 22G, 0.8 mm×30 mm, Codan Steritex ApS, Espergaerde, Denmark) was placed into the right femoral artery for blood sampling. The right carotid artery was dissected free and a perivascular ultrasonic flowprobe (Transonic 0.7 PSB 232) coupled to a Transit Time flowmeter module (TS420, Transonic Systems Inc. Ithaca, N.Y.,USA) was placed around the carotid artery to monitor the blood flow velocity. The carotid blood flow velocity were recorded on a Graphtec Linear recorder VII (Model WR 3101, Hugo Sachs, March-Hugstetten, Germany).

After a 5 to 15 minutes stabilization period of the blood flow, a damage of the subendothelium was induced two millimeters distal to the flow probe by pinching a 1-mm segment of the dissected carotid artery with a rubber-covered forceps for 10 seconds. After damage a gradual decline of blood flow occurs resulting in complete vessel occlusion. When flow reached zero, a mild shaking of the carotid artery on the damaged area dislodged the occlusive thrombus and restored the flow resulting in cyclic flow variations (CFVs). When no CFVs were observed for 8 minutes, the pinching was repeated at the site of the first damage. If no CFVs occurred then the same procedure was repeated every 8 minutes. Finally, the number of pinches necessary to produce the CFVs were counted over the 40-minute observation period. Using this protocol, the average periodicity of each CFV was approximately 3 to 5 min/cycle in control animals. A thrombosis index was calculated as the ratio of the number of CFVs to the number of pinches.

The FVII dsRNA described above was injected in the jugular vein of anesthetized guinea pigs 48 or 72 hours prior to vessel wall injury. Blood was collected on a 108 mM sodium citrate solution (1:10 volume) before start of drug injection and before vessel wall injury.

### Bleeding Time and Blood Loss

The nail cuticle bleeding time (NCBT) was performed as previously described (Himber J. et al., Thromb Haemost. (1997) 78:1142-1149). NCBT was assessed in the same animal where the arterial thrombosis induced by mechanic damage was performed. In the anesthetized guinea pig, a standard cut was made with a nail clipper at the apex of the nail cuticle of the forelegs and the paw was kept in contact with the surface of 37° C. water into which the blood flowed. The bleeding time was defined as the time after cuticle transection when bleeding was completely stopped. In case of re-bleeding within two minutes the time of bleeding was added to the initial bleeding time. This procedure was performed simultaneously in triplicate immediately after the 40 minutes experimental thrombosis period. Results are expressed in fold-prolongation of the control group value.

The surgical blood loss (SBL) was also measured in the same animal immediately after the NCBT. The anesthetized guinea pig is place in ventral position, the neck was shaved and a median incision (length 40 to 50 mm, depth 5 mm) was made from the ears to the scapula with a surgical blade (AESCULAP BB 524). Immediately after the incision blood was soaked with a dental gauze roll (No 1-14 111 00, Ø 8 mm, length 40 mm, Internationale Verbandstoff Fabrik, Neuhausen, Switzerland) placed lengthways into the wound. Dental roll was weighted before and after its 5 minutes placement into the wound and the difference between the weights was defined as blood loss (in mg) per 5 minutes. The total blood loss assessed for 1 hour corresponds to the sum of the blood soaked by the 12 dental rolls placed in the wound within the 1 hour measurement period.

The animal was subsequently euthanized by i. v. injection of pentobarbital (100 mg/kg) and the liver was rapidly removed. One gram of liver was shock frozen in liquid nitrogen for the determination of FVII mRNA as described below.

### Plasma Assays

FVII levels in guinea pig plasma were determined by the use of a commercial chromogenic assay (BIOPHEN FVII kit; ref 221304, HYPHEN BioMed, France). FVII levels were expressed in percent of pretreatment levels. Prothrombin time (PT) used as a marker of the clotting and bleeding tendency was determined by using human recombinant human tissue factor (Dade Innovin, Dade Behring, Marburg, Germany) as activator and activated partial thromboplastin time (aPTT) was determined by using phospholipids as activator (Dade Actin, Dade Behring, Marburg, Germany). PT and aPTT were measured using an ACL3000^{plus} Coagulation Systems Analyzer and are expressed in fold prolongation of pretreatment values. Alanine aminotransferase (ALT) and aspartate aminotransferase (AST) were measured using a Hitachi 912 Automatic Analyser (Boehringer Mannheim, Germany) and ALT Kit no 10851132216, AST (Asat/Got) Kit no 10851124216, Roche Diagnostics, Switzerland).

Blood samples were also collected into EDTA for measurements of blood cell counts, platelets and hematocrit (Cobas Helios VET, F. Hoffmann-La Roche, Basel, Switzerland).

dsRNAs were formulated in LNP01 as described previously (Akinc, A. et al., Nature Biotech 2008, 26(5):561-9.). In addition, dsRNAs formulated in SNALP-L were tested. (Judge A. D. et al., J. Clinic. Invest. 2009, 119(3):661-73.).

Sequences of bDNA probes for determination of guinea pig Factor VII

| | | | SEQ |
|---|---|---|---|
| | | | ID |
| FPL Name | Function | Sequence | No. |
| cpoFak7 001 | CE | ggttcctccatgcattccgtTTTTTctcttggaaagaaagt | 380 |
| cpoFak7 002 | CE | ggcctcctcgaatgtgcatTTTTTctcttggaaagaaagt | 381 |
| cpoFak7 003 | CE | ggcaggtgcctccgttctTTTTTctcttggaaagaaagt | 382 |
| cpoFak7 004 | CE | ttcgggaggcagaagcagaTTTTTctcttggaaagaaagt | 383 |
| cpoFak7 005 | CE | cagttccggccgctgaagTTTTTctcttggaaagaaagt | 384 |
| cpoFak7 006 | CE | agtgcgctcctgtttgtctcaTTTTTctcttggaaagaaagt | 385 |
| cpoFak7 007 | LE | ggtggtcctgaggatctcccTTTTTaggcataggacccgtgtct | 386 |
| cpoFak7 008 | LE | cccagaactggttcgtcttctcTTTTTaggcataggacccgtgtct | 387 |
| cpoFak7 009 | LE | caccattctcattgtcacagatcagcTTTTTaggcataggacccgtgtct | 388 |
| cpoFak7 010 | LE | gcgcgtgtctcccttgcgTTTTTaggcataggacccgtgtct | 389 |
| cpoFak7 011 | LE | gcgtggcaccggcagatTTTTTaggcataggacccgtgtct | 390 |
| cpoFak7 012 | BL | tggtccccgtcagtatatgaag | 391 |
| cpoFak7 013 | BL | ggcaagggtttgaggcacac | 392 |
| cpoFak7 014 | BL | tgtacagccggaagtcgtctt | 393 |
| cpoFak7 015 | BL | gtcactgcagtactgctcacagc | 394 |

Sequences of bDNA probes for determination of rat GAPDH

| | | | SEQ |
|---|---|---|---|
| | | | ID |
| FPL Name | Function | Sequence | No. |
| rGAPD001 | CE | ccagcttcccattctcagccTTTTTctcttggaaagaaagt | 395 |
| rGAPD002 | CE | tctcgctcctggaagatggtTTTTTctcttggaaagaaagt | 396 |
| rGAPD003 | CE | cccatttgatgttagcgggaTTTTTctcttggaaagaaagt | 397 |
| rGAPD004 | CE | cggagatgatgacccttttggTTTTTctcttggaaagaaagt | 398 |
| rGAPD005 | LE | gatgggtttcccgttgatgaTTTTTaggcataggacccgtgtct | 399 |
| rGAPD006 | LE | gacatactcagcaccagcatcacTTTTTaggcataggacccgtgtct | 400 |
| rGAPD007 | LE | cccagccttctccatggtggTTTTTaggcataggacccgtgtct | 401 |
| rGAPD008 | BL | ttgactgtgccgttgaacttg | 402 |
| rGAPD009 | BL | tgaagacgccagtagactccac | 403 |
| rGAPD010 | BL | ccccacccttcaggtgagc | 404 |
| rGAPD011 | BL | ggcatcagcggaagggg | 405 |

FVII mRNA Measurement in Guinea Pig Liver Tissue:

FVII mRNA measurements were done from liver tissue using QuantiGene 1.0 branched DNA (bDNA) Assay Kit (Panomics, Fremont, Calif., USA, Cat-No: QG0004).

At necropsy 1-2 g liver tissue was snap frozen in liquid nitrogen. Frozen tissue was powderized with mortar and pistil on dry ice. 15-25 mg of tissue was transferred to a chilled 1.5 ml reaction tube, 1 ml 1:3 Lysis Mixture prediluted in MilliQ water and 3.3 μl Proteinase K (50 μg/μl) was added and tissue was lysed by several seconds ultrasound sonication at 30-50% power (HD2070, Bandelin, Berlin, Germany). Lysates were stored at −80° C. until analysis. For mRNA analysis lysate was thawed and Proteinase K digested for 15 min at 1000 rpm and 65° C. (Thermomixer comfort, Eppendorf, Hamburg, Germany). FVII and GAPDH mRNA levels were determined using QuantiGene 1.0 bDNA Assay Kit reagents and according to the manufacturer's recommendations. FVII expression was analyzed using 200 lysate and cavia porcellus FVII probeset and GAPDH expression was analyzed using 401 lysate and rattus norwegicus probesets shown to crossreact with guinea pig (sequences of probesets see below). Chemiluminescence signal at end of assay was measured in a Victor 2 Light luminescence counter (Perkin Elmer, Wiesbaden, Germany) as relative light units (RLU). FVII signal was divided by same lysate GAPDH signal and values depicted as FVII expression normalized to GAPDH.

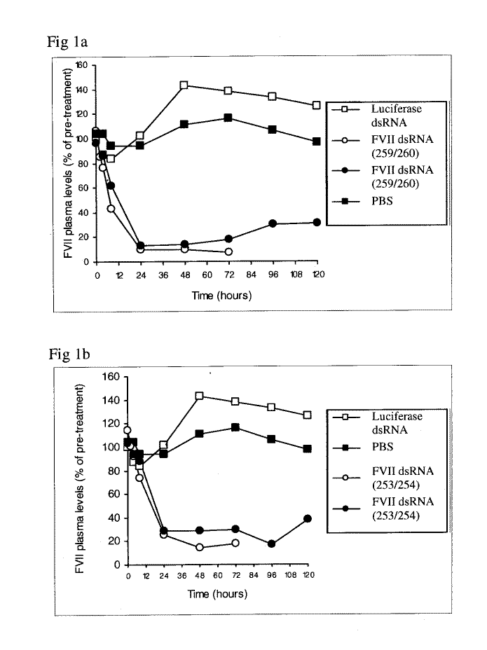

As example (FIG. 1), the time course of FVII plasma level was followed over 3 and 5 days after injection of FVII dsRNA comprising SEQ ID pairs 259/260 and FVII dsRNA comprising SEQ ID pairs 253/254 at 4 mg/kg in a LNP01 liposome formulation [lipid:dsRNA ratio (w/w)14:1, 96% entrapment, 80-85 nm size] into the guinea pig jugular vein. A maximal FVII knock down was achieved 24 hours post-injection lasting for at least 72 hours.

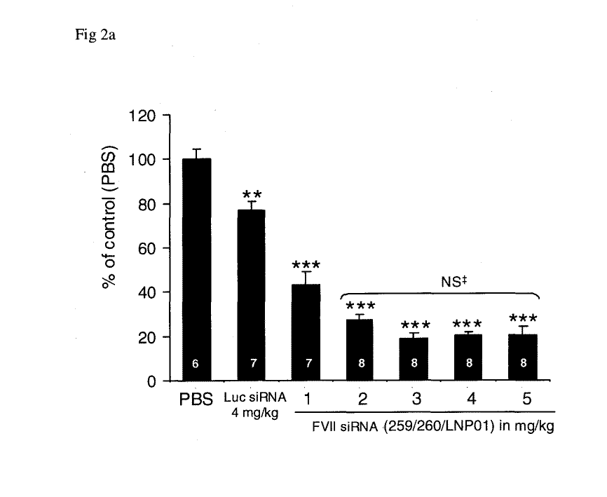

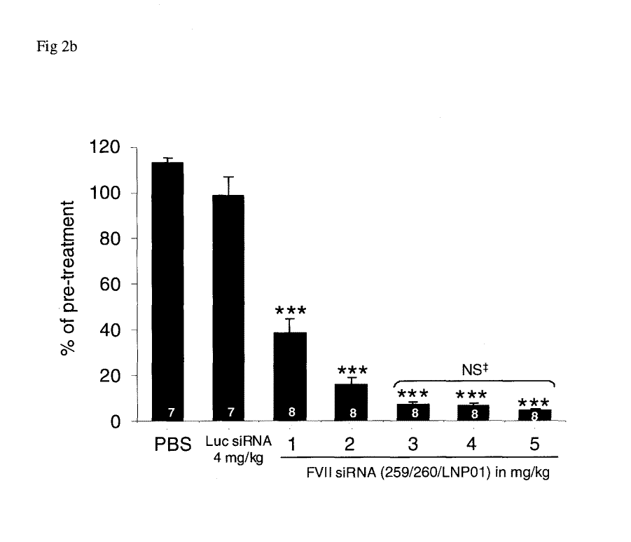

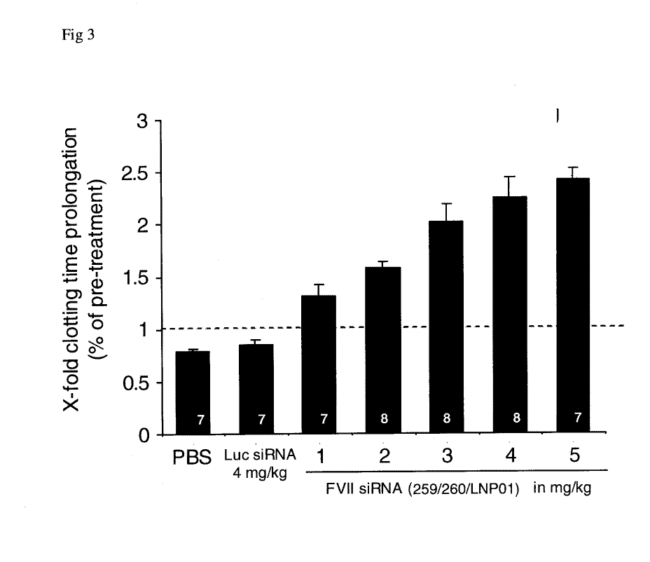

FVII dsRNA comprising SEQ ID pairs 259/260/LNP01 (1:14) was tested in the guinea pig arterial thrombosis model at 1, 2, 3, 4, 5 mg/kg, single i.v. dose. Phosphate buffered saline (PBS) and Luciferase dsRNA (SEQ ID pairs 411/412)/LNP01 (1:14) were used as controls. FVII mRNA levels in liver (FIG. 2a) and FVII zymogen levels in plasma (FIG. 2b) decreased in a dose dependent manner, while PT was prolonged accordingly (FIG. 3).

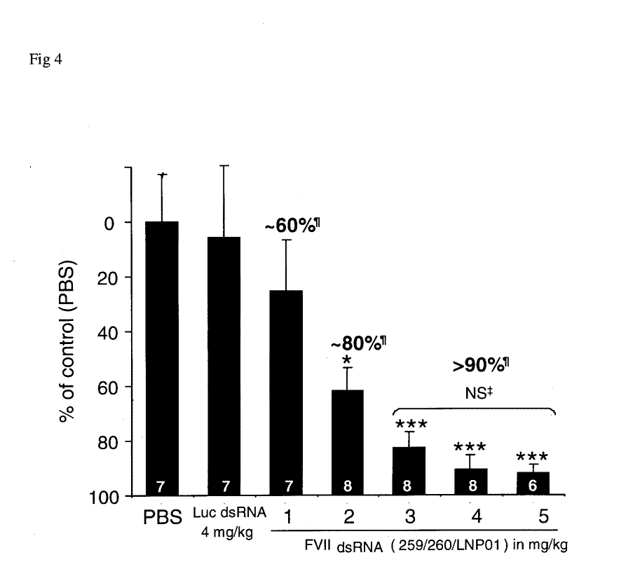

A FVII knock down in plasma superior to 80% was associated with a significant inhibition of thrombus formation in the guinea pig arterial thrombosis model. The observed IC50 was between 1 and 2 mg/kg of FVII dsRNA comprising SEQ ID pairs 259/260/LNP01 (1:14). At 3, 4, 5 mg/kg FVII dsRNA comprising SEQ ID pairs 259/260/LNP01 (1:14) a similar FVII plasma knock down (about 95%) and liver mRNA knock down (about 80%) was associated with similar antithrombotic effects (about 90% inhibition of thrombus formation) (FIG. 4).

1 mg/kg induced a 56% knock down of FVII mRNA in liver, a 62% knock down of FVII in plasma, prolonged PT by 1.3-fold, inhibited thrombin generation (peak height) by 4% and inhibited thrombus formation by about 26%.

2 mg/kg induced a 73% knock down of FVII mRNA in liver, a 84% knock down of FVII in plasma, prolonged PT by 1.6-fold, inhibited thrombin generation (peak height) by 22% and inhibited thrombus formation by about 62%.

3 mg/kg induced a 81% knock down of FVII mRNA in liver, a 93% knock down of FVII in plasma, prolonged PT by 2.0-fold, inhibited thrombin generation (peak height) by 27% and inhibited thrombus formation by about 82%.

4 mg/kg induced a 80% knock down of FVII mRNA in liver, a 93% knock down of FVII in plasma, prolonged PT by 2.3-fold, inhibited thrombin generation (peak height) by 43% and inhibited thrombus formation by about 91%.

5 mg/kg induced a 80% knock down of FVII mRNA in liver, a 95% knock down of FVII in plasma, prolonged PT by 2.4-fold, inhibited thrombin generation (peak height) by 40% and inhibited thrombus formation by about 92%.

Bleeding assessed by nail cuticle bleeding time and surgical blood loss was not significantly affected at the tested FVII dsRNA SEQ ID NOs pair 259/260/LNP01 (1:14) doses (1, 2, 3, 4, 5 mg/kg) suggesting that a normal haemostasis was maintained up to about 95% FVII knock down in plasma.

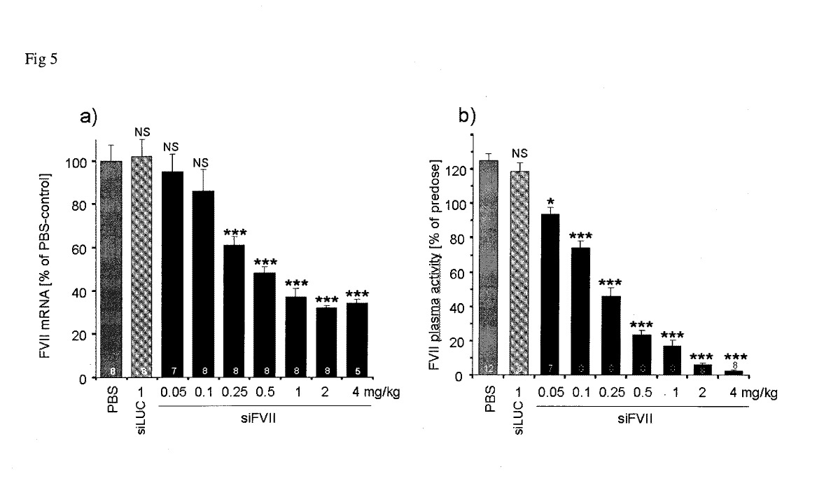

FIG. 5 shows the FVII mRNA levels in liver (FIG. 5a) and FVII zymogen levels in plasma (FIG. 5b) when FVII dsRNA comprising SEQ ID pairs 259/260 was formulated in SNALP-L.

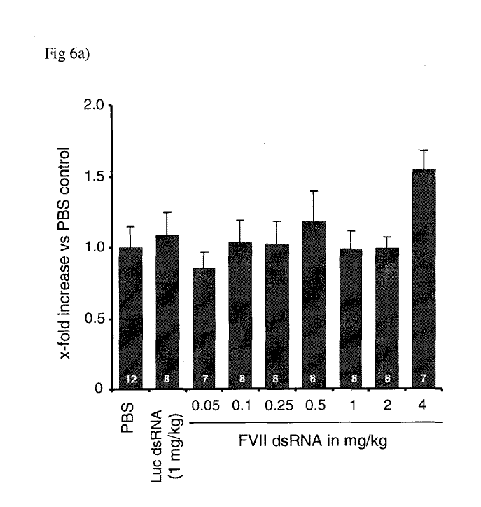

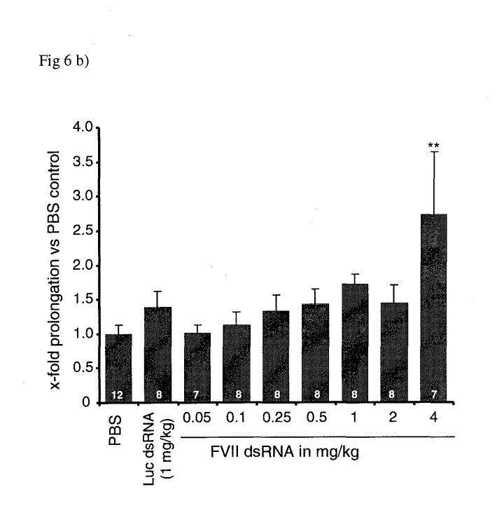

FIG. 6 shows the effect of FVII dsRNA on (a) surgical blood loss and (b) nail cuticle bleeding time in guinea pigs after i.v. injection of FVII dsRNA comprising Seq. ID pair 259/260 in a SNALP-L formulation (siFVII).

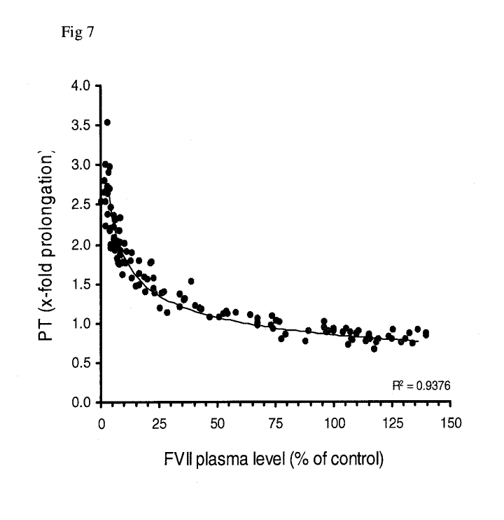

FIG. 7 shows the correlation between FVII activity in plasma and PT-prolongation. FVII activity decrease after iv injection of FVII dsRNA (combined data from FVII dsRNA formulated in LNP01 and SNALP-L) correlated well with FVII-dependent coagulation parameter PT.

In Vivo Effects of dsRNA Targeting FVII (Macaca fascicularis)

For the following studies a sterile formulation of dsRNA in lipid particles in isotonic buffer (“stable nucleic acid-lipid particles” (SNALP) technology, Tekmira Pharmaceuticals Corporation, Canada) were used.

Single Dose Titration Study in Monkeys (Macaca fascicularis)

Monkeys received single iv bolus injections of FVII dsRNA (Seq. IDs 19/20) ranging from 0.3 mg/kg to 10 mg/kg. Control groups received a 10 mg/kg high dose of Luciferase dsRNA (Seq. IDs 411/412) in order to discriminate between effects caused by the lipid particle and RNAi-mediated effects. Monkeys were sacrificed 48 hours after injection.

Pharmacological effect was monitored in plasma and liver. FVII activity and PT values were measured in plasma 24 hours and 48 hours after injection. FVII mRNA levels were measured in liver 48 hours after injection at the time of sacrifice.

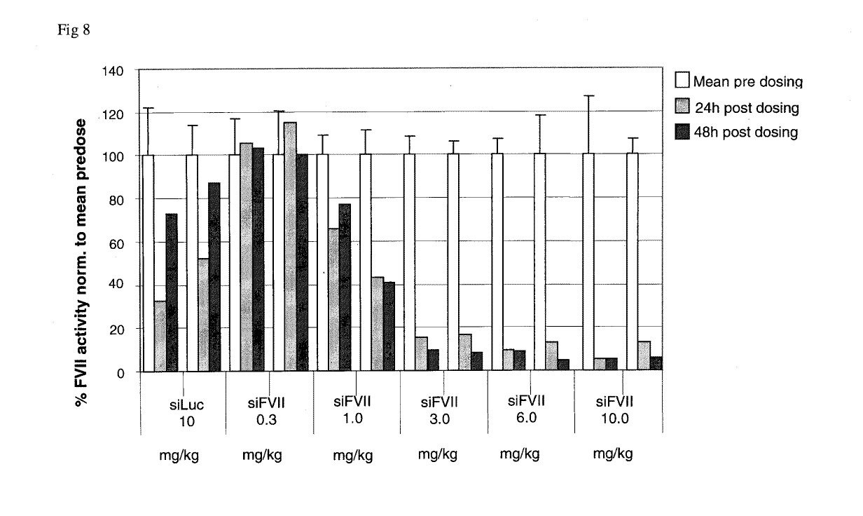

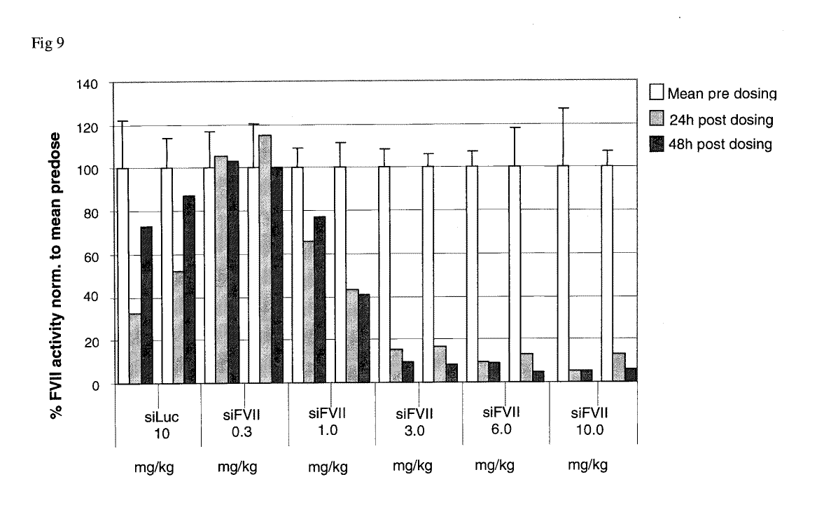

FVII dsRNA (Seq. IDs 19/20) treated groups showed a dose-dependent decrease in FVII activity of about 50% at 1 mg/kg of dsRNA and reached >90% decrease in FVII activity at 3 mg/kg of FVII dsRNA (Seq. IDs 19/20) at 24 and 48 hours after iv injection (FIG. 8). At doses of 6 mg/kg and 10 mg/kg, the decrease in FVII activity was similar to that seen at 3 mg/kg of FVII dsRNA (Seq. IDs 19/20). PT prolongation was observed starting at 3 mg/kg (FIG. 9). Additional prolongations in PT were observed as the dose was increased to 6 mg/kg and 10 mg/kg. PT prolongation was between 1.2-fold at 3 mg/kg and 1.4-fold at 10 mg/kg.

### Exploratory Study in Monkeys to Assess Duration of Effect and Repeated Dosing

Single and repeated doses were studied in male cynomolgous monkeys using FVII dsRNA (Seq. IDs 19/20). The study objectives were to gain further insight into the duration and kinetics of the pharmacological effect of FVII dsRNA (Seq. IDs 19/20), as well as to evaluate the safety and efficacy of multiple dosing.

Monkeys received either single or repeated doses of FVII dsRNA (Seq. IDs 19/20). The objective of single dosing was to examine duration of effect. Monkeys in the single dose groups received bolus injections of 3 mg/kg and 6 mg/kg of FVII dsRNA (Seq. IDs 19/20). A 6 mg/kg Luciferase dsRNA (Seq. IDs 411/412) group was used to control for dsRNA sequence-dependent silencing and to assess lipid particle related effects. The objective of repeated dosing was to study dose additivity and to identify a maximal tolerated dose, as defined by either lipid particle toxicity or potential bleeding issues due to exaggerated pharmacology. Monkeys in the two repeated dose groups were scheduled to receive three once weekly bolus injections of FVII dsRNA (Seq. IDs 19/20) at 3 mg/kg and 10 mg/kg.

As a follow-up to findings in single dose monkey study described above, a 3 mg/kg Luciferase dsRNA (Seq. IDs 411/412) female monkey group was included to further characterize lipid particle-mediated effects at a lower dose. Pharmacologic effects (FVII activity and PT) were monitored from plasma samples taken at multiple time points during the study and at the time of sacrifice.

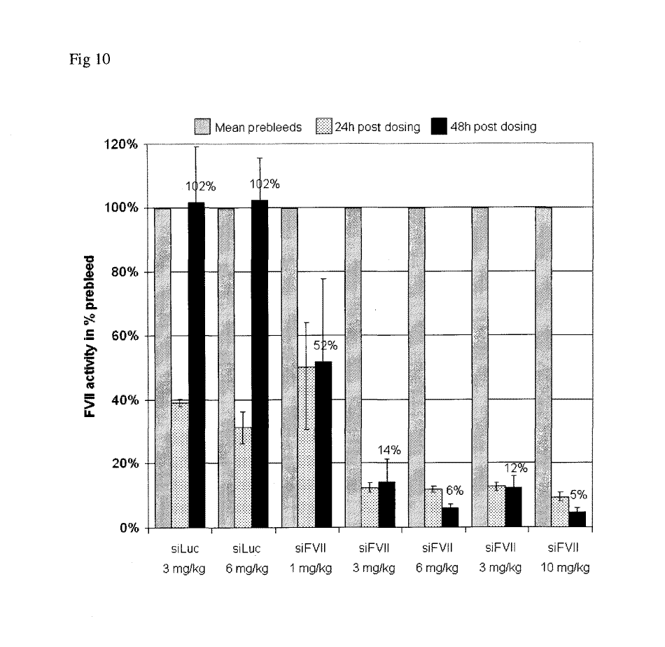

Compiled data for FVII activity at 24 hours and 48 hours were similar to data from the single dose study described above (FIG. 10). FVII dsRNA (Seq. IDs 19/20) reduced FVII activity by about 50% at 1 mg/kg and by about 85% to 95% at the 3, 6 and 10 mg/kg doses. Luciferase dsRNA control groups at 3 and 6 mg/kg confirmed the dsRNA lipid particle has a transient unspecific impact on FVII activity at 24 hours. Values returned to normal at 48 hours. Therefore, activity seen at 48 hours in the 3 and 6 mg/kg FVII dsRNA (Seq. IDs 19/20) groups can be fully attributed to the pharmacological activity of FVII dsRNA.

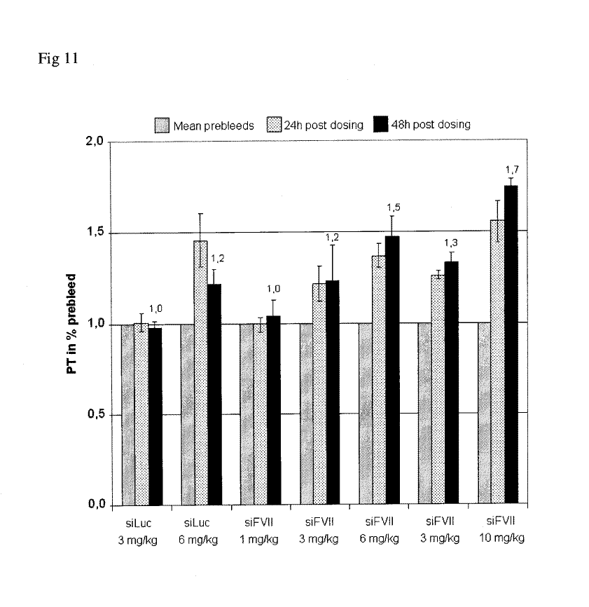

PT values are shown in FIG. 11. PT prolongation of 1.2-fold was observed at 3 mg/kg and increased in a dose-dependent manner to 1.7-fold at 10 mg/kg.

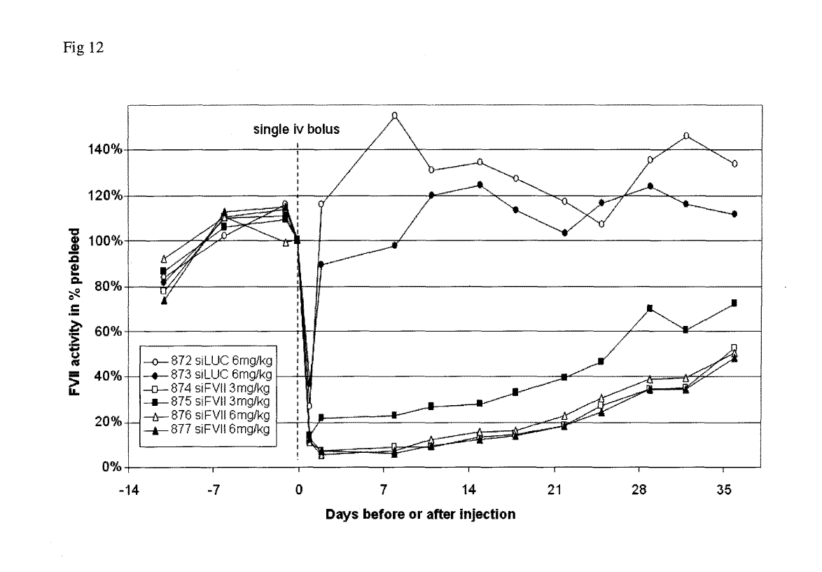

Duration of pharmacological effect in monkeys was about 6 weeks, based on extrapolation from FVII activity levels in plasma followed over >1 month (FIG. 12). Full reduction of FVII activity persisted for about 1 week after which FVII activity was progressively restored. Similar silencing kinetics were observed at 3 and 6 mg/kg, suggesting that there was no depot effect and that FVII dsRNA given at doses higher than required for simple full FVII activity inhibition does not necessarily prolong the pharmacological effect.

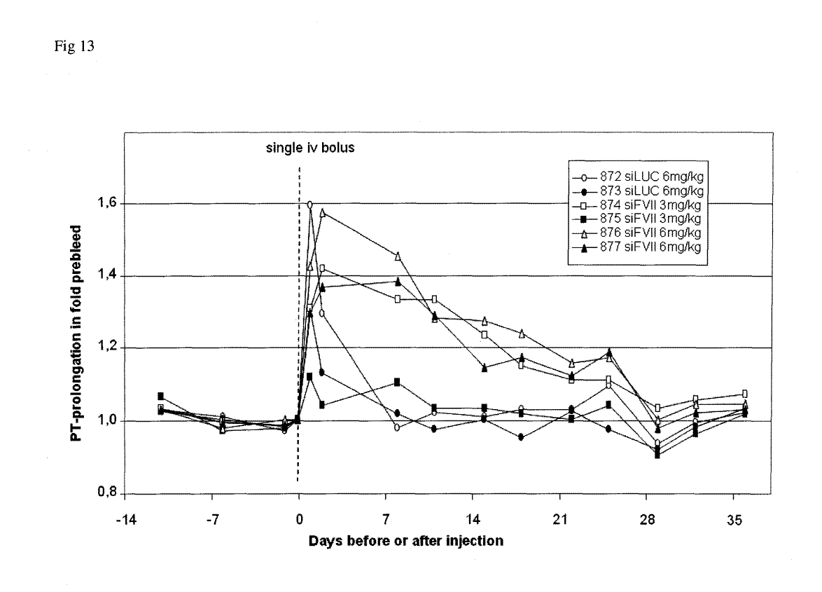

PT prolongation was seen for 4 weeks with the highest values in the first week after treatment, followed by a linear decline in weeks 2 to 4 (FIG. 13). Data indicate that >70% of FVII activity reduction was needed in order to see an effect on this FVII-dependent biomarker.

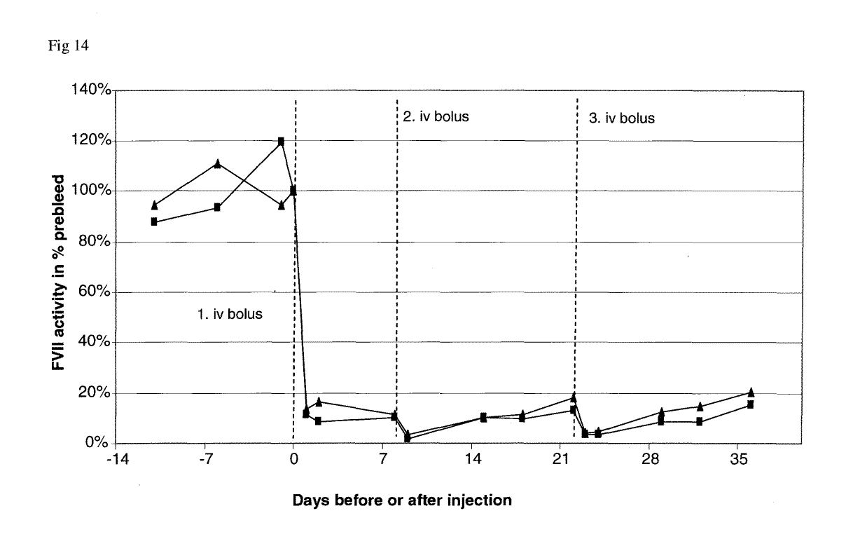

Multiple dosing at 3 mg/kg at once weekly intervals is shown in FIG. 14. Intervals between the second and third doses were widened from one week to two weeks in order to explore a steady state situation and to avoid exaggerated efficacy and toxicological effects. FVII activity data indicated that locking FVII levels in a steady state interval was feasible.

Dosing at 3 mg/kg at two or three week intervals appeared to be optimal to maintain an 80% to 95% FVII activity reduction. PT values can be kept in a 1.2- to 1.8-fold prolongation.

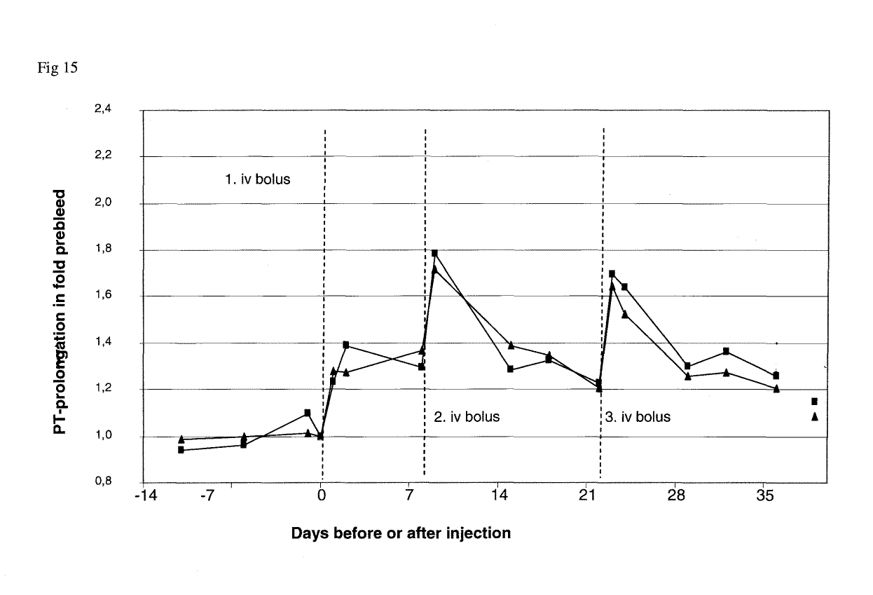

Dosing at 3 mg/kg in two or three week intervals seemed optimal to maintain an 80% to 95% FVII activity reduction. PT values can be kept in a 1.2- to 1.8-fold prolongation interval (FIG. 15), with marked PT peaks noted a few days after injection. These peaks were likely due to additive effects from pharmacological activity of FVII dsRNA and unspecific effect from the lipid particle.

In Vitro Off-Target Analysis of dsRNA Targeting Human FVII

The psiCHECK™-vector (Promega) contains two reporter genes for monitoring RNAi activity: a synthetic version of the Renilla luciferase (hRluc) gene and a synthetic firefly luciferase gene (hluc+). The firefly luciferase gene permits normalization of changes in Renilla luciferase expression to firefly luciferase expression. Renilla and firefly luciferase activities were measured using the Dual-Glo® Luciferase Assay System (Promega).

To use the psiCHECK™ vectors for analyzing off-target effects of the inventive dsRNAs, the predicted off-target sequence was cloned into the multiple cloning region located 3′ to the synthetic Renilla luciferase gene and its translational stop codon.

After cloning, the vector is transfected into a mammalian cell line, and subsequently cotransfected with dsRNAs targeting FVII. If the dsRNA effectively initiates the RNAi process on the target RNA of the predicted off-target, the fused Renilla target gene mRNA sequence will be degraded, resulting in reduced Renilla luciferase activity.

In Silico Off-Target Prediction

The human genome was searched by computer analysis for sequences homologous to the inventive dsRNAs. Homologous sequences that displayed less than 5 mismatches with the inventive dsRNAs were defined as a possible off-targets. Off-targets selected for in vitro off-target analysis are given in appended tables 8, 9 and 10.

Generation of psiCHECK Vectors Containing Predicted Off-Target Sequences

The strategy for analyzing off target effects for an siRNA lead candidate includes the cloning of the predicted off target sites into the psiCHECK2 Vector system (Dual Glo®-system, Promega, Braunschweig, Germany cat. No C8021) via XhoI and NotI restriction sites. Therefore, the off target site is extended with 10 nucleotides upstream and downstream of the siRNA target site. Additionally, a NheI restriction site is integrated to prove insertion of the fragment by restriction analysis.

The single-stranded oligonucleotides were annealed according to a standard protocol (e.g. protocol by Metabion) in a Mastercycler (Eppendorf) and then cloned into psiCHECK (Promega) previously digested with XhoI and NotI. Successful insertion was verified by restriction analysis with NheI and subsequent sequencing of the positive clones. The selected primer (Seq ID No. 761) for sequencing binds at position 1401 of vector psiCHECK. After clonal production the plasmids were analyzed by sequencing and than used in cell culture experiments.

Analysis of dsRNA Off-Target Effects

### Cell Culture:

Cos7 cells were obtained from Deutsche Sammlung für Mikroorganismen and Zellkulturen (DSMZ, Braunschweig, Germany, cat. No. ACC-60) and cultured in DMEM (Biochrom AG, Berlin, Germany, cat. No. F0435) supplemented to contain 10% fetal calf serum (FCS) (Biochrom AG, Berlin, Germany, cat. No. S0115), Penicillin 100 U/ml, and Streptomycin 100 μg/ml (Biochrom AG, Berlin, Germany, cat. No. A2213) and 2 mM L-Glutamine (Biochrom AG, Berlin, Germany, cat. No. K0283) as well as 12 μg/ml Natrium-bicarbonate at 37° C. in an atmosphere with 5% CO2 in a humidified incubator (Heraeus HERAcell, Kendro Laboratory Products, Langenselbold, Germany).

### Transfection and Luciferase Quantification:

For transfection with plasmids, Cos-7 cells were seeded at a density of 2.25×104 cells/well in 96-well plates and transfected directly. Transfection of plasmids was carried out with lipofectamine 2000 (Invitrogen GmbH, Karlsruhe, Germany, cat. No. 11668-019) as described by the manufacturer at a concentration of 50 ng/well. 4 hours after transfection, the medium was discarded and fresh medium was added.

The siRNAs were transfected in a concentration at 50 nM using lipofectamine 2000 as described above. 24 h after siRNA transfection the cells were lysed using Luciferase reagent described by the manufacturer (Dual-Glo™ Luciferase Assay system, Promega, Mannheim, Germany, cat. No. E2980) and Firefly and Renilla Luciferase were quantified according to the manufacturer's protocol. Renilla Luciferase protein levels were normalized to Firefly Luciferase levels.

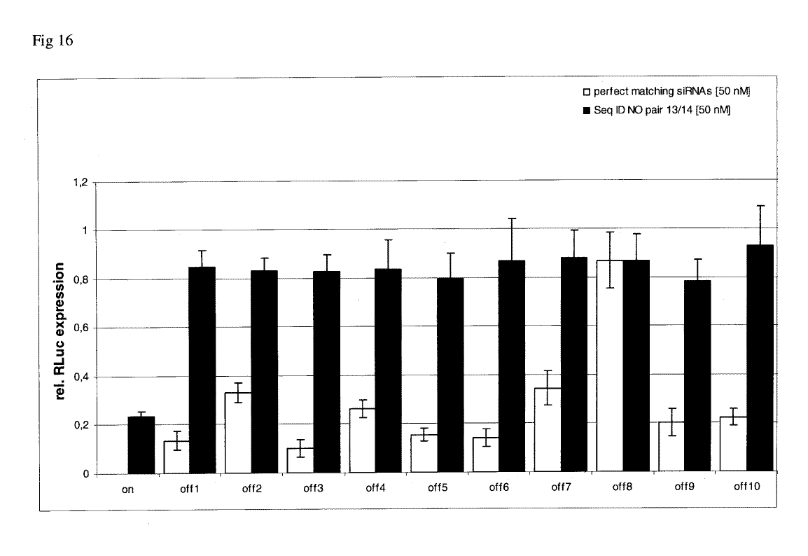

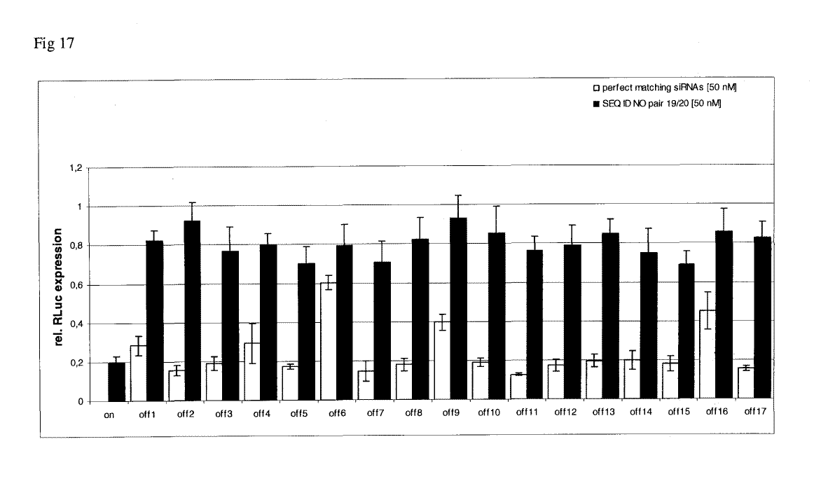

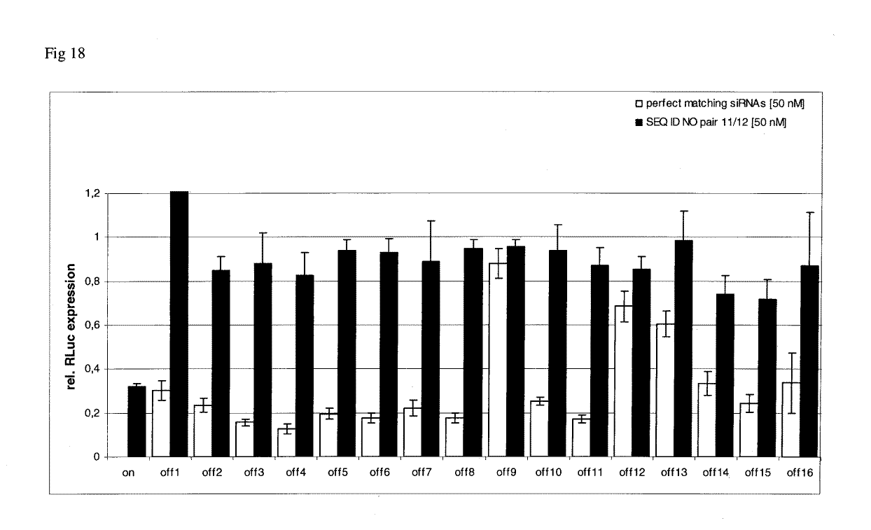

For each siRNA twelve individual data points were collected in three independent experiments. A siRNA unrelated to all target sites was used as a control to determine the relative Renilla Luciferase protein levels in siRNA treated cells. Results are given in FIGS. 16, 17 and 18.

Unless stated to the contrary, all ranges recited herein encompass all combinations and subcombinations included within that range limit. All patents and publications cited herein are hereby incorporated by reference in their entirety.

TABLE 1

Activity testing
with 30 nM dsRNA
in Huh7 cells

| | | | | | | |
|---|---|---|---|---|---|---|
| | | | | | mean % | |
| | SEQ ID | sense strand sequence | SEQ ID | antisense strand sequence | knock- | standard |
| Rank | NO | (5′-3′) | NO | (5′-3′) | down | deviation |

| | | | | | | |
|---|---|---|---|---|---|---|
| 1 | 1 | uucuGGuucuuAuccAuuAdTsdT | 2 | uAAUGGAuAAGAACcAGAAdTsdT | 78.33 | 4.79 |
| 2 | 3 | GAcAcAGAGAuGGAAuAGAdTsd | 4 | UCuAUUCcAUCUCUGUGUCdTsdT | 77.94 | 2.70 |
| | | T | | | | |
| 3 | 5 | GcAccAAAucccAuAuAuudTsdT | 6 | AAuAuAUGGGAUUUGGUGCdTsdT | 77.12 | 1.45 |
| 4 | 7 | GAAAAAuAccuAuucuAGAdTsdT | 8 | UCuAGAAuAGGuAUUUUUCdTsdT | 76.97 | 4.16 |
| 5 | 9 | AAAGccAAGGcuGcGucGAdTsdT | 10 | UCGACGcAGCCUUGGCUUUdTsdT | 76.56 | 5.91 |
| 6 | 11 | GAGAuAuGcAcAcAccGAudTsdT | 12 | AUCGGUGUGUGcAuAUCUCdTsdT | 75.33 | 8.71 |
| 7 | 13 | uGcAAAAGcucAuGcGcucdTsdT | 14 | GAGCGcAUGAGCUUUUGcAdTsdT | 73.14 | 4.03 |
| 8 | 15 | AcAcAucAGuGcAcAcGGAdTsdT | 16 | UCCGUGUGcACUGAUGUGUdTsdT | 73.13 | 5.46 |
| 9 | 17 | cuucGuGcGcuucucAuuGdTsdT | 18 | cAAUGAGAAGCGcACGAAGdTsdT | 71.90 | 4.98 |
| 10 | 19 | AGAuAuGcAcAcAcAcGGAdTsdT | 20 | UCCGUGUGUGUGcAuAUCUdTsdT | 70.17 | 11.58 |
| 11 | 21 | cGuGcGcuucucAuuGGucdTsdT | 22 | GACcAAUGAGAAGCGcACGdTsdT | 70.10 | 4.03 |
| 12 | 23 | AGcuucAcAAuAAAcGGcudTsdT | 24 | AGCCGUUuAUUGUGAAGCUdTsdT | 69.83 | 12.47 |
| 13 | 25 | cccAGcuucAcAAuAAAcGdTsdT | 26 | CGUUuAUUGUGAAGCUGGGdTsdT | 69.78 | 5.90 |
| 14 | 27 | GAcAGuAGAGGcAuGAAcAdTsdT | 28 | UGUUcAUGCCUCuACUGUCdTsdT | 69.41 | 8.07 |
| 15 | 29 | AGccAAGGcuGcGucGAAcdTsdT | 30 | GUUCGACGcAGCCUUGGCUdTsdT | 68.96 | 9.69 |
| 16 | 31 | GAGucAGGGAcAcAcGcAudTsdT | 32 | AUGCGUGUGUCCCUGACUCdTsdT | 68.83 | 12.65 |
| 17 | 33 | ccAAAuAucAcGGAGuAcAdTsdT | 34 | UGuACUCCGUGAuAUUUGGdTsdT | 68.71 | 10.97 |
| 18 | 35 | cGAuGcAcAcGcAcAuAGAdTsdT | 36 | UCuAUGUGCGUGUGcAUCGdTsdT | 68.60 | 9.16 |
| 19 | 37 | ccAuGcAuGGuGGcGAAuGdTsdT | 38 | cAUUCGCcACcAUGcAUGGdTsdT | 68.45 | 11.54 |
| 20 | 39 | GuGuGAAcGAGAAcGGcGGdTsdT | 40 | CCGCCGUUCUCGUUcAcACdTsdT | 67.94 | 6.82 |
| 21 | 41 | cuGcccGAAcGGAcGuucudTsdT | 42 | AGAACGUCCGUUCGGGcAGdTsdT | 67.50 | 12.65 |
| 22 | 43 | cuGGcAccAAAucccAuAudTsdT | 44 | AuAUGGGAUUUGGUGCcAGdTsdT | 67.20 | 6.38 |
| 23 | 45 | GGucAcAcAGAGAuAcGcAdTsdT | 46 | UGCGuAUCUCUGUGUGACCdTsdT | 67.08 | 5.80 |
| 24 | 47 | cGGAcGuucucuGAGAGGAdTsdT | 48 | UCCUCUcAGAGAACGUCCGdTsdT | 66.91 | 7.44 |
| 25 | 49 | uGuGcGcAcAcAcAGAuAudTsdT | 50 | AuAUCUGUGUGUGCGcAcAdTsdT | 65.71 | 9.51 |
| 26 | 51 | GcGcAcAcAcAccGAuGuAdTsdT | 52 | uAcAUCGGUGUGUGUGCGCdTsdT | 65.31 | 3.85 |
| 27 | 53 | AuGuGcGcAcAcAcAGAuAdTsdT | 54 | uAUCUGUGUGUGCGcAcAUdTsdT | 65.06 | 14.32 |
| 28 | 55 | GucAcAcAGAGAuAcGcAAdTsdT | 56 | UUGCGuAUCUCUGUGUGACdTsdT | 63.98 | 7.59 |
| 29 | 57 | GccAAuGcAcGcAcAcAucdTsdT | 58 | GAUGUGUGCGUGcAUUGGCdTsdT | 63.56 | 12.26 |
| 30 | 59 | uGAucuGuGuGAAcGAGAAdTsdT | 60 | UUCUCGUUcAcAcAGAUcAdTsdT | 63.16 | 10.08 |
| 31 | 61 | GcGGcccAcuGuuucGAcAdTsdT | 62 | UGUCGAAAcAGUGGGCCGCdTsdT | 63.08 | 4.43 |
| 32 | 63 | cAAuGcAcGcAcAcAucAGdTsdT | 64 | CUGAUGUGUGCGUGcAUUGdTsdT | 63.00 | 5.81 |
| 33 | 65 | cAcAccGAuGuGcGcAcAcdTsdl | 66 | GUGUGCGcAcAUCGGUGUGdTsdT | 62.75 | 6.95 |
| 34 | 67 | GcGGuuGuuuAGcucucAcdTsdT | 68 | GUGAGAGCuAAAcAACCGCdTsdT | 62.41 | 4.92 |
| 35 | 69 | AccAuGcAuGGuGGcGAAudTsdT | 70 | AUUCGCcACcAUGcAUGGUdTsdT | 62.40 | 2.52 |
| 36 | 71 | AcAucAGuGcAcAcGGAuGdTsdT | 72 | cAUCCGUGUGcACUGAUGUdTsdT | 61.32 | 11.78 |
| 37 | 73 | ucccAGcuucAcAAuAAAcdTsdT | 74 | GUUuAUUGUGAAGCUGGGAdTsdT | 61.25 | 8.36 |
| 38 | 75 | GAGAuuucAucAuGGucucdTsdT | 76 | GAGACcAUGAUGAAAUCUCdTsdT | 60.83 | 8.36 |
| 39 | 77 | GAAGGcGGuuGuuuAGcucdTsdT | 78 | GAGCuAAAcAACCGCCUUCdTsdT | 60.83 | 4.70 |
| 40 | 79 | ccucuGAAGGcGGuuGuuudTsdT | 80 | AAAcAACCGCCUUcAGAGGdTsdT | 59.75 | 10.31 |
| 41 | 81 | cuGuGuGAAcGAGAAcGGcdTsdT | 82 | GCCGUUCUCGUUcAcAcAGdTsdT | 59.72 | 9.37 |
| 42 | 83 | uGcccGAAcGGAcGuucucdTsdT | 84 | GAGAACGUCCGUUCGGGcAdTsdT | 59.38 | 10.16 |
| 43 | 85 | uGGcAccAAAucccAuAuAdTsdT | 86 | uAuAUGGGAUUUGGUGCcAdTsdT | 58.93 | 3.66 |
| 44 | 87 | AuAcGcAAAcAcAccGAuGdTsdT | 88 | cAUCGGUGUGUUUGCGuAUdTsdT | 58.64 | 5.63 |
| 45 | 89 | cuGuccucuGAAGGcGGuudTsdT | 90 | AACCGCCUUcAGAGGAcAGdTsdT | 56.93 | 2.72 |
| 46 | 91 | cAccAAGcGcuccuGucGGdTsdT | 92 | CCGAcAGGAGCGCUUGGUGdTsdT | 54.35 | 9.06 |
| 47 | 93 | ccAGcuucAcAAuAAAcGGdTsdT | 94 | CCGUUuAUUGUGAAGCUGGdTsdT | 54.31 | 16.87 |
| 48 | 95 | AuGccAAuGcAcGcAcAcAdTsdT | 96 | UGUGUGCGUGcAUUGGcAUdTsdT | 54.17 | 13.13 |
| 49 | 97 | cAcAcAucAGuGcAcAcGGdTsdT | 98 | CCGUGUGcACUGAUGUGUGdTsdT | 53.51 | 7.77 |
| 50 | 99 | GucAcGGAAGGuGGGAGAcdTsdT | 100 | GUCUCCcACCUUCCGUGACdTsdT | 53.12 | 18.87 |
| 51 | 101 | AcAcAGAGAuAcGcAAAcAdTsdT | 102 | UGUUUGCGuAUCUCUGUGUdTsdT | 52.43 | 17.87 |
| 52 | 103 | AcAuGccAAuGcAcGcAcAdTsdT | 104 | UGUGCGUGcAUUGGcAUGUdTsdT | 52.42 | 13.77 |
| 53 | 105 | GcAcGuAcGucccGGGcAcdTsdT | 106 | GUGCCCGGGACGuACGUGCdTsdT | 49.63 | 16.67 |
| 54 | 107 | GGGAGuGccAAGGuuGuccdTsdT | 108 | GGAcAACCUUGGcACUCCCdTsdT | 47.89 | 12.96 |
| 55 | 109 | uAuAcAcAuGGAuGcAcGcdTsdT | 110 | GCGUGcAUCcAUGUGuAuAdTsdT | 47.24 | 8.48 |
| 56 | 111 | GuccucuGAAGGcGGuuGudTsdT | 112 | AcAACCGCCUUcAGAGGACdTsdT | 45.77 | 22.39 |
| 57 | 113 | GcccAcuGuuucGAcAAAAdTsdT | 114 | UUUUGUCGAAAcAGUGGGCdTsdT | 45.70 | 6.79 |
| 58 | 115 | cAcGcAcAuAGAGAuAuGcdTsdT | 116 | GcAuAUCUCuAUGUGCGUGdTsdT | 45.21 | 8.13 |
| 59 | 117 | GccGGcGcGccAAcGcGuudTsdT | 118 | AACGCGUUGGCGCGCCGGCdTsdT | 44.57 | 9.51 |
| 60 | 119 | GcucAGAGAGuGGAcucGAdTsdT | 120 | UCGAGUCcACUCUCUGAGCdTsdT | 41.65 | 7.11 |
| 61 | 121 | ccucAGcGAGcAcGAcGGGdTsdT | 122 | CCCGUCGUGCUCGCUGAGGdTsdT | 41.20 | 15.50 |
| 62 | 123 | uucGuGcGcuucucAuuGGdTsdT | 124 | CcAAUGAGAAGCGcACGAAdTsdT | 40.29 | 17.48 |
| 63 | 125 | GAAAGccAAGGcuGcGucGdTsdT | 126 | CGACGcAGCCUUGGCUUUCdTsdT | 39.66 | 8.61 |
| 64 | 127 | GAccAGcuccAGuccuAuAdTsdT | 128 | uAuAGGACUGGAGCUGGUCdTsdT | 39.37 | 10.97 |
| 65 | 129 | uGcGcAcAcAcAccGAuGudTsdT | 130 | AcAUCGGUGUGUGUGCGcAdTsdT | 39.30 | 16.06 |
| 66 | 131 | AGAGAuuucAucAuGGucudTsdT | 132 | AGACcAUGAUGAAAUCUCUdTsdT | 39.17 | 11.17 |
| 67 | 133 | cAAAuAucAcGGAGuAcAudTsdT | 134 | AUGuACUCCGUGAuAUUUGdTsdT | 37.85 | 20.92 |
| 68 | 135 | AcGcAcAcAucAGuGcAcAdTsdT | 136 | UGUGcACUGAUGUGUGCGUdTsdT | 37.83 | 11.77 |
| 69 | 137 | cAccAccAAccAcGAcAucdTsdT | 138 | GAUGUCGUGGUUGGUGGUGdTsdT | 37.60 | 12.87 |
| 70 | 139 | uGGAcucGAuGccAucccudTsdT | 140 | AGGGAUGGcAUCGAGUCcAdTsdT | 37.34 | 12.02 |
| 71 | 141 | cucuGccuGcccGAAcGGAdTsdT | 142 | UCCGUUCGGGcAGGcAGAGdTsdT | 36.35 | 15.50 |
| 72 | 143 | uucuGuGccGGcuAcucGGdTsdT | 144 | CCGAGuAGCCGGcAcAGAAdTsdT | 35.73 | 15.70 |
| 73 | 145 | cAcGuAcGucccGGGcAccdTsdT | 146 | GGUGCCCGGGACGuACGUGdTsdT | 35.28 | 4.57 |
| 74 | 147 | ccucuGccuGcccGAAcGGdTsdT | 148 | CCGUUCGGGcAGGcAGAGGdTsdT | 35.27 | 27.41 |
| 75 | 149 | GcGcGccAAcGcGuuccuGdTsdT | 150 | cAGGAACGCGUUGGCGCGCdTsdT | 34.85 | 13.53 |
| 76 | 151 | GGcccAcuGuuucGAcAAAdTsdT | 152 | UUUGUCGAAAcAGUGGGCCdTsdT | 34.44 | 16.18 |
| 77 | 153 | AGAucuucAAGGAcGcGGAdTsdT | 154 | UCCGCGUCCUUGAAGAUCUdTsdT | 34.31 | 22.61 |
| 78 | 155 | AuGuAuuucucccuucGcudTsdT | 156 | AGCGAAGGGAGAAAuAcAUdTsdT | 34.06 | 12.94 |
| 79 | 157 | GAuAuGcAcAcAccGAuGudTsdT | 158 | AcAUCGGUGUGUGcAuAUCdTsdT | 33.67 | 28.74 |
| 80 | 159 | uAcuGcAGuGAccAcAcGGdTsdT | 160 | CCGUGUGGUcACUGcAGuAdTsdT | 33.51 | 30.90 |
| 81 | 161 | ccAGGGcuGcGcAAccGuGdTsdT | 162 | cACGGUUGCGcAGCCCUGGdTsdT | 32.82 | 11.52 |
| 82 | 163 | cAGuccuAuAucuGcuucudTsdT | 164 | AGAAGcAGAuAuAGGACUGdTsdT | 32.76 | 10.53 |
| 83 | 165 | ccuGcccGAAcGGAcGuucdTsdT | 166 | GAACGUCCGUUCGGGcAGGdTsdT | 32.72 | 13.15 |
| 84 | 167 | cAcGcAucAcuAAAuGcAAdTsdT | 168 | UUGcAUUuAGUGAUGCGUGdTsdT | 32.68 | 8.12 |
| 85 | 169 | uGcAcAcAccGAuGuGcGcdTsdT | 170 | GCGcAcAUCGGUGUGUGcAdTsdT | 32.33 | 10.62 |
| 86 | 171 | cAGcAcGuAcGucccGGGcdTsdT | 172 | GCCCGGGACGuACGUGCUGdTsdT | 32.07 | 17.69 |
| 87 | 173 | GuGcGcuucucAuuGGucAdTsdT | 174 | UGACcAAUGAGAAGCGcACdTsdT | 31.81 | 16.52 |
| 88 | 175 | AAcGGAcGuucucuGAGAGdTsdT | 176 | CUCUcAGAGAACGUCCGUUdTsdT | 30.84 | 15.48 |
| 89 | 177 | GAucuucAAGGAcGcGGAGdTsdT | 178 | CUCCGCGUCCUUGAAGAUCdTsdT | 30.18 | 13.77 |
| 90 | 179 | ccAuGGcAGGuccuGuuGudTsdT | 180 | AcAAcAGGACCUGCcAUGGdTsdT | 30.07 | 16.02 |
| 91 | 181 | cuAuGAAcuAcAGccGuGGdTsdT | 182 | CcACGGCUGuAGUUcAuAGdTsdT | 29.72 | 12.59 |
| 92 | 183 | uAcGcAAAcAcAccGAuGcdTsdT | 184 | GcAUCGGUGUGUUUGCGuAdTsdT | 29.71 | 9.91 |
| 93 | 185 | cAAGGcuGcGucGAAcuGudTsdT | 186 | AcAGUUCGACGcAGCCUUGdTsdT | 29.58 | 20.31 |
| 94 | 187 | AGAuAuGcAcAcAccGAuGdTsdT | 188 | cAUCGGUGUGUGcAuAUCUdTsdT | 29.53 | 15.27 |
| 95 | 189 | cuGcGucGAAcuGuccuGGdTsdT | 190 | CcAGGAcAGUUCGACGcAGdTsdT | 29.25 | 13.29 |
| 96 | 191 | AuGcGcAcAcAcAccGAuGdTsdT | 192 | cAUCGGUGUGUGUGCGcAUdTsdT | 29.13 | 15.20 |
| 97 | 193 | ucuGccuGcccGAAcGGAcdTsdT | 194 | GUCCGUUCGGGcAGGcAGAdTsdT | 28.99 | 15.85 |
| 98 | 195 | GAcuccGGcAAGcAcGGcudTsdT | 196 | AGCCGUGCUUGCCGGAGUCdTsdT | 28.80 | 13.81 |
| 99 | 197 | GAcGcuGGccuucGuGcGcdTsdT | 198 | GCGcACGAAGGCcAGCGUCdTsdT | 26.82 | 19.18 |
| 100 | 199 | cGcAcAcAcAccGAuGuAcdTsdT | 200 | GuAcAUCGGUGUGUGUGCGdTsdT | 26.59 | 23.69 |
| 101 | 201 | AGAuuucAucAuGGucuccdTsdT | 202 | GGAGACcAUGAUGAAAUCUdTsdT | 26.51 | 10.53 |
| 102 | 203 | AAGGcuGcGucGAAcuGucdTsdT | 204 | GAcAGUUCGACGcAGCCUUdTsdT | 26.31 | 21.28 |
| 103 | 205 | uGcGucuccuccGcAcAccdTsdT | 206 | GGUGUGCGGAGGAGACGcAdTsdT | 26.06 | 9.60 |
| 104 | 207 | AAuAAAcGGcuGcGucuccdTsdT | 208 | GGAGACGcAGCCGUUuAUUdTsdT | 25.90 | 22.77 |
| 105 | 209 | AuAuGcAcAcAcAcGGAuGdTsdT | 210 | cAUCCGUGUGUGUGcAuAUdTsdT | 25.65 | 22.14 |
| 106 | 211 | AAGGcGGuuGuuuAGcucudTsdT | 212 | AGAGCuAAAcAACCGCCUUdTsdT | 25.53 | 15.36 |
| 107 | 213 | AcGcAucAcuAAAuGcAAGdTsdT | 214 | CUUGcAUUuAGUGAUGCGUdTsdT | 25.50 | 11.60 |
| 108 | 215 | cuGccuGcccGAAcGGAcGdTsdT | 216 | CGUCCGUUCGGGcAGGcAGdTsdT | 25.49 | 13.11 |
| 109 | 217 | cGGcccAcuGuuucGAcAAdTsdT | 218 | UUGUCGAAAcAGUGGGCCGdTsdT | 24.64 | 17.25 |
| 110 | 219 | cAGGGcuGcGcAAccGuGGdTsdT | 220 | CcACGGUUGCGcAGCCCUGdTsdT | 24.26 | 17.44 |
| 111 | 221 | uGGucAcAcAGAGAuAcGcdTsdT | 222 | GCGuAUCUCUGUGUGACcAdTsdT | 23.56 | 20.90 |
| 112 | 223 | cuccuGucGGuGccAcGAGdTsdT | 224 | CUCGUGGcACCGAcAGGAGdTsdT | 23.34 | 17.00 |
| 113 | 225 | cAAGGAccAGcuccAGuccdTsdT | 226 | GGACUGGAGCUGGUCCUUGdTsdT | 23.30 | 21.67 |
| 114 | 227 | uucucAuuGGucAGcGGcudTsdT | 228 | AGCCGCUGACcAAUGAGAAdTsdT | 23.19 | 11.97 |
| 115 | 229 | GAGAucuucAAGGAcGcGGdTsdT | 230 | CCGCGUCCUUGAAGAUCUCdTsdT | 22.55 | 30.82 |
| 116 | 231 | AGAGAGuGGAcucGAuGccdTsdT | 232 | GGcAUCGAGUCcACUCUCUdTsdT | 22.25 | 17.36 |
| 117 | 233 | cucccAGuAcAucGAGuGGdTsdT | 234 | CcACUCGAUGuACUGGGAGdTsdT | 21.18 | 14.86 |
| 118 | 235 | AGucAGGGAcAcAcGcAucdTsdT | 236 | GAUGCGUGUGUCCCUGACUdTsdT | 19.19 | 21.42 |
| 119 | 237 | ccAucccuGcAGGGccGucdTsdT | 238 | GACGGCCCUGcAGGGAUGGdTsdT | 18.05 | 21.10 |
| 120 | 239 | AGucuucGuAAcccAGGAGdTsdT | 240 | CUCCUGGGUuACGAAGACUdTsdT | 16.08 | 14.86 |
| 121 | 241 | cAAGcGcuccuGucGGuGcdTsdT | 242 | GcACCGAcAGGAGCGCUUGdTsdT | 15.11 | 36.25 |
| 122 | 243 | GGuccucAcuGAccAuGuGdTsdT | 244 | cAcAUGGUcAGUGAGGACCdTsdT | 14.85 | 22.85 |
| 123 | 245 | AGGcuGcGucGAAcuGuccdTsdT | 246 | GGAcAGUUCGACGcAGCCUdTsdT | 11.71 | 12.50 |
| 124 | 247 | GGAcAcAcGcAucAcuAAAdTsdT | 248 | UUuAGUGAUGCGUGUGUCCdTsdT | 11.37 | 22.12 |
| 125 | 249 | uGcAcAcAcAccGAuGcuGdTsdT | 250 | cAGcAUCGGUGUGUGUGcAdTsdT | 11.11 | 20.53 |
| 126 | 251 | AcuGAAAuGAAcccucAcAdTsdT | 252 | UGUGAGGGUUcAUUUcAGUdTsdT | 6.68 | 22.51 |

TABLE 2

| | transfection 1 | | transfection 2 | | |
|---|---|---|---|---|---|
| SEQ ID | | max. % | | max. % | mean |
| NO pair | IC50 | knock-down | IC50 | knock-down | IC50 |
| 5/6 | 0.01 | 84.24 | 0.01 | 83.66 | 0.01 |
| 9/10 | 0.01 | 89.56 | 0.01 | 91.25 | 0.01 |
| 3/4 | 0.01 | 91.31 | 0.01 | 84.31 | 0.01 |
| 19/20 | 0.02 | 80.32 | 0.01 | 85.77 | 0.01 |
| 23/24 | 0.02 | 71.44 | 0.01 | 77.91 | 0.01 |
| 15/16 | 0.02 | 83.35 | 0.02 | 84.66 | 0.02 |
| 11/12 | 0.03 | 85.18 | 0.04 | 83.85 | 0.04 |
| 7/8 | 0.05 | 79.75 | 0.04 | 73.79 | 0.05 |
| 1/2 | 0.03 | 92.15 | 0.07 | 87.08 | 0.05 |
| 13/14 | 0.07 | 71.29 | 0.10 | 75.57 | 0.09 |
| 17/18 | 0.16 | 76.35 | 0.51 | 68.33 | 0.34 |
| 25/26 | 1.44 | 65.07 | 0.34 | 74.40 | 0.89 |
| 21/22 | 1.31 | 68.09 | 2.41 | 63.03 | 1.86 |

TABLE 3

| | Stability | | Stability | | | |
|---|---|---|---|---|---|---|
| | Cynomolgous | | Human | | | |
| | Plasma | | Serum | | | |
| SEQ | sense | antisense | sense | antisense | Human | |
| ID NO | strand | strand | strand | strand | PBMC assay | |
| pair | t½ [h] | t½ [h] | t½ [h] | t½ [h] | IFN-α | TNF-α |
| 13/14 | >24 | 17.40 | >24 | >24 | 0 | 0 |
| 3/4 | 11.52 | 10.48 | >24 | 2.62 | 0 | 0 |
| 11/12 | 15.59 | 4.79 | >24 | 1.91 | 0 | 0 |
| 15/16 | 8.71 | 4.30 | >24 | 1.75 | 0 | 0 |
| 19/20 | 8.52 | 7.52 | >24 | 1.59 | 0 | 0 |

TABLE 4

Activity testing
with 30 nM
dsRNA in Huh7
cells

| | | | | | | |
|---|---|---|---|---|---|---|
| | SEQ | | SEQ | | mean % | |
| | ID | sense strand sequence | ID | antisense strand sequence | knock- | standard |
| Rank | NO | (5′-3′) | NO | (5′-3′) | down | deviation |

| | | | | | | |
|---|---|---|---|---|---|---|
| 1 | 253 | cAGuuGAAuAuccAuGuGGdTsdT | 254 | CfCfACfAUfGGAUfAUfUfCfAACfUfGdTsdT | 75.34 | 5.99 |
| 2 | 255 | uGAGcAGuAcuGcAGuGAcdTsdT | 256 | GUfCfACfUfGCfAGUfACfUfGCfUfCfAdTsdT | 69.26 | 5.79 |
| 3 | 257 | GcuGuGAGcAGuAcuGcAGdTsdT | 258 | CfUfGCfAGUfACfUfGCfUfCfACfAGCfdTsdT | 67.81 | 9.81 |
| 4 | 259 | GGcuGuGAGcAGuAcuGcAdTsdT | 260 | UGcAGuACUGCUcAcAGCCdTsdT | 66.80 | 7.10 |
| 5 | 261 | GGcuGuGAGcAGuAcuGcAdTsdT | 262 | UfGCfAGUfACfUfGCfUfCfACfAGCfCfdTsdT | 64.57 | 9.73 |
| 6 | 263 | GuGAGcAGuAcuGcAGuGAdTsdT | 264 | UfCfACfUfGCfAGUfACfUfGCfUfCfACfdTsdT | 62.84 | 4.55 |
| 7 | 265 | cccAcAGuuGAAuAuccAudTsdT | 266 | AUfGGAUfAUfUfCfAACfUfGUfGGGdTsdT | 61.13 | 13.26 |
| 8 | 267 | cuGuGAGcAGuAcuGcAGudTsdT | 268 | ACfUfGCfAGUfACfUfGCfUfCfACfAGdTsdT | 60.08 | 7.98 |
| 9 | 269 | ccAcAGuuGAAuAuccAuGdTsdT | 270 | CfAUfGGAUfAUfUfCfAACfUfGUfGGdTsdT | 58.67 | 10.32 |
| 10 | 271 | AcAuGuucuGuGccGGcuAdTsdT | 272 | uAGCCGGcAcAGAAcAUGUdTsdT | 57.19 | 5.12 |
| 11 | 273 | ucGAGGAGGcccGGGAGAudTsdT | 274 | AUfCfUfCfCfCfGGGCfCfUfCfCfUfCfGAdTsdT | 56.43 | 7.95 |
| 12 | 275 | GAGcAGuAcuGcAGuGAccdTsdT | 276 | GGUfCfACfUfGCfAGUfACfUfGCfUfCfdTsdT | 55.60 | 16.65 |
| 13 | 277 | GcuGuGAGcAGuAcuGcAGdTsdT | 278 | CUGcAGuACUGCUcAcAGCdTsdT | 53.13 | 12.67 |
| 14 | 279 | cAcAGuuGAAuAuccAuGudTsdT | 280 | ACfAUfGGAUfAUfUfCfAACfUfGUfGdTsdT | 49.24 | 8.40 |
| 15 | 281 | cAuGuucuGuGccGGcuAcdTsdT | 282 | GUfAGCfCfGGCfACfAGAACfAUfGdTsdT | 48.62 | 11.09 |
| 16 | 283 | cGAGGAGGcccGGGAGAucdTsdT | 284 | GAUfCfUfCfCfCfGGGCfCfUfCfCfUfCfGdTsdT | 46.07 | 15.77 |
| 17 | 285 | cAuGuucuGuGccGGcuAcdTsdT | 286 | GuAGCCGGcAcAGAAcAUGdTsdT | 45.15 | 4.36 |
| 18 | 287 | cccAcAGuuGAAuAuccAudTsdT | 288 | AUGGAuAUUcAACUGUGGGdTsdT | 44.48 | 8.30 |
| 19 | 289 | AcAuGuucuGuGccGGcuAdTsdT | 290 | UfAGCfCfGGCfACfAGAACfAUfGUfdTsdT | 43.51 | 13.21 |
| 20 | 291 | uGuGAGcAGuAcuGcAGuGdTsdT | 292 | CfACfUfGCfAGUfACfUfGCfUfCfACfAdTsdT | 39.68 | 33.16 |
| 21 | 293 | cAGuuGAAuAuccAuGuGGdTsdT | 294 | CcAcAUGGAuAUUcAACUGdTsdT | 39.61 | 13.32 |
| 22 | 295 | GGccAGcuGcuGGAccGuGdTsdT | 296 | CfACfGGUfCfCfAGCfAGCfUfGGCfCfdTsdT | 38.69 | 8.56 |
| 23 | 297 | cAcAGuuGAAuAuccAuGudTsdT | 298 | AcAUGGAuAUUcAACUGUGdTsdT | 38.64 | 8.07 |
| 24 | 299 | GuGAGcAGuAcuGcAGuGAdTsdT | 300 | UcACUGcAGuACUGCUcACdTsdT | 36.29 | 15.73 |
| 25 | 301 | AuGuucuGuGccGGcuAcudTsdT | 302 | AGuAGCCGGcAcAGAAcAUdTsdT | 35.93 | 9.53 |
| 26 | 303 | ccAcAGuuGAAuAuccAuGdTsdT | 304 | cAUGGAuAUUcAACUGUGGdTsdT | 35.80 | 19.43 |
| 27 | 305 | AcAGuuGAAuAuccAuGuGdTsdT | 306 | cAcAUGGAuAUUcAACUGUdTsdT | 34.83 | 12.69 |
| 28 | 307 | AuGuucuGuGccGGcuAcudTsdT | 308 | AGUfAGCfCfGGCfACfAGAACfAUfdTsdT | 34.13 | 20.29 |
| 29 | 309 | cAGcuGcuGGAccGuGGcGdTsdT | 310 | CGCcACGGUCcAGcAGCUGdTsdT | 32.02 | 21.81 |
| 30 | 311 | GGccAGcuGcuGGAccGuGdTsdT | 312 | cACGGUCcAGcAGCUGGCCdTsdT | 30.63 | 8.05 |
| 31 | 313 | ucGAGGAGGcccGGGAGAudTsdT | 314 | AUCUCCCGGGCCUCCUCGAdTsdT | 29.81 | 16.64 |
| 32 | 315 | GccAGcuGcuGGAccGuGGdTsdT | 316 | CcACGGUCcAGcAGCUGGCdTsdT | 29.08 | 8.89 |
| 33 | 317 | GGGccAGcuGcuGGAccGudTsdT | 318 | ACGGUCcAGcAGCUGGCCCdTsdT | 28.24 | 8.84 |
| 34 | 319 | uucGAGGAGGcccGGGAGAdTsdT | 320 | UCUCCCGGGCCUCCUCGAAdTsdT | 27.35 | 12.20 |
| 35 | 321 | AGcuGcuGGAccGuGGcGcdTsdT | 322 | GCfGCfCfACfGGUfCfCfAGCfAGCfUfdTsdT | 25.51 | 15.62 |
| 36 | 323 | AGcuGcuGGAccGuGGcGcdTsdT | 324 | GCGCcACGGUCcAGcAGCUdTsdT | 25.39 | 15.89 |
| 37 | 325 | cAGcuGcuGGAccGuGGcGdTsdT | 326 | CfGCfCfACfGGUfCfCfAGCfAGCfUfGdTsdT | 24.50 | 26.65 |
| 38 | 327 | GGGccAGcuGcuGGAccGudTsdT | 328 | ACfGGUfCfCfAGCfAGCfUfGGCfCfCfdTsdT | 24.06 | 21.67 |
| 39 | 329 | uucGAGGAGGcccGGGAGAdTsdT | 330 | UfCfUfCfCfCfGGGCfCfUfCfGfUfCfGAAdTsdT | 19.57 | 18.56 |
| 40 | 331 | cGAGGAGGcccGGGAGAucdTsdT | 332 | GAUCUCCCGGGCCUCCUCGdTsdT | 19.41 | 19.02 |
| 41 | 333 | ccAGcuGcuGGAccGuGGcdTsdT | 334 | GCcACGGUCcAGcAGCUGGdTsdT | 17.14 | 19.93 |
| 42 | 335 | GccAGcuGcuGGAccGuGGdTsdT | 336 | CfCfACfGGUfCfCfAGCfAGCfUfGGCfdTsdT | 12.01 | 29.21 |
| 43 | 337 | ccAGcuGcuGGAccGuGGcdTsdT | 338 | GCfCfACfGGUfCfCfAGCfAGCfUfGGdTsdT | 7.55 | 38.04 |
| 44 | 339 | cuGcuGGAccGuGGcGccAdTsdT | 340 | UfGGCfGCfCfACfGGUfCfCfAGCfAGdTsdT | −9.45 | 69.84 |
| 45 | 341 | AcAGuuGAAuAuccAuGuGdTsdT | 342 | CfACfAUfGGAUfAUfUfCfAACfUfGUfdTsdT | −13.35 | 25.53 |
| 46 | 343 | GcuGcuGGAccGuGGcGccdTsdT | 344 | GGCfGCfCfACfGGUfCfCfAGCfAGCfdTsdT | −13.89 | 68.55 |
| 47 | 345 | GcuGcuGGAccGuGGcGccdTsdT | 346 | GGCGCcACGGUCcAGcAGCdTsdT | −26.66 | 55.10 |
| 48 | 347 | cuGcuGGAccGuGGcGccAdTsdT | 348 | UGGCGCcACGGUCcAGcAGdTsdT | −36.88 | 85.81 |

TABLE 5

| | Activity testing | | Activity testing for dose | | | | | | |
|---|---|---|---|---|---|---|---|---|---|
| | with 30 nM dsRNA | | response in Huh7 cells | | | | | | |
| | in Huh7 cells | | transfection 1 | | transfection 2 | | mean | Human PBMC | |
| SEQ ID | mean % knock- | standard | | max. % | | max. % | values | assay | |
| pair | down | deviation | IC50 | knock-down | IC50 | knock-down | mean IC50 | IFN-α | TNF-α |
| 259/260 | 66.80 | 7.10 | 0.05 | 79.04 | 0.02 | 80.98 | 0.03 | 0 | 0 |
| 253/254 | 75.34 | 5.99 | 0.07 | 85.79 | 0.04 | 90.11 | 0.05 | 0 | 0 |
| 255/256 | 69.26 | 5.79 | 0.13 | 75.88 | 0.06 | 76.15 | 0.10 | 0 | 0 |
| 257/258 | 67.81 | 9.81 | 0.14 | 84.18 | 0.06 | 87.62 | 0.10 | 0 | 0 |
| 267/268 | 60.08 | 7.98 | 0.78 | 67.67 | 0.03 | 61.90 | 0.40 | 0 | 0 |
| 261/262 | 64.57 | 9.73 | 0.71 | 58.68 | 0.11 | 80.58 | 0.41 | 0 | 0 |
| 265/266 | 61.13 | 13.26 | 1.04 | 60.19 | 0.27 | 54.96 | 0.66 | 0 | 0 |
| 263/264 | 62.84 | 4.55 | 21.82 | 51.55 | 0.15 | 57.54 | 10.99 | 0 | 0 |

TABLE 6

| SEQ | | SEQ | |
|---|---|---|---|
| ID | sense strand sequence | ID | antisense strand |
| NO | (5′-3′) | NO | sequence (5′-3′) |
| 413 | UUCUGGUUCUUAUCCAUUATT | 414 | UAAUGGAUAAGAACCAGAATT |
| 415 | GACACAGAGAUGGAAUAGATT | 416 | UCUAUUCCAUCUCUGUGUCTT |
| 417 | GCACCAAAUCCCAUAUAUUTT | 418 | AAUAUAUGGGAUUUGGUGCTT |
| 419 | GAAAAAUACCUAUUCUAGATT | 420 | UCUAGAAUAGGUAUUUUUCTT |
| 421 | AAAGCCAAGGCUGCGUCGATT | 422 | UCGACGCAGCCUUGGCUUUTT |
| 423 | GAGAUAUGCACACACCGAUTT | 424 | AUCGGUGUGUGCAUAUCUCTT |
| 425 | UGCAAAAGCUCAUGCGCUCTT | 426 | GAGCGCAUGAGCUUUUGCATT |
| 427 | ACACAUCAGUGCACACGGATT | 428 | UCCGUGUGCACUGAUGUGUTT |
| 429 | CUUCGUGCGCUUCUCAUUGTT | 430 | CAAUGAGAAGCGCACGAAGTT |
| 431 | AGAUAUGCACACACACGGATT | 432 | UCCGUGUGUGUGCAUAUCUTT |
| 433 | CGUGCGCUUCUCAUUGGUCTT | 434 | GACCAAUGAGAAGCGCACGTT |
| 435 | AGCUUCACAAUAAACGGCUTT | 436 | AGCCGUUUAUUGUGAAGCUTT |
| 437 | CCCAGCUUCACAAUAAACGTT | 438 | CGUUUAUUGUGAAGCUGGGTT |
| 439 | GACAGUAGAGGCAUGAACATT | 440 | UGUUCAUGCCUCUACUGUCTT |
| 441 | AGCCAAGGCUGCGUCGAACTT | 442 | GUUCGACGCAGCCUUGGCUTT |
| 443 | GAGUCAGGGACACACGCAUTT | 444 | AUGCGUGUGUCCCUGACUCTT |
| 445 | CCAAAUAUCACGGAGUACATT | 446 | UGUACUCCGUGAUAUUUGGTT |
| 447 | CGAUGCACACGCACAUAGATT | 448 | UCUAUGUGCGUGUGCAUCGTT |
| 449 | CCAUGCAUGGUGGCGAAUGTT | 450 | CAUUCGCCACCAUGCAUGGTT |
| 451 | GUGUGAACGAGAACGGCGGTT | 452 | CCGCCGUUCUCGUUCACACTT |
| 453 | CUGCCCGAACGGACGUUCUTT | 454 | AGAACGUCCGUUCGGGCAGTT |
| 455 | CUGGCACCAAAUCCCAUAUTT | 456 | AUAUGGGAUUUGGUGCCAGTT |
| 457 | GGUCACACAGAGAUACGCATT | 458 | UGCGUAUCUCUGUGUGACCTT |
| 459 | CGGACGUUCUCUGAGAGGATT | 460 | UCCUCUCAGAGAACGUCCGTT |
| 461 | UGUGCGCACACACAGAUAUTT | 462 | AUAUCUGUGUGUGCGCACATT |
| 463 | GCGCACACACACCGAUGUATT | 464 | UACAUCGGUGUGUGUGCGCTT |
| 465 | AUGUGCGCACACACAGAUATT | 466 | UAUCUGUGUGUGCGCACAUTT |
| 467 | GUCACACAGAGAUACGCAATT | 468 | UUGCGUAUCUCUGUGUGACTT |
| 469 | GCCAAUGCACGCACACAUCTT | 470 | GAUGUGUGCGUGCAUUGGCTT |
| 471 | UGAUCUGUGUGAACGAGAATT | 472 | UUCUCGUUCACACAGAUCATT |
| 473 | GCGGCCCACUGUUUCGACATT | 474 | UGUCGAAACAGUGGGCCGCTT |
| 475 | CAAUGCACGCACACAUCAGTT | 476 | CUGAUGUGUGCGUGCAUUGTT |
| 477 | CACACCGAUGUGCGCACACTT | 478 | GUGUGCGCACAUCGGUGUGTT |
| 479 | GCGGUUGUUUAGCUCUCACTT | 480 | GUGAGAGCUAAACAACCGCTT |
| 481 | ACCAUGCAUGGUGGCGAAUTT | 482 | AUUCGCCACCAUGCAUGGUTT |
| 483 | ACAUCAGUGCACACGGAUGTT | 484 | CAUCCGUGUGCACUGAUGUTT |
| 485 | UCCCAGCUUCACAAUAAACTT | 486 | GUUUAUUGUGAAGCUGGGATT |
| 487 | GAGAUUUCAUCAUGGUCUCTT | 488 | GAGACCAUGAUGAAAUCUCTT |
| 489 | GAAGGCGGUUGUUUAGCUCTT | 490 | GAGCUAAACAACCGCCUUCTT |
| 491 | CCUCUGAAGGCGGUUGUUUTT | 492 | AAACAACCGCCUUCAGAGGTT |
| 493 | CUGUGUGAACGAGAACGGCTT | 494 | GCCGUUCUCGUUCACACAGTT |
| 495 | UGCCCGAACGGACGUUCUCTT | 496 | GAGAACGUCCGUUCGGGCATT |
| 497 | UGGCACCAAAUCCCAUAUATT | 498 | UAUAUGGGAUUUGGUGCCATT |
| 499 | AUACGCAAACACACCGAUGTT | 500 | CAUCGGUGUGUUUGCGUAUTT |
| 501 | CUGUCCUCUGAAGGCGGUUTT | 502 | AACCGCCUUCAGAGGACAGTT |
| 503 | CACCAAGCGCUCCUGUCGGTT | 504 | CCGACAGGAOCGCUUGGUGTT |
| 505 | CCAGCUUCACAAUAAACGGTT | 506 | CCGUUUAUUGUGAAGCUGGTT |
| 507 | AUGCCAAUGCACGCACACATT | 508 | UGUGUGCGUGCAUUGGCAUTT |
| 509 | CACACAUCAGUGCACACGGTT | 510 | CCGUGUGCACUGAUGUGUGTT |
| 511 | GUCACGGAAGGUGGGAGACTT | 512 | GUCUCCCACCUUCCGUGACTT |
| 513 | ACACAGAGAUACGCAAACATT | 514 | UGUUUGCGUAUCUCUGUGUTT |
| 515 | ACAUGCCAAUGCACGCACATT | 516 | UGUGCGUGCAUUGGCAUGUTT |
| 517 | GCACGUACGUCCCGGGCACTT | 518 | GUGCCCGGGACGUACGUGCTT |
| 519 | GGGAGUGCCAAGGUUGUCCTT | 520 | GGACAACCUUGGCACUCCCTT |
| 521 | UAUACACAUGGAUGCACGCTT | 522 | GCGUGCAUCCAUGUGUAUATT |
| 523 | GUCCUCUGAAGGCGGUUGUTT | 524 | ACAACCGCCUUCAGAGGACTT |
| 525 | GCCCACUGUUUCGACAAAATT | 526 | UUUUGUCGAAACAGUGGGCTT |
| 527 | CACGCACAUAGAGAUAUGCTT | 528 | GCAUAUCUCUAUGUGCGUGTT |
| 529 | GCCGGCGCGCCAACGCGUUTT | 530 | AACGCGUUGGCGCGCCGGCTT |
| 531 | GCUCAGAGAGUGGACUCGATT | 532 | UCGAGUCCACUCUCUGAGCTT |
| 533 | CCUCAGCGAGCACGACGGGTT | 534 | CCCGUCGUGCUCGCUGAGGTT |
| 535 | UUCGUGCGCUUCUCAUUGGTT | 536 | CCAAUGAGAAGCGCACGAATT |
| 537 | GAAAGCCAAGGCUGCGUCGTT | 538 | CGACGCAGCCUUGGCUUUCTT |
| 539 | GACCAGCUCCAGUCCUAUATT | 540 | UAUAGGACUGGAGCUGGUCTT |
| 541 | UGCGCACACACACCGAUGUTT | 542 | ACAUCGGUGUGUGUGCGCATT |
| 543 | AGAGAUUUCAUCAUGGUCUTT | 544 | AGACCAUGAUGAAAUCUCUTT |
| 545 | CAAAUAUCACGGAGUACAUTT | 546 | AUGUACUCCGUGAUAUUUGTT |
| 547 | ACGCACACAUCAGUGCACATT | 548 | UGUGCACUGAUGUGUGCGUTT |
| 549 | CACCACCAACCACGACAUCTT | 550 | GAUGUCGUGGUUGGUGGUGTT |
| 551 | UGGACUCGAUGCCAUCCCUTT | 552 | AGGGAUGGCAUCGAGUCCATT |
| 553 | CUCUGCCUGCCCGAACGGATT | 554 | UCCGUUCGGGCAGGCAGAGTT |
| 555 | UUCUGUGCCGGCUACUCGGTT | 556 | CCGAGUAGCCGGCACAGAATT |
| 557 | CACGUACGUCCCGGGCACCTT | 558 | GGUGCCCGGGACGUACGUGTT |
| 559 | CCUCUGCCUGCCCGAACGGTT | 560 | CCGUUCGGGCAGGCAGAGGTT |
| 561 | GCGCGCCAACGCGUUCCUGTT | 562 | CAGGAACGCGUUGGCGCGCTT |
| 563 | GGCCCACUGUUUCGACAAATT | 564 | UUUGUCGAAACAGUGGGCCTT |
| 565 | AGAUCUUCAAGGACGCGGATT | 566 | UCCGCGUCCUUGAAGAUCUTT |
| 567 | AUGUAUUUCUCCCUUCGCUTT | 568 | AGCGAAGGGAGAAAUACAUTT |
| 569 | GAUAUGCACACACCGAUGUTT | 570 | ACAUCGGUGUGUGCAUAUCTT |
| 571 | UACUGCAGUGACCACACGGTT | 572 | CCGUGUGGUCACUGCAGUATT |
| 573 | CCAGGGCUGCGCAACCGUGTT | 574 | CACGGUUGCGCAGCCCUGGTT |
| 575 | CAGUCCUAUAUCUGCUUCUTT | 576 | AGAAGCAGAUAUAGGACUGTT |
| 577 | CCUGCCCGAACGGACGUUCTT | 578 | GAACGUCCGUUCGGGCAGGTT |
| 579 | CACGCAUCACUAAAUGCAATT | 580 | UUGCAUUUAGUGAUGCGUGTT |
| 581 | UGCACACACCGAUGUGCGCTT | 582 | GCGCACAUCGGUGUGUGCATT |
| 583 | CAGCACGUACGUCCCGGGCTT | 584 | GCCCGGGACGUACGUGCUGTT |
| 585 | GUGCGCUUCUCAUUGGUCATT | 586 | UGACCAAUGAGAAGCGCACTT |
| 587 | AACGGACGUUCUCUGAGAGTT | 588 | CUCUCAGAGAACGUCCGUUTT |
| 589 | GAUCUUCAAGGACGCGGAGTT | 590 | CUCCGCGUCCUUGAAGAUCTT |
| 591 | CCAUGGCAGGUCCUGUUGUTT | 592 | ACAACAGGACCUGCCAUGGTT |
| 593 | CUAUGAACUACAGCCGUGGTT | 594 | CCACGGCUGUAGUUCAUAGTT |
| 595 | UACGCAAACACACCGAUGCTT | 596 | GCAUCGGUGUGUUUGCGUATT |
| 597 | CAAGGCUGCGUCGAACUGUTT | 598 | ACAGUUCGACGCAGCCUUGTT |
| 599 | AGAUAUGCACACACCGAUGTT | 600 | CAUCGGUGUGUGCAUAUCUTT |
| 601 | CUGCGUCGAACUGUCCUGGTT | 602 | CCAGGACAGUUCGACGCAGTT |
| 603 | AUGCGCACACACACCGAUGTT | 604 | CAUCGGUGUGUGUGCGCAUTT |
| 605 | UCUGCCUGCCCGAACGGACTT | 606 | GUCCGUUCGGGCAGGCAGATT |
| 607 | GACUCCGGCAAGCACGGCUTT | 608 | AGCCGUGCUUGCCGGAGUCTT |
| 609 | GACGCUGGCCUUCGUGCGCTT | 610 | GCGCACGAAGGCCAGCGUCTT |
| 611 | CGCACACACACCGAUGUACTT | 612 | GUACAUCGGUGUGUGUGCGTT |
| 613 | AGAUUUCAUCAUGGUCUCCTT | 614 | GGAGACCAUGAUGAAAUCUTT |
| 615 | AAGGCUGCGUCGAACUGUCTT | 616 | GACAGUUCGACGCAGCCUUTT |
| 617 | UGCGUCUCCUCCGCACACCTT | 618 | GGUGUGCGGAGGAGACGCATT |
| 619 | AAUAAACGGCUGCGUCUCCTT | 620 | GGAGACGCAGCCGUUUAUUTT |
| 621 | AUAUGCACACACACGGAUGTT | 622 | CAUCCGUGUGUGUGCAUAUTT |
| 623 | AAGGCGGUUGUUUAGCUCUTT | 624 | AGAGCUAAACAACCGCCUUTT |
| 625 | ACGCAUCACUAAAUGCAAGTT | 626 | CUUGCAUUUAGUGAUGCGUTT |
| 627 | CUGCCUGCCCGAACGGACGTT | 628 | CGUCCGUUCGGGCAGGCAGTT |
| 629 | CGGCCCACUGUUUCGACAATT | 630 | UUGUCGAAACAGUGGGCCGTT |
| 631 | CAGGGCUGCGCAACCGUGGTT | 632 | CCACGGUUGCGCAGCCCUGTT |
| 633 | UGGUCACACAGAGAUACGCTT | 634 | GCGUAUCUCUGUGUGACCATT |
| 635 | CUCCUGUCGGUGCCACGAGTT | 636 | CUCGUGGCACCGACAGGAGTT |
| 637 | CAAGGACCAGCUCCAGUCCTT | 638 | GGACUGGAGCUGGUCCUUGTT |
| 639 | UUCUCAUUGGUCAGCGGCUTT | 640 | AGCCGCUGACCAAUGAGAATT |
| 641 | GAGAUCUUCAAGGACGCGGTT | 642 | CCGCGUCCUUGAAGAUCUCTT |
| 643 | AGAGAGUGGACUCGAUGCCTT | 644 | GGCAUCGAGUCCACUCUCUTT |
| 645 | CUCCCAGUACAUCGAGUGGTT | 646 | CCACUCGAUGUACUGGGAGTT |
| 647 | AGUCAGGGACACACGCAUCTT | 648 | GAUGCGUGUGUCCCUGACUTT |
| 649 | CCAUCCCUGCAGGGCCGUCTT | 650 | GACGGCCCUGCAGGGAUGGTT |
| 651 | AGUCUUCGUAACCCAGGAGTT | 652 | CUCCUGGGUUACGAAGACUTT |
| 653 | CAAGCGCUCCUGUCGGUGCTT | 654 | GCACCGACAGGAGCGCUUGTT |
| 655 | GGUCCUCACUGACCAUGUGTT | 656 | CACAUGGUCAGUGAGGACCTT |
| 657 | AGGCUGCGUCGAACUGUCCTT | 658 | GGACAGUUCGACGCAGCCUTT |
| 659 | GGACACACGCAUCACUAAATT | 660 | UUUAGUGAUGCGUGUGUCCTT |
| 661 | UGCACACACACCGAUGCUGTT | 662 | CAGCAUCGGUGUGUGUGCATT |
| 663 | ACUGAAAUGAACCCUCACATT | 664 | UGUGAGGGUUCAUUUCAGUTT |

TABLE 7

| SEQ | | SEQ | |
|---|---|---|---|
| ID | sense strand sequence | ID | antisense strand |
| NO | (5′-3′) | NO | sequence (5′-3′) |
| 665 | CAGUUGAAUAUCCAUGUGGTT | 666 | CCACAUGGAUAUUCAACUGTT |
| 667 | UGAGCAGUACUGCAGUGACTT | 668 | GUCACUGCAGUACUGCUCATT |
| 669 | GCUGUGAGCAGUACUGCAGTT | 670 | CUGCAGUACUGCUCACAGCTT |
| 671 | GGCUGUGAGCAGUACUGCATT | 672 | UGCAGUACUGCUCACAGCCTT |
| 673 | GGCUGUGAGCAGUACUGCATT | 674 | UGCAGUACUGCUCACAGCCTT |
| 675 | GUGAGCAGUACUGCAGUGATT | 676 | UCACUGCAGUACUGCUCACTT |
| 677 | CCCACAGUUGAAUAUCCAUTT | 678 | AUGGAUAUUCAACUGUGGGTT |
| 679 | CUGUGAGCAGUACUGCAGUTT | 680 | ACUGCAGUACUGCUCACAGTT |
| 681 | CCACAGUUGAAUAUCCAUGTT | 682 | CAUGGAUAUUCAACUGUGGTT |
| 683 | ACAUGUUCUGUGCCGGCUATT | 684 | UAGCCGGCACAGAACAUGUTT |
| 685 | UCGAGGAGGCCCGGGAGAUTT | 686 | AUCUCCCGGGCCUCCUCGATT |
| 687 | GAGCAGUACUGCAGUGACCTT | 688 | GGUCACUGCAGUACUGCUCTT |
| 689 | GCUGUGAGCAGUACUGCAGTT | 690 | CUGCAGUACUGCUCACAGCTT |
| 691 | CACAGUUGAAUAUCCAUGUTT | 692 | ACAUGGAUAUUCAACUGUGTT |
| 693 | CAUGUUCUGUGCCGGCUACTT | 694 | GUAGCCGGCACAGAACAUGTT |
| 695 | CGAGGAGGCCCGGGAGAUCTT | 696 | GAUCUCCCGGGCCUCCUCGTT |
| 697 | CAUGUUCUGUGCCGGCUACTT | 698 | GUAGCCGGCACAGAACAUGTT |
| 699 | CCCACAGUUGAAUAUCCAUTT | 700 | AUGGAUAUUCAACUGUGGGTT |
| 701 | ACAUGUUCUGUGCCGGCUATT | 702 | UAGCCGGCACAGAACAUGUTT |
| 703 | UGUGAGCAGUACUGCAGUGTT | 704 | CACUGCAGUACUGCUCACATT |
| 705 | CAGUUGAAUAUCCAUGUGGTT | 706 | CCACAUGGAUAUUCAACUGTT |
| 707 | GGCCAGCUGCUGGACCGUGTT | 708 | CACGGUCCAGCAGCUGGCCTT |
| 709 | CACAGUUGAAUAUCCAUGUTT | 710 | ACAUGGAUAUUCAACUGUGTT |
| 711 | GUGAGCAGUACUGCAGUGATT | 712 | UCACUGCAGUACUGCUCACTT |
| 713 | AUGUUCUGUGCCGGCUACUTT | 714 | AGUAGCCGGCACAGAACAUTT |
| 715 | CCACAGUUGAAUAUCCAUGTT | 716 | CAUGGAUAUUCAACUGUGGTT |
| 717 | ACAGUUGAAUAUCCAUGUGTT | 718 | CACAUGGAUAUUCAACUGUTT |
| 719 | AUGUUCUGUGCCGGCUACUTT | 720 | AGUAGCCGGCACAGAACAUTT |
| 721 | CAGCUGCUGGACCGUGGCGTT | 722 | CGCCACGGUCCAGCAGCUGTT |
| 723 | GGCCAGCUGCUGGACCGUGTT | 724 | CACGGUCCAGCAGCUGGCCTT |
| 725 | UCGAGGAGGCCCGGGAGAUTT | 726 | AUCUCCCGGGCCUCCUCGATT |
| 727 | GCCAGCUGCUGGACCGUGGTT | 728 | CCACGGUCCAGCAGCUGGCTT |
| 729 | GGGCCAGCUGCUGGACCGUTT | 730 | ACGGUCCAGCAGCUGGCCCTT |
| 731 | UUCGAGGAGGCCCGGGAGATT | 732 | UCUCCCGGGCCUCCUCGAATT |
| 733 | AGCUGCUGOACCGUGGCGCTT | 734 | GCGCCACGGUCCAGCAGCUTT |
| 735 | AGCUGCUGGACCGUGGCGCTT | 736 | GCGCCACGGUCCAGCAGCUTT |
| 737 | CAGCUGCUGGACCGUGGCGTT | 738 | CGCCACGGUCCAGCAGCUGTT |
| 739 | GGGCCAGCUGCUGGACCGUTT | 740 | ACGGUCCAGCAGCUGGCCCTT |
| 741 | UUCGAGGAGGCCCGGGAGATT | 742 | UCUCCCGGGCCUCCUCGAATT |
| 743 | CGAGGAGGCCCGGGAGAUCTT | 744 | GAUCUCCCGGGCCUCCUCGTT |
| 745 | CCAGCUGCUGGACCGUGGCTT | 746 | GCCACGGUCCAGCAGCUGGTT |
| 747 | GCCAGCUGCUGGACCGUGGTT | 748 | CCACGGUCCAGCAGCUGGCTT |
| 749 | CCAGCUGCUGGACCGUGGCTT | 750 | GCCACGGUCCAGCAGCUGGTT |
| 751 | CUGCUGGACCGUGGCGCCATT | 752 | UGGCGCCACGGUCCAGCAGTT |
| 753 | ACAGUUGAAUAUCCAUGUGTT | 754 | CACAUGGAUAUUCAACUGUTT |
| 755 | GCUGCUGGACCGUGGCGCCTT | 756 | GGCGCCACGGUCCAGCAGCTT |
| 757 | GCUGCUGGACCGUGGCGCCTT | 758 | GGCGCCACGGUCCAGCAGCTT |
| 759 | CUGCUGGACCGUGGCGCCATT | 760 | UGGCGCCACGGUCCAGCAGTT |

TABLE 8

| | | | | | Pos. from | |
|---|---|---|---|---|---|---|
| | | | Specificity | Number | 5′ end of | |
| | Accession | Description | score | mismatches | as | Region |
| Anti- | | | | | | |
| sense | | | | | | |
| ON | NM_000131.3 | Homo sapiens coagulation factor VII (serum | 0.00 | 0 | | CDS |
| | | prothrombin conversion accelerator) (F7), transcript | | | | |
| | | variant 1, mRNA | | | | |
| OFF-1 | NM_016260.2 | Homo sapiens IKAROS family zinc finger 2 (Helios) | 11.00 | 4 | 1 3 17 19 | CDS |
| | | (IKZF2), transcript variant 1, mRNA | | | | |
| OFF-2 | NM_002214.2 | Homo sapiens integrin, beta 8 (ITGB8), mRNA | 11.00 | 2 | 5 12 | CDS |
| OFF-3 | NM_173798.2 | Homo sapiens zinc finger, CCHC domain containing 12 | 11.00 | 4 | 1 7 17 19 | CDS |
| | | (ZCCHC12), mRNA | | | | |
| OFF-4 | XM_001716016.1 | PREDICTED: Homo sapiens hypothetical protein | 11.25 | 3 | 1 5 9 | CDS |
| | | LOC100129238 (LOC100129238), mRNA | | | | |
| OFF-5 | NM_001085437.1 | Homo sapiens chromosome 2 open reading frame 54 | 12.00 | 5 | 1 5 13 17 | 3UTR |
| | | (C2orf54), transcript variant 1, mRNA | | | 19 | |
| OFF-6 | XM_001723437.1 | PREDICTED: Homo sapiens H2B histone family, | 12.00 | 5 | 1 4 14 17 | 3UTR |
| | | member M (H2BFM), mRNA | | | 19 | |
| OFF-7 | NM_025248.2 | Homo sapiens SNAP25-interacting protein (SNIP), | 12.20 | 5 | 1 4 10 15 | 3UTR |
| | | mRNA | | | 19 | |
| OFF-8 | NM_001080421.1 | Homo sapiens unc-13 homolog A (C, elegans) | 12.20 | 3 | 2 10 18 | 3UTR |
| | | (UNC13A), mRNA | | | | |
| Sense | | | | | | |
| OFF-9 | NM_207372.1 | Homo sapiens SH2 domain containing 4B (SH2D4B), | 2.20 | 4 | 1 11 15 19 | 3UTR |
| | | mRNA | | | | |
| OFF- | NM_016368.3 | Homo sapiens myo-inositol 1-phosphate synthase A1 | 11.00 | 3 | 5 13 19 | CDS |
| 10 | | (ISYNA1), mRNA | | | | |

TABLE 9

| | | | | Number | Pos. from | |
|---|---|---|---|---|---|---|
| | | | Specificity | mis- | 5′ end of | |
| | Accession | Description | score | matches | as | Region |
| Anti- | | | | | | |
| sense | | | | | | |
| ON | NM_000131.3 | Homo sapiens coagulation factor VII (serum prothrombin | 0.0 | 0 | | 3UTR |
| | | conversion accelerator) (F7), transcript variant 1, mRNA | | | | |
| OFF-1 | XM_001720803.1 | PREDICTED: Homo sapiens hypothetical protein | 2.0 | 3 | 16 18 19 | 3UTR |
| | | LOC100129836 (LOC100129836), mRNA | | | | |
| OFF-2 | NM_021572.4 | Homo sapiens ectonucleotide | 3.0 | 3 | 13 16 18 | 3UTR |
| | | pyrophosphatase/phosphodiesterase 5 (putative function) | | | | |
| | | (ENPP5), mRNA | | | | |
| OFF-3 | NM_020798.1 | Homo sapiens ubiquitin specific peptidase 35 (USP35), mRNA | 3.2 | 5 | 1 11 12 16 | 3UTR |
| | | | | | 19 | |
| OFF-4 | NM_017644.3 | Homo sapiens kelch-like 24 (Drosophila) (KLHL24), mRNA | 3.3 | 4 | 1 9 12 17 | 3UTR |
| OFF-5 | NM_020154.2 | Homo sapiens chromosome 15 open reading frame 24 | 3.5 | 5 | 1 8 15 18 | 3UTR |
| | | (C15orf24), mRNA | | | 19 | |
| OFF-6 | NM_002903.2 | Homo sapiens recoverin (RCVRN), mRNA | 12.0 | 4 | 1 2 16 18 | 3UTR |
| OFF-7 | NM_013272.2 | Homo sapiens solute carrier organic anion transporter family, | 12.0 | 3 | 2 16 18 | 3UTR |
| | | member 3A1 (SLCO3A1), mRNA | | | | |
| OFF-8 | NM_020248.2 | Homo sapiens catenin, beta interacting protein 1 (CTNNBIP1), | 12.0 | 3 | 2 15 18 | 3UTR |
| | | transcript variant 1, mRNA | | | | |
| OFF-9 | NM_001083909.1 | Homo sapiens G protein-coupled receptor 123 (GPR123), | 12.0 | 3 | 2 15 18 | 3UTR |
| | | mRNA | | | | |
| OFF-10 | NM_024779.3 | Homo sapiens phosphatidylinositol-5-phosphate 4-kinase, | 12.0 | 4 | 1 2 16 17 | 3UTR |
| | | type II, gamma (PIP4K2C), mRNA | | | | |
| OFF-11 | NM_017824.4 | Homo sapiens membrane-associated ring finger (C3HC4) 5 | 12.2 | 4 | 1 3 10 17 | 3UTR |
| | | (MARCH5), mRNA | | | | |
| OFF-12 | NM_138731.3 | Homo sapiens mirror-image polydactyly 1 (MIPOL1), mRNA | 12.0 | 3 | 3 16 18 | 3UTR |
| OFF-13 | NM_153711.2 | Homo sapiens family with sequence similarity 26, member E | 12.0 | 3 | 3 13 18 | 3UTR |
| | | (FAM26E), mRNA | | | | |
| Sense | | | | | | |
| OFF-14 | NM_001012756.1 | Homo sapiens zinc finger protein 260 (ZNF260), mRNA | 12.5 | 3 | 4 8 18 | 3UTR |
| OFF-15 | NM_000991.3 | Homo sapiens ribosomal protein L28 (RPL28), mRNA | 11.00 | 4 | 1 7 12 19 | 3UTR |
| OFF-16 | XM_001719251.1 | PREDICTED: Homo sapiens hypothetical protein | 11.00 | 2 | 4 17 | CDS |
| | | LOC100132440 (LOC100132440), mRNA | | | | |
| OFF-17 | NM_016356.3 | Homo sapiens doublecortin domain containing 2 (DCDC2), | 11.00 | 3 | 2 16 19 | 3UTR |
| | | mRNA | | | | |

TABLE 10

| | | | | Number | Pos. from | |
|---|---|---|---|---|---|---|
| | | | Specificity | mis- | 5′ end of | |
| | Accession | Description | score | matches | as | Region |
| Anti- | | | | | | |
| sense | | | | | | |
| ON | NM_000131.3 | Homo sapiens coagulation factor VII (serum prothrombin | 0.00 | 0 | | 3UTR |
| | | conversion accelerator) (F7), transcript variant 1, mRNA | | | | |
| OFF-1 | NM_176863.1 | Homo sapiens proteasome (prosome, macropain) activator | 11.00 | 4 | 1 4 15 19 | 3UTR |
| | | subunit 3 (PA28 gamma; Ki) (PSME3), transcript variant 2, mRNA | | | | |
| OFF-2 | NM_018109.3 | Homo sapiens PAP associated domain containing 1 (PAPD1), | 11.00 | 4 | 1 4 14 19 | 3UTR |
| | | mRNA | | | | |
| OFF-3 | XR_040759.1 | PREDICTED: Homo sapiens misc_RNA (LOC401296), miscRNA | 11.00 | 4 | 1 3 14 19 | CDS |
| OFF-4 | NM_005245.3 | Homo sapiens FAT tumor suppressor homolog 1 (Drosophila) | 11.00 | 4 | 1 5 16 19 | CDS |
| | | (FAT), mRNA | | | | |
| OFF-5 | NM_001470.2 | Homo sapiens gamma-aminobutyric acid (GABA) B receptor, 1 | 11.20 | 4 | 1 3 11 19 | CDS |
| | | (GABBR1), transcript variant 1, mRNA | | | | |
| OFF-6 | NM_021161.3 | Homo sapiens potassium channel, subfamily K, member 10 | 12.00 | 5 | 1 4 14 17 | 3UTR |
| | | (KCNK10), transcript variant 1, mRNA | | | 19 | |
| OFF-7 | NM_021007.2 | Homo sapiens sodium channel, voltage-gated, type II, alpha | 12.00 | 5 | 1 4 15 17 | 3UTR |
| | | subunit (SCN2A), transcript variant 1, mRNA | | | 19 | |
| OFF-8 | NM_014755.1 | Homo sapiens SERTA domain containing 2 (SERTAD2), mRNA | 12.00 | 4 | 3 15 17 19 | 3UTR |
| OFF-9 | NM_031231.3 | Homo sapiens N-terminal EF-hand calcium binding protein 3 | 12.00 | 5 | 1 6 14 15 | 3UTR |
| | | (NECAB3), transcript variant 1, mRNA | | | 19 | |
| OFF-10 | NM_006076.4 | Homo sapiens HIV-1 Rev binding protein-like (HRBL), mRNA | 12.00 | 5 | 1 3 12 17 | 3UTR |
| | | | | | 19 | |
| OFF-11 | NM_182944.2 | Homo sapiens ninein (GSK3B interacting protein) (NIN), transcript variant | 12.00 | 4 | 1 3 15 17 | 3UTR |
| | | 1, mRNA | | | | |
| OFF-12 | NM_006045.1 | Homo sapiens ATPase, class II, type 9A (ATP9A), mRNA | 12.00 | 5 | 1 2 15 16 | 3UTR |
| | | | | | 19 | |
| OFF-13 | NM_014319.3 | Homo sapiens LEM domain containing 3 (LEMD3), mRNA | 12.00 | 5 | 1 4 12 17 | 3UTR |
| | | | | | 19 | |
| Sense | | | | | | |
| OFF-14 | XM_001715761.1 | PREDICTED: Homo sapiens hypothetical protein LOC100132931 | 2.00 | 4 | 1 16 17 19 | CDS |
| | | (LOC100132931), mRNA | | | | |
| OFF-15 | NM_015270.3 | Homo sapiens adenylate cyclase 6 (ADCY6), transcript variant 1, | 2.00 | 4 | 1 15 16 19 | 3UTR |
| | | mRNA | | | | |
| OFF-16 | NM_015428.1 | Homo sapiens zinc finger protein 473 (ZNF473), transcript variant | 11.00 | 4 | 1 3 16 19 | 3UTR |
| | | 1, mRNA | | | | |

1. A double-stranded ribonucleic acid molecule capable of inhibiting the expression of Factor VII gene in vitro by at least 70%.

2. A double-stranded ribonucleic acid molecule of claim 1, wherein said double-stranded ribonucleic acid molecule comprises a sense strand and an antisense strand, the antisense strand being at least partially complementary to the sense strand, whereby the sense strand comprises a sequence, which has an identity of at least 90% to at least a portion of an mRNA encoding Factor VII, wherein said sequence is (i) located in the region of complementarity of said sense strand to said antisense strand; and (ii) wherein said sequence is less than 30 nucleotides in length.

3. A double-stranded ribonucleic acid molecule of claim 1, comprising nucleotides 1-19 of SEQ ID Nos: 413, 414, 415, 416, 417, 418, 419, 420, 421, 422, 423, 424, 425, 426, 427, 428, 429, 430, 431, 432, 433, 434, 435, 436, 437 and 438.

4. A double-stranded ribonucleic acid molecule of claim 3, wherein the antisense strand further comprises a 3′ overhang of 1-5 nucleotides in length.

5. A double-stranded ribonucleic acid molecule of claim 4, wherein the overhang of the antisense strand comprises uracil or nucleotides which are at least 90% complementary to the mRNA encoding Factor VII.

6. A double-stranded ribonucleic acid molecule of claim 4, wherein the sense strand further comprises a 3′ overhang of 1-5 nucleotides in length.

7. A double-stranded ribonucleic acid molecule of claim 6, wherein the overhang of the sense strand comprises uracil or nucleotides which are at least 90% identical to the mRNA encoding Factor VII.

8. A double-stranded ribonucleic acid molecule of claim 1, wherein said sense strand is selected from the group consisting of the nucleic acid sequences depicted in SEQ ID Nos: 413, 415, 417, 419, 421, 423, 425, 427, 429, 431, 433, 435, and 437 and said antisense strand is selected from the group consisting of the nucleic acid sequences depicted in SEQ ID Nos: 414, 416, 418, 420, 422, 424, 426, 428, 430, 432, 434, 436 and 438, wherein said double-stranded ribonucleic acid molecule comprises the sequence pairs selected from the group consisting of SEQ ID NOs: 413/414, 415/416, 417/418, 419/420, 421/422, 423/424, 425/426, 427/428, 429/430, 431/432, 433/434, 435/436 and 437/438.

9. A double-stranded ribonucleic acid molecule of claim 1, wherein at least one strand of said double-stranded ribonucleic acid molecule has a half-life of at least 24 hours.

10. A double-stranded ribonucleic acid molecule of claim 1, wherein said double-stranded ribonucleic acid molecule is non-immunostimulatory.

11. A double-stranded ribonucleic acid molecule of claim 1, wherein said double-stranded ribonucleic acid molecule comprises at least one modified nucleotide.

12. A double-stranded ribonucleic acid molecule of claim 11, wherein said modified nucleotide is selected from the from the group consisting of a 2′-O-methyl modified nucleotide, a nucleotide comprising a 5′-phosphorothioate group, and a terminal nucleotide linked to a cholesteryl derivative or dodecanoic acid bisdecylamide group, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

13. A double-stranded ribonucleic acid molecule of claim 11, wherein said modified nucleotide is a 2′-O-methyl modified nucleotide, a nucleotide comprising a 5′-phosphorothioate group, and a deoxythymidine.

14. A double-stranded ribonucleic acid molecule of claim 1, wherein said sense strand is selected from the group consisting of the nucleic acid sequences depicted in SEQ ID Nos: 1, 3, 5, 7, 9, 11, 13, 15, 17, 19, 21, 23 and 25 and said antisense strand is selected from the group consisting of the nucleic acid sequences depicted in SEQ ID Nos: 2, 4, 6, 8, 10, 12, 14, 16, 18, 20, 22, 24 and 26, wherein said double-stranded ribonucleic acid molecule comprises the sequence pairs selected from the group consisting of SEQ ID NOs: 1/2, 3/4, 5/6, 7/8, 9/10, 11/12, 13/14, 15/16, 17/18, 19/20, 21/22, 23/24 and 25/26.

15. A nucleic acid sequence encoding a sense strand and/or an antisense strand comprised in the double-stranded ribonucleic acid molecule as defined in claim 1.

16. A vector comprising a regulatory sequence operably linked to a nucleotide sequence that encodes at least one of a sense strand or an antisense strand comprised in the double-stranded ribonucleic acid molecule as defined in claim 1 or comprising the nucleic acid sequence of claim 15.

17. A cell, tissue or non-human organism comprising the double-stranded ribonucleic acid molecule as defined in claim 1, the nucleic acid molecule of claim 15 or the vector of claim 16.

18. A pharmaceutical composition comprising the double-stranded ribonucleic acid molecule as defined in claim 1, the nucleic acid molecule of claim 15, the vector of claim 16 or the cell or tissue of claim 17.

19. A pharmaceutical composition of claim 18, further comprising a pharmaceutically acceptable carrier, stablilizer and/or diluent.

20. A method for inhibiting the expression of Factor VII gene in a cell, a tissue or an organism comprising the following steps:
(a) introducing into the cell, tissue or organism the double-stranded ribonucleic acid molecule as defined in claim 1, the nucleic acid molecule of claim 15, or the vector of claim 16; and
(b) maintaining the cell, tissue or organism produced in step (a) for a time sufficient to obtain degradation of the mRNA transcript of a Factor VII gene, thereby inhibiting expression of a Factor VII gene in the cell.


SEQUENCE LISTING (761 sequences)

SEQ ID NO: 1 - DNA, 21 bp, Artificial Sequence
  Description of Artificial Sequence Synthetic oligonucleotide
uucugguucuuauccauuatt

SEQ ID NO: 2 - DNA, 21 bp, Artificial Sequence
  Description of Artificial Sequence Synthetic oligonucleotide
uaauggauaagaaccagaatt

SEQ ID NO: 3 - DNA, 21 bp, Artificial Sequence
  Description of Artificial Sequence Synthetic oligonucleotide
gacacagagauggaauagatt

SEQ ID NO: 4 - DNA, 21 bp, Artificial Sequence
  Description of Artificial Sequence Synthetic oligonucleotide
ucuauuccaucucuguguctt

SEQ ID NO: 5 - DNA, 21 bp, Artificial Sequence
  Description of Artificial Sequence Synthetic oligonucleotide
gcaccaaaucccauauauutt

SEQ ID NO: 6 - DNA, 21 bp, Artificial Sequence
  Description of Artificial Sequence Synthetic oligonucleotide
aauauaugggauuuggugctt

SEQ ID NO: 7 - DNA, 21 bp, Artificial Sequence
  Description of Artificial Sequence Synthetic oligonucleotide
gaaaaauaccuauucuagatt

SEQ ID NO: 8 - DNA, 21 bp, Artificial Sequence
  Description of Artificial Sequence Synthetic oligonucleotide
ucuagaauagguauuuuuctt

SEQ ID NO: 9 - DNA, 21 bp, Artificial Sequence
  Description of Artificial Sequence Synthetic oligonucleotide
aaagccaaggcugcgucgatt

SEQ ID NO: 10 - DNA, 21 bp, Artificial Sequence
  Description of Artificial Sequence Synthetic oligonucleotide
ucgacgcagccuuggcuuutt

SEQ ID NO: 11 - DNA, 21 bp, Artificial Sequence
  Description of Artificial Sequence Synthetic oligonucleotide
gagauaugcacacaccgautt

SEQ ID NO: 12 - DNA, 21 bp, Artificial Sequence
  Description of Artificial Sequence Synthetic oligonucleotide
aucggugugugcauaucuctt

SEQ ID NO: 13 - DNA, 21 bp, Artificial Sequence
  Description of Artificial Sequence Synthetic oligonucleotide
ugcaaaagcucaugcgcuctt

SEQ ID NO: 14 - DNA, 21 bp, Artificial Sequence
  Description of Artificial Sequence Synthetic oligonucleotide
gagcgcaugagcuuuugcatt

SEQ ID NO: 15 - DNA, 21 bp, Artificial Sequence
  Description of Artificial Sequence Synthetic oligonucleotide
acacaucagugcacacggatt

SEQ ID NO: 16 - DNA, 21 bp, Artificial Sequence
  Description of Artificial Sequence Synthetic oligonucleotide
uccgugugcacugaugugutt

SEQ ID NO: 17 - DNA, 21 bp, Artificial Sequence
  Description of Artificial Sequence Synthetic oligonucleotide
cuucgugcgcuucucauugtt

SEQ ID NO: 18 - DNA, 21 bp, Artificial Sequence
  Description of Artificial Sequence Synthetic oligonucleotide
caaugagaagcgcacgaagtt

SEQ ID NO: 19 - DNA, 21 bp, Artificial Sequence
  Description of Artificial Sequence Synthetic oligonucleotide
agauaugcacacacacggatt

SEQ ID NO: 20 - DNA, 21 bp, Artificial Sequence
  Description of Artificial Sequence Synthetic oligonucleotide
uccgugugugugcauaucutt

SEQ ID NO: 21 - DNA, 21 bp, Artificial Sequence
  Description of Artificial Sequence Synthetic oligonucleotide
cgugcgcuucucauugguctt

SEQ ID NO: 22 - DNA, 21 bp, Artificial Sequence
  Description of Artificial Sequence Synthetic oligonucleotide
gaccaaugagaagcgcacgtt

SEQ ID NO: 23 - DNA, 21 bp, Artificial Sequence
  Description of Artificial Sequence Synthetic oligonucleotide
agcuucacaauaaacggcutt

SEQ ID NO: 24 - DNA, 21 bp, Artificial Sequence
  Description of Artificial Sequence Synthetic oligonucleotide
agccguuuauugugaagcutt

SEQ ID NO: 25 - DNA, 21 bp, Artificial Sequence
  Description of Artificial Sequence Synthetic oligonucleotide
cccagcuucacaauaaacgtt

SEQ ID NO: 26 - DNA, 21 bp, Artificial Sequence
  Description of Artificial Sequence Synthetic oligonucleotide
cguuuauugugaagcugggtt

SEQ ID NO: 27 - DNA, 21 bp, Artificial Sequence
  Description of Artificial Sequence Synthetic oligonucleotide
gacaguagaggcaugaacatt

SEQ ID NO: 28 - DNA, 21 bp, Artificial Sequence
  Description of Artificial Sequence Synthetic oligonucleotide
uguucaugccucuacuguctt

SEQ ID NO: 29 - DNA, 21 bp, Artificial Sequence
  Description of Artificial Sequence Synthetic oligonucleotide
agccaaggcugcgucgaactt

SEQ ID NO: 30 - DNA, 21 bp, Artificial Sequence
  Description of Artificial Sequence Synthetic oligonucleotide
guucgacgcagccuuggcutt

SEQ ID NO: 31 - DNA, 21 bp, Artificial Sequence
  Description of Artificial Sequence Synthetic oligonucleotide
gagucagggacacacgcautt

SEQ ID NO: 32 - DNA, 21 bp, Artificial Sequence
  Description of Artificial Sequence Synthetic oligonucleotide
augcgugugucccugacuctt

SEQ ID NO: 33 - DNA, 21 bp, Artificial Sequence
  Description of Artificial Sequence Synthetic oligonucleotide
ccaaauaucacggaguacatt

SEQ ID NO: 34 - DNA, 21 bp, Artificial Sequence
  Description of Artificial Sequence Synthetic oligonucleotide
uguacuccgugauauuuggtt

SEQ ID NO: 35 - DNA, 21 bp, Artificial Sequence
  Description of Artificial Sequence Synthetic oligonucleotide
cgaugcacacgcacauagatt

SEQ ID NO: 36 - DNA, 21 bp, Artificial Sequence
  Description of Artificial Sequence Synthetic oligonucleotide
ucuaugugcgugugcaucgtt

SEQ ID NO: 37 - DNA, 21 bp, Artificial Sequence
  Description of Artificial Sequence Synthetic oligonucleotide
ccaugcaugguggcgaaugtt

SEQ ID NO: 38 - DNA, 21 bp, Artificial Sequence
  Description of Artificial Sequence Synthetic oligonucleotide
cauucgccaccaugcauggtt

SEQ ID NO: 39 - DNA, 21 bp, Artificial Sequence
  Description of Artificial Sequence Synthetic oligonucleotide
gugugaacgagaacggcggtt

SEQ ID NO: 40 - DNA, 21 bp, Artificial Sequence
  Description of Artificial Sequence Synthetic oligonucleotide
ccgccguucucguucacactt

SEQ ID NO: 41 - DNA, 21 bp, Artificial Sequence
  Description of Artificial Sequence Synthetic oligonucleotide
cugcccgaacggacguucutt

SEQ ID NO: 42 - DNA, 21 bp, Artificial Sequence
  Description of Artificial Sequence Synthetic oligonucleotide
agaacguccguucgggcagtt

SEQ ID NO: 43 - DNA, 21 bp, Artificial Sequence
  Description of Artificial Sequence Synthetic oligonucleotide
cuggcaccaaaucccauautt

SEQ ID NO: 44 - DNA, 21 bp, Artificial Sequence
  Description of Artificial Sequence Synthetic oligonucleotide
auaugggauuuggugccagtt

SEQ ID NO: 45 - DNA, 21 bp, Artificial Sequence
  Description of Artificial Sequence Synthetic oligonucleotide
ggucacacagagauacgcatt

SEQ ID NO: 46 - DNA, 21 bp, Artificial Sequence
  Description of Artificial Sequence Synthetic oligonucleotide
ugcguaucucugugugacctt

SEQ ID NO: 47 - DNA, 21 bp, Artificial Sequence
  Description of Artificial Sequence Synthetic oligonucleotide
cggacguucucugagaggatt

SEQ ID NO: 48 - DNA, 21 bp, Artificial Sequence
  Description of Artificial Sequence Synthetic oligonucleotide
uccucucagagaacguccgtt

SEQ ID NO: 49 - DNA, 21 bp, Artificial Sequence
  Description of Artificial Sequence Synthetic oligonucleotide
ugugcgcacacacagauautt

SEQ ID NO: 50 - DNA, 21 bp, Artificial Sequence
  Description of Artificial Sequence Synthetic oligonucleotide
auaucugugugugcgcacatt

SEQ ID NO: 51 - DNA, 21 bp, Artificial Sequence
  Description of Artificial Sequence Synthetic oligonucleotide
gcgcacacacaccgauguatt

SEQ ID NO: 52 - DNA, 21 bp, Artificial Sequence
  Description of Artificial Sequence Synthetic oligonucleotide
uacaucggugugugugcgctt

SEQ ID NO: 53 - DNA, 21 bp, Artificial Sequence
  Description of Artificial Sequence Synthetic oligonucleotide
augugcgcacacacagauatt

SEQ ID NO: 54 - DNA, 21 bp, Artificial Sequence
  Description of Artificial Sequence Synthetic oligonucleotide
uaucugugugugcgcacautt

SEQ ID NO: 55 - DNA, 21 bp, Artificial Sequence
  Description of Artificial Sequence Synthetic oligonucleotide
gucacacagagauacgcaatt

SEQ ID NO: 56 - DNA, 21 bp, Artificial Sequence
  Description of Artificial Sequence Synthetic oligonucleotide
uugcguaucucugugugactt

SEQ ID NO: 57 - DNA, 21 bp, Artificial Sequence
  Description of Artificial Sequence Synthetic oligonucleotide
gccaaugcacgcacacauctt

SEQ ID NO: 58 - DNA, 21 bp, Artificial Sequence
  Description of Artificial Sequence Synthetic oligonucleotide
gaugugugcgugcauuggctt

SEQ ID NO: 59 - DNA, 21 bp, Artificial Sequence
  Description of Artificial Sequence Synthetic oligonucleotide
ugaucugugugaacgagaatt

SEQ ID NO: 60 - DNA, 21 bp, Artificial Sequence
  Description of Artificial Sequence Synthetic oligonucleotide
uucucguucacacagaucatt

SEQ ID NO: 61 - DNA, 21 bp, Artificial Sequence
  Description of Artificial Sequence Synthetic oligonucleotide
gcggcccacuguuucgacatt

SEQ ID NO: 62 - DNA, 21 bp, Artificial Sequence
  Description of Artificial Sequence Synthetic oligonucleotide
ugucgaaacagugggccgctt

SEQ ID NO: 63 - DNA, 21 bp, Artificial Sequence
  Description of Artificial Sequence Synthetic oligonucleotide
caaugcacgcacacaucagtt

SEQ ID NO: 64 - DNA, 21 bp, Artificial Sequence
  Description of Artificial Sequence Synthetic oligonucleotide
cugaugugugcgugcauugtt

SEQ ID NO: 65 - DNA, 21 bp, Artificial Sequence
  Description of Artificial Sequence Synthetic oligonucleotide
cacaccgaugugcgcacactt

SEQ ID NO: 66 - DNA, 21 bp, Artificial Sequence
  Description of Artificial Sequence Synthetic oligonucleotide
gugugcgcacaucggugugtt

SEQ ID NO: 67 - DNA, 21 bp, Artificial Sequence
  Description of Artificial Sequence Synthetic oligonucleotide
gcgguuguuuagcucucactt

SEQ ID NO: 68 - DNA, 21 bp, Artificial Sequence
  Description of Artificial Sequence Synthetic oligonucleotide
gugagagcuaaacaaccgctt

SEQ ID NO: 69 - DNA, 21 bp, Artificial Sequence
  Description of Artificial Sequence Synthetic oligonucleotide
accaugcaugguggcgaautt

SEQ ID NO: 70 - DNA, 21 bp, Artificial Sequence
  Description of Artificial Sequence Synthetic oligonucleotide
auucgccaccaugcauggutt

SEQ ID NO: 71 - DNA, 21 bp, Artificial Sequence
  Description of Artificial Sequence Synthetic oligonucleotide
acaucagugcacacggaugtt

SEQ ID NO: 72 - DNA, 21 bp, Artificial Sequence
  Description of Artificial Sequence Synthetic oligonucleotide
cauccgugugcacugaugutt

SEQ ID NO: 73 - DNA, 21 bp, Artificial Sequence
  Description of Artificial Sequence Synthetic oligonucleotide
ucccagcuucacaauaaactt

SEQ ID NO: 74 - DNA, 21 bp, Artificial Sequence
  Description of Artificial Sequence Synthetic oligonucleotide
guuuauugugaagcugggatt

SEQ ID NO: 75 - DNA, 21 bp, Artificial Sequence
  Description of Artificial Sequence Synthetic oligonucleotide
gagauuucaucauggucuctt

SEQ ID NO: 76 - DNA, 21 bp, Artificial Sequence
  Description of Artificial Sequence Synthetic oligonucleotide
gagaccaugaugaaaucuctt

SEQ ID NO: 77 - DNA, 21 bp, Artificial Sequence
  Description of Artificial Sequence Synthetic oligonucleotide
gaaggcgguuguuuagcuctt

SEQ ID NO: 78 - DNA, 21 bp, Artificial Sequence
  Description of Artificial Sequence Synthetic oligonucleotide
gagcuaaacaaccgccuuctt

SEQ ID NO: 79 - DNA, 21 bp, Artificial Sequence
  Description of Artificial Sequence Synthetic oligonucleotide
ccucugaaggcgguuguuutt

SEQ ID NO: 80 - DNA, 21 bp, Artificial Sequence
  Description of Artificial Sequence Synthetic oligonucleotide
aaacaaccgccuucagaggtt

SEQ ID NO: 81 - DNA, 21 bp, Artificial Sequence
  Description of Artificial Sequence Synthetic oligonucleotide
cugugugaacgagaacggctt

SEQ ID NO: 82 - DNA, 21 bp, Artificial Sequence
  Description of Artificial Sequence Synthetic oligonucleotide
gccguucucguucacacagtt

SEQ ID NO: 83 - DNA, 21 bp, Artificial Sequence
  Description of Artificial Sequence Synthetic oligonucleotide
ugcccgaacggacguucuctt

SEQ ID NO: 84 - DNA, 21 bp, Artificial Sequence
  Description of Artificial Sequence Synthetic oligonucleotide
gagaacguccguucgggcatt

SEQ ID NO: 85 - DNA, 21 bp, Artificial Sequence
  Description of Artificial Sequence Synthetic oligonucleotide
uggcaccaaaucccauauatt

SEQ ID NO: 86 - DNA, 21 bp, Artificial Sequence
  Description of Artificial Sequence Synthetic oligonucleotide
uauaugggauuuggugccatt

SEQ ID NO: 87 - DNA, 21 bp, Artificial Sequence
  Description of Artificial Sequence Synthetic oligonucleotide
auacgcaaacacaccgaugtt

SEQ ID NO: 88 - DNA, 21 bp, Artificial Sequence
  Description of Artificial Sequence Synthetic oligonucleotide
caucgguguguuugcguautt

SEQ ID NO: 89 - DNA, 21 bp, Artificial Sequence
  Description of Artificial Sequence Synthetic oligonucleotide
cuguccucugaaggcgguutt

SEQ ID NO: 90 - DNA, 21 bp, Artificial Sequence
  Description of Artificial Sequence Synthetic oligonucleotide
aaccgccuucagaggacagtt

SEQ ID NO: 91 - DNA, 21 bp, Artificial Sequence
  Description of Artificial Sequence Synthetic oligonucleotide
caccaagcgcuccugucggtt

SEQ ID NO: 92 - DNA, 21 bp, Artificial Sequence
  Description of Artificial Sequence Synthetic oligonucleotide
ccgacaggagcgcuuggugtt

SEQ ID NO: 93 - DNA, 21 bp, Artificial Sequence
  Description of Artificial Sequence Synthetic oligonucleotide
ccagcuucacaauaaacggtt

SEQ ID NO: 94 - DNA, 21 bp, Artificial Sequence
  Description of Artificial Sequence Synthetic oligonucleotide
ccguuuauugugaagcuggtt

SEQ ID NO: 95 - DNA, 21 bp, Artificial Sequence
  Description of Artificial Sequence Synthetic oligonucleotide
augccaaugcacgcacacatt

SEQ ID NO: 96 - DNA, 21 bp, Artificial Sequence
  Description of Artificial Sequence Synthetic oligonucleotide
ugugugcgugcauuggcautt

SEQ ID NO: 97 - DNA, 21 bp, Artificial Sequence
  Description of Artificial Sequence Synthetic oligonucleotide
cacacaucagugcacacggtt

SEQ ID NO: 98 - DNA, 21 bp, Artificial Sequence
  Description of Artificial Sequence Synthetic oligonucleotide
ccgugugcacugaugugugtt

SEQ ID NO: 99 - DNA, 21 bp, Artificial Sequence
  Description of Artificial Sequence Synthetic oligonucleotide
gucacggaaggugggagactt

SEQ ID NO: 100 - DNA, 21 bp, Artificial Sequence
  Description of Artificial Sequence Synthetic oligonucleotide
gucucccaccuuccgugactt

SEQ ID NO: 101 - DNA, 21 bp, Artificial Sequence
  Description of Artificial Sequence Synthetic oligonucleotide
acacagagauacgcaaacatt

SEQ ID NO: 102 - DNA, 21 bp, Artificial Sequence
  Description of Artificial Sequence Synthetic oligonucleotide
uguuugcguaucucugugutt

SEQ ID NO: 103 - DNA, 21 bp, Artificial Sequence
  Description of Artificial Sequence Synthetic oligonucleotide
acaugccaaugcacgcacatt

SEQ ID NO: 104 - DNA, 21 bp, Artificial Sequence
  Description of Artificial Sequence Synthetic oligonucleotide
ugugcgugcauuggcaugutt

SEQ ID NO: 105 - DNA, 21 bp, Artificial Sequence
  Description of Artificial Sequence Synthetic oligonucleotide
gcacguacgucccgggcactt

SEQ ID NO: 106 - DNA, 21 bp, Artificial Sequence
  Description of Artificial Sequence Synthetic oligonucleotide
gugcccgggacguacgugctt

SEQ ID NO: 107 - DNA, 21 bp, Artificial Sequence
  Description of Artificial Sequence Synthetic oligonucleotide
gggagugccaagguugucctt

SEQ ID NO: 108 - DNA, 21 bp, Artificial Sequence
  Description of Artificial Sequence Synthetic oligonucleotide
ggacaaccuuggcacuccctt

SEQ ID NO: 109 - DNA, 21 bp, Artificial Sequence
  Description of Artificial Sequence Synthetic oligonucleotide
uauacacauggaugcacgctt

SEQ ID NO: 110 - DNA, 21 bp, Artificial Sequence
  Description of Artificial Sequence Synthetic oligonucleotide
gcgugcauccauguguauatt

SEQ ID NO: 111 - DNA, 21 bp, Artificial Sequence
  Description of Artificial Sequence Synthetic oligonucleotide
guccucugaaggcgguugutt

SEQ ID NO: 112 - DNA, 21 bp, Artificial Sequence
  Description of Artificial Sequence Synthetic oligonucleotide
acaaccgccuucagaggactt

SEQ ID NO: 113 - DNA, 21 bp, Artificial Sequence
  Description of Artificial Sequence Synthetic oligonucleotide
gcccacuguuucgacaaaatt

SEQ ID NO: 114 - DNA, 21 bp, Artificial Sequence
  Description of Artificial Sequence Synthetic oligonucleotide
uuuugucgaaacagugggctt

SEQ ID NO: 115 - DNA, 21 bp, Artificial Sequence
  Description of Artificial Sequence Synthetic oligonucleotide
cacgcacauagagauaugctt

SEQ ID NO: 116 - DNA, 21 bp, Artificial Sequence
  Description of Artificial Sequence Synthetic oligonucleotide
gcauaucucuaugugcgugtt

SEQ ID NO: 117 - DNA, 21 bp, Artificial Sequence
  Description of Artificial Sequence Synthetic oligonucleotide
gccggcgcgccaacgcguutt

SEQ ID NO: 118 - DNA, 21 bp, Artificial Sequence
  Description of Artificial Sequence Synthetic oligonucleotide
aacgcguuggcgcgccggctt

SEQ ID NO: 119 - DNA, 21 bp, Artificial Sequence
  Description of Artificial Sequence Synthetic oligonucleotide
gcucagagaguggacucgatt

SEQ ID NO: 120 - DNA, 21 bp, Artificial Sequence
  Description of Artificial Sequence Synthetic oligonucleotide
ucgaguccacucucugagctt

SEQ ID NO: 121 - DNA, 21 bp, Artificial Sequence
  Description of Artificial Sequence Synthetic oligonucleotide
ccucagcgagcacgacgggtt

SEQ ID NO: 122 - DNA, 21 bp, Artificial Sequence
  Description of Artificial Sequence Synthetic oligonucleotide
cccgucgugcucgcugaggtt

SEQ ID NO: 123 - DNA, 21 bp, Artificial Sequence
  Description of Artificial Sequence Synthetic oligonucleotide
uucgugcgcuucucauuggtt

SEQ ID NO: 124 - DNA, 21 bp, Artificial Sequence
  Description of Artificial Sequence Synthetic oligonucleotide
ccaaugagaagcgcacgaatt

SEQ ID NO: 125 - DNA, 21 bp, Artificial Sequence
  Description of Artificial Sequence Synthetic oligonucleotide
gaaagccaaggcugcgucgtt

SEQ ID NO: 126 - DNA, 21 bp, Artificial Sequence
  Description of Artificial Sequence Synthetic oligonucleotide
cgacgcagccuuggcuuuctt

SEQ ID NO: 127 - DNA, 21 bp, Artificial Sequence
  Description of Artificial Sequence Synthetic oligonucleotide
gaccagcuccaguccuauatt

SEQ ID NO: 128 - DNA, 21 bp, Artificial Sequence
  Description of Artificial Sequence Synthetic oligonucleotide
uauaggacuggagcugguctt

SEQ ID NO: 129 - DNA, 21 bp, Artificial Sequence
  Description of Artificial Sequence Synthetic oligonucleotide
ugcgcacacacaccgaugutt

SEQ ID NO: 130 - DNA, 21 bp, Artificial Sequence
  Description of Artificial Sequence Synthetic oligonucleotide
acaucggugugugugcgcatt

SEQ ID NO: 131 - DNA, 21 bp, Artificial Sequence
  Description of Artificial Sequence Synthetic oligonucleotide
agagauuucaucauggucutt

SEQ ID NO: 132 - DNA, 21 bp, Artificial Sequence
  Description of Artificial Sequence Synthetic oligonucleotide
agaccaugaugaaaucucutt

SEQ ID NO: 133 - DNA, 21 bp, Artificial Sequence
  Description of Artificial Sequence Synthetic oligonucleotide
caaauaucacggaguacautt

SEQ ID NO: 134 - DNA, 21 bp, Artificial Sequence
  Description of Artificial Sequence Synthetic oligonucleotide
auguacuccgugauauuugtt

SEQ ID NO: 135 - DNA, 21 bp, Artificial Sequence
  Description of Artificial Sequence Synthetic oligonucleotide
acgcacacaucagugcacatt

SEQ ID NO: 136 - DNA, 21 bp, Artificial Sequence
  Description of Artificial Sequence Synthetic oligonucleotide
ugugcacugaugugugcgutt

SEQ ID NO: 137 - DNA, 21 bp, Artificial Sequence
  Description of Artificial Sequence Synthetic oligonucleotide
caccaccaaccacgacauctt

SEQ ID NO: 138 - DNA, 21 bp, Artificial Sequence
  Description of Artificial Sequence Synthetic oligonucleotide
gaugucgugguugguggugtt

SEQ ID NO: 139 - DNA, 21 bp, Artificial Sequence
  Description of Artificial Sequence Synthetic oligonucleotide
uggacucgaugccaucccutt

SEQ ID NO: 140 - DNA, 21 bp, Artificial Sequence
  Description of Artificial Sequence Synthetic oligonucleotide
agggauggcaucgaguccatt

SEQ ID NO: 141 - DNA, 21 bp, Artificial Sequence
  Description of Artificial Sequence Synthetic oligonucleotide
cucugccugcccgaacggatt

SEQ ID NO: 142 - DNA, 21 bp, Artificial Sequence
  Description of Artificial Sequence Synthetic oligonucleotide
uccguucgggcaggcagagtt

SEQ ID NO: 143 - DNA, 21 bp, Artificial Sequence
  Description of Artificial Sequence Synthetic oligonucleotide
uucugugccggcuacucggtt

SEQ ID NO: 144 - DNA, 21 bp, Artificial Sequence
  Description of Artificial Sequence Synthetic oligonucleotide
ccgaguagccggcacagaatt

SEQ ID NO: 145 - DNA, 21 bp, Artificial Sequence
  Description of Artificial Sequence Synthetic oligonucleotide
cacguacgucccgggcacctt

SEQ ID NO: 146 - DNA, 21 bp, Artificial Sequence
  Description of Artificial Sequence Synthetic oligonucleotide
ggugcccgggacguacgugtt

SEQ ID NO: 147 - DNA, 21 bp, Artificial Sequence
  Description of Artificial Sequence Synthetic oligonucleotide
ccucugccugcccgaacggtt

SEQ ID NO: 148 - DNA, 21 bp, Artificial Sequence
  Description of Artificial Sequence Synthetic oligonucleotide
ccguucgggcaggcagaggtt

SEQ ID NO: 149 - DNA, 21 bp, Artificial Sequence
  Description of Artificial Sequence Synthetic oligonucleotide
gcgcgccaacgcguuccugtt

SEQ ID NO: 150 - DNA, 21 bp, Artificial Sequence
  Description of Artificial Sequence Synthetic oligonucleotide
caggaacgcguuggcgcgctt

SEQ ID NO: 151 - DNA, 21 bp, Artificial Sequence
  Description of Artificial Sequence Synthetic oligonucleotide
ggcccacuguuucgacaaatt

SEQ ID NO: 152 - DNA, 21 bp, Artificial Sequence
  Description of Artificial Sequence Synthetic oligonucleotide
uuugucgaaacagugggcctt

SEQ ID NO: 153 - DNA, 21 bp, Artificial Sequence
  Description of Artificial Sequence Synthetic oligonucleotide
agaucuucaaggacgcggatt

SEQ ID NO: 154 - DNA, 21 bp, Artificial Sequence
  Description of Artificial Sequence Synthetic oligonucleotide
uccgcguccuugaagaucutt

SEQ ID NO: 155 - DNA, 21 bp, Artificial Sequence
  Description of Artificial Sequence Synthetic oligonucleotide
auguauuucucccuucgcutt

SEQ ID NO: 156 - DNA, 21 bp, Artificial Sequence
  Description of Artificial Sequence Synthetic oligonucleotide
agcgaagggagaaauacautt

SEQ ID NO: 157 - DNA, 21 bp, Artificial Sequence
  Description of Artificial Sequence Synthetic oligonucleotide
gauaugcacacaccgaugutt

SEQ ID NO: 158 - DNA, 21 bp, Artificial Sequence
  Description of Artificial Sequence Synthetic oligonucleotide
acaucggugugugcauauctt

SEQ ID NO: 159 - DNA, 21 bp, Artificial Sequence
  Description of Artificial Sequence Synthetic oligonucleotide
uacugcagugaccacacggtt

SEQ ID NO: 160 - DNA, 21 bp, Artificial Sequence
  Description of Artificial Sequence Synthetic oligonucleotide
ccguguggucacugcaguatt

SEQ ID NO: 161 - DNA, 21 bp, Artificial Sequence
  Description of Artificial Sequence Synthetic oligonucleotide
ccagggcugcgcaaccgugtt

SEQ ID NO: 162 - DNA, 21 bp, Artificial Sequence
  Description of Artificial Sequence Synthetic oligonucleotide
cacgguugcgcagcccuggtt

SEQ ID NO: 163 - DNA, 21 bp, Artificial Sequence
  Description of Artificial Sequence Synthetic oligonucleotide
caguccuauaucugcuucutt

SEQ ID NO: 164 - DNA, 21 bp, Artificial Sequence
  Description of Artificial Sequence Synthetic oligonucleotide
agaagcagauauaggacugtt

SEQ ID NO: 165 - DNA, 21 bp, Artificial Sequence
  Description of Artificial Sequence Synthetic oligonucleotide
ccugcccgaacggacguuctt

SEQ ID NO: 166 - DNA, 21 bp, Artificial Sequence
  Description of Artificial Sequence Synthetic oligonucleotide
gaacguccguucgggcaggtt

SEQ ID NO: 167 - DNA, 21 bp, Artificial Sequence
  Description of Artificial Sequence Synthetic oligonucleotide
cacgcaucacuaaaugcaatt

SEQ ID NO: 168 - DNA, 21 bp, Artificial Sequence
  Description of Artificial Sequence Synthetic oligonucleotide
uugcauuuagugaugcgugtt

SEQ ID NO: 169 - DNA, 21 bp, Artificial Sequence
  Description of Artificial Sequence Synthetic oligonucleotide
ugcacacaccgaugugcgctt

SEQ ID NO: 170 - DNA, 21 bp, Artificial Sequence
  Description of Artificial Sequence Synthetic oligonucleotide
gcgcacaucggugugugcatt

SEQ ID NO: 171 - DNA, 21 bp, Artificial Sequence
  Description of Artificial Sequence Synthetic oligonucleotide
cagcacguacgucccgggctt

SEQ ID NO: 172 - DNA, 21 bp, Artificial Sequence
  Description of Artificial Sequence Synthetic oligonucleotide
gcccgggacguacgugcugtt

SEQ ID NO: 173 - DNA, 21 bp, Artificial Sequence
  Description of Artificial Sequence Synthetic oligonucleotide
gugcgcuucucauuggucatt

SEQ ID NO: 174 - DNA, 21 bp, Artificial Sequence
  Description of Artificial Sequence Synthetic oligonucleotide
ugaccaaugagaagcgcactt

SEQ ID NO: 175 - DNA, 21 bp, Artificial Sequence
  Description of Artificial Sequence Synthetic oligonucleotide
aacggacguucucugagagtt

SEQ ID NO: 176 - DNA, 21 bp, Artificial Sequence
  Description of Artificial Sequence Synthetic oligonucleotide
cucucagagaacguccguutt

SEQ ID NO: 177 - DNA, 21 bp, Artificial Sequence
  Description of Artificial Sequence Synthetic oligonucleotide
gaucuucaaggacgcggagtt

SEQ ID NO: 178 - DNA, 21 bp, Artificial Sequence
  Description of Artificial Sequence Synthetic oligonucleotide
cuccgcguccuugaagauctt

SEQ ID NO: 179 - DNA, 21 bp, Artificial Sequence
  Description of Artificial Sequence Synthetic oligonucleotide
ccauggcagguccuguugutt

SEQ ID NO: 180 - DNA, 21 bp, Artificial Sequence
  Description of Artificial Sequence Synthetic oligonucleotide
acaacaggaccugccauggtt

SEQ ID NO: 181 - DNA, 21 bp, Artificial Sequence
  Description of Artificial Sequence Synthetic oligonucleotide
cuaugaacuacagccguggtt

SEQ ID NO: 182 - DNA, 21 bp, Artificial Sequence
  Description of Artificial Sequence Synthetic oligonucleotide
ccacggcuguaguucauagtt

SEQ ID NO: 183 - DNA, 21 bp, Artificial Sequence
  Description of Artificial Sequence Synthetic oligonucleotide
uacgcaaacacaccgaugctt

SEQ ID NO: 184 - DNA, 21 bp, Artificial Sequence
  Description of Artificial Sequence Synthetic oligonucleotide
gcaucgguguguuugcguatt

SEQ ID NO: 185 - DNA, 21 bp, Artificial Sequence
  Description of Artificial Sequence Synthetic oligonucleotide
caaggcugcgucgaacugutt

SEQ ID NO: 186 - DNA, 21 bp, Artificial Sequence
  Description of Artificial Sequence Synthetic oligonucleotide
acaguucgacgcagccuugtt

SEQ ID NO: 187 - DNA, 21 bp, Artificial Sequence
  Description of Artificial Sequence Synthetic oligonucleotide
agauaugcacacaccgaugtt

SEQ ID NO: 188 - DNA, 21 bp, Artificial Sequence
  Description of Artificial Sequence Synthetic oligonucleotide
caucggugugugcauaucutt

SEQ ID NO: 189 - DNA, 21 bp, Artificial Sequence
  Description of Artificial Sequence Synthetic oligonucleotide
cugcgucgaacuguccuggtt

SEQ ID NO: 190 - DNA, 21 bp, Artificial Sequence
  Description of Artificial Sequence Synthetic oligonucleotide
ccaggacaguucgacgcagtt

SEQ ID NO: 191 - DNA, 21 bp, Artificial Sequence
  Description of Artificial Sequence Synthetic oligonucleotide
augcgcacacacaccgaugtt

SEQ ID NO: 192 - DNA, 21 bp, Artificial Sequence
  Description of Artificial Sequence Synthetic oligonucleotide
caucggugugugugcgcautt

SEQ ID NO: 193 - DNA, 21 bp, Artificial Sequence
  Description of Artificial Sequence Synthetic oligonucleotide
ucugccugcccgaacggactt

SEQ ID NO: 194 - DNA, 21 bp, Artificial Sequence
  Description of Artificial Sequence Synthetic oligonucleotide
guccguucgggcaggcagatt

SEQ ID NO: 195 - DNA, 21 bp, Artificial Sequence
  Description of Artificial Sequence Synthetic oligonucleotide
gacuccggcaagcacggcutt

SEQ ID NO: 196 - DNA, 21 bp, Artificial Sequence
  Description of Artificial Sequence Synthetic oligonucleotide
agccgugcuugccggaguctt

SEQ ID NO: 197 - DNA, 21 bp, Artificial Sequence
  Description of Artificial Sequence Synthetic oligonucleotide
gacgcuggccuucgugcgctt

SEQ ID NO: 198 - DNA, 21 bp, Artificial Sequence
  Description of Artificial Sequence Synthetic oligonucleotide
gcgcacgaaggccagcguctt

SEQ ID NO: 199 - DNA, 21 bp, Artificial Sequence
  Description of Artificial Sequence Synthetic oligonucleotide
cgcacacacaccgauguactt

SEQ ID NO: 200 - DNA, 21 bp, Artificial Sequence
  Description of Artificial Sequence Synthetic oligonucleotide
guacaucggugugugugcgtt

SEQ ID NO: 201 - DNA, 21 bp, Artificial Sequence
  Description of Artificial Sequence Synthetic oligonucleotide
agauuucaucauggucucctt

SEQ ID NO: 202 - DNA, 21 bp, Artificial Sequence
  Description of Artificial Sequence Synthetic oligonucleotide
ggagaccaugaugaaaucutt

SEQ ID NO: 203 - DNA, 21 bp, Artificial Sequence
  Description of Artificial Sequence Synthetic oligonucleotide
aaggcugcgucgaacuguctt

SEQ ID NO: 204 - DNA, 21 bp, Artificial Sequence
  Description of Artificial Sequence Synthetic oligonucleotide
gacaguucgacgcagccuutt

SEQ ID NO: 205 - DNA, 21 bp, Artificial Sequence
  Description of Artificial Sequence Synthetic oligonucleotide
ugcgucuccuccgcacacctt

SEQ ID NO: 206 - DNA, 21 bp, Artificial Sequence
  Description of Artificial Sequence Synthetic oligonucleotide
ggugugcggaggagacgcatt

SEQ ID NO: 207 - DNA, 21 bp, Artificial Sequence
  Description of Artificial Sequence Synthetic oligonucleotide
aauaaacggcugcgucucctt

SEQ ID NO: 208 - DNA, 21 bp, Artificial Sequence
  Description of Artificial Sequence Synthetic oligonucleotide
ggagacgcagccguuuauutt

SEQ ID NO: 209 - DNA, 21 bp, Artificial Sequence
  Description of Artificial Sequence Synthetic oligonucleotide
auaugcacacacacggaugtt

SEQ ID NO: 210 - DNA, 21 bp, Artificial Sequence
  Description of Artificial Sequence Synthetic oligonucleotide
cauccgugugugugcauautt

SEQ ID NO: 211 - DNA, 21 bp, Artificial Sequence
  Description of Artificial Sequence Synthetic oligonucleotide
aaggcgguuguuuagcucutt

SEQ ID NO: 212 - DNA, 21 bp, Artificial Sequence
  Description of Artificial Sequence Synthetic oligonucleotide
agagcuaaacaaccgccuutt

SEQ ID NO: 213 - DNA, 21 bp, Artificial Sequence
  Description of Artificial Sequence Synthetic oligonucleotide
acgcaucacuaaaugcaagtt

SEQ ID NO: 214 - DNA, 21 bp, Artificial Sequence
  Description of Artificial Sequence Synthetic oligonucleotide
cuugcauuuagugaugcgutt

SEQ ID NO: 215 - DNA, 21 bp, Artificial Sequence
  Description of Artificial Sequence Synthetic oligonucleotide
cugccugcccgaacggacgtt

SEQ ID NO: 216 - DNA, 21 bp, Artificial Sequence
  Description of Artificial Sequence Synthetic oligonucleotide
cguccguucgggcaggcagtt

SEQ ID NO: 217 - DNA, 21 bp, Artificial Sequence
  Description of Artificial Sequence Synthetic oligonucleotide
cggcccacuguuucgacaatt

SEQ ID NO: 218 - DNA, 21 bp, Artificial Sequence
  Description of Artificial Sequence Synthetic oligonucleotide
uugucgaaacagugggccgtt

SEQ ID NO: 219 - DNA, 21 bp, Artificial Sequence
  Description of Artificial Sequence Synthetic oligonucleotide
cagggcugcgcaaccguggtt

SEQ ID NO: 220 - DNA, 21 bp, Artificial Sequence
  Description of Artificial Sequence Synthetic oligonucleotide
ccacgguugcgcagcccugtt

SEQ ID NO: 221 - DNA, 21 bp, Artificial Sequence
  Description of Artificial Sequence Synthetic oligonucleotide
uggucacacagagauacgctt

SEQ ID NO: 222 - DNA, 21 bp, Artificial Sequence
  Description of Artificial Sequence Synthetic oligonucleotide
gcguaucucugugugaccatt

SEQ ID NO: 223 - DNA, 21 bp, Artificial Sequence
  Description of Artificial Sequence Synthetic oligonucleotide
cuccugucggugccacgagtt

SEQ ID NO: 224 - DNA, 21 bp, Artificial Sequence
  Description of Artificial Sequence Synthetic oligonucleotide
cucguggcaccgacaggagtt

SEQ ID NO: 225 - DNA, 21 bp, Artificial Sequence
  Description of Artificial Sequence Synthetic oligonucleotide
caaggaccagcuccagucctt

SEQ ID NO: 226 - DNA, 21 bp, Artificial Sequence
  Description of Artificial Sequence Synthetic oligonucleotide
ggacuggagcugguccuugtt

SEQ ID NO: 227 - DNA, 21 bp, Artificial Sequence
  Description of Artificial Sequence Synthetic oligonucleotide
uucucauuggucagcggcutt

SEQ ID NO: 228 - DNA, 21 bp, Artificial Sequence
  Description of Artificial Sequence Synthetic oligonucleotide
agccgcugaccaaugagaatt

SEQ ID NO: 229 - DNA, 21 bp, Artificial Sequence
  Description of Artificial Sequence Synthetic oligonucleotide
gagaucuucaaggacgcggtt

SEQ ID NO: 230 - DNA, 21 bp, Artificial Sequence
  Description of Artificial Sequence Synthetic oligonucleotide
ccgcguccuugaagaucuctt

SEQ ID NO: 231 - DNA, 21 bp, Artificial Sequence
  Description of Artificial Sequence Synthetic oligonucleotide
agagaguggacucgaugcctt

SEQ ID NO: 232 - DNA, 21 bp, Artificial Sequence
  Description of Artificial Sequence Synthetic oligonucleotide
ggcaucgaguccacucucutt

SEQ ID NO: 233 - DNA, 21 bp, Artificial Sequence
  Description of Artificial Sequence Synthetic oligonucleotide
cucccaguacaucgaguggtt

SEQ ID NO: 234 - DNA, 21 bp, Artificial Sequence
  Description of Artificial Sequence Synthetic oligonucleotide
ccacucgauguacugggagtt

SEQ ID NO: 235 - DNA, 21 bp, Artificial Sequence
  Description of Artificial Sequence Synthetic oligonucleotide
agucagggacacacgcauctt

SEQ ID NO: 236 - DNA, 21 bp, Artificial Sequence
  Description of Artificial Sequence Synthetic oligonucleotide
gaugcgugugucccugacutt

SEQ ID NO: 237 - DNA, 21 bp, Artificial Sequence
  Description of Artificial Sequence Synthetic oligonucleotide
ccaucccugcagggccguctt

SEQ ID NO: 238 - DNA, 21 bp, Artificial Sequence
  Description of Artificial Sequence Synthetic oligonucleotide
gacggcccugcagggauggtt

SEQ ID NO: 239 - DNA, 21 bp, Artificial Sequence
  Description of Artificial Sequence Synthetic oligonucleotide
agucuucguaacccaggagtt

SEQ ID NO: 240 - DNA, 21 bp, Artificial Sequence
  Description of Artificial Sequence Synthetic oligonucleotide
cuccuggguuacgaagacutt

SEQ ID NO: 241 - DNA, 21 bp, Artificial Sequence
  Description of Artificial Sequence Synthetic oligonucleotide
caagcgcuccugucggugctt

SEQ ID NO: 242 - DNA, 21 bp, Artificial Sequence
  Description of Artificial Sequence Synthetic oligonucleotide
gcaccgacaggagcgcuugtt

SEQ ID NO: 243 - DNA, 21 bp, Artificial Sequence
  Description of Artificial Sequence Synthetic oligonucleotide
gguccucacugaccaugugtt

SEQ ID NO: 244 - DNA, 21 bp, Artificial Sequence
  Description of Artificial Sequence Synthetic oligonucleotide
cacauggucagugaggacctt

SEQ ID NO: 245 - DNA, 21 bp, Artificial Sequence
  Description of Artificial Sequence Synthetic oligonucleotide
aggcugcgucgaacugucctt

SEQ ID NO: 246 - DNA, 21 bp, Artificial Sequence
  Description of Artificial Sequence Synthetic oligonucleotide
ggacaguucgacgcagccutt

SEQ ID NO: 247 - DNA, 21 bp, Artificial Sequence
  Description of Artificial Sequence Synthetic oligonucleotide
ggacacacgcaucacuaaatt

SEQ ID NO: 248 - DNA, 21 bp, Artificial Sequence
  Description of Artificial Sequence Synthetic oligonucleotide
uuuagugaugcgugugucctt

SEQ ID NO: 249 - DNA, 21 bp, Artificial Sequence
  Description of Artificial Sequence Synthetic oligonucleotide
ugcacacacaccgaugcugtt

SEQ ID NO: 250 - DNA, 21 bp, Artificial Sequence
  Description of Artificial Sequence Synthetic oligonucleotide
cagcaucggugugugugcatt

SEQ ID NO: 251 - DNA, 21 bp, Artificial Sequence
  Description of Artificial Sequence Synthetic oligonucleotide
acugaaaugaacccucacatt

SEQ ID NO: 252 - DNA, 21 bp, Artificial Sequence
  Description of Artificial Sequence Synthetic oligonucleotide
ugugaggguucauuucagutt

SEQ ID NO: 253 - DNA, 21 bp, Artificial Sequence
  Description of Artificial Sequence Synthetic oligonucleotide
caguugaauauccauguggtt

SEQ ID NO: 254 - DNA, 21 bp, Artificial Sequence
  Description of Artificial Sequence Synthetic oligonucleotide
ccacauggauauucaacugtt

SEQ ID NO: 255 - DNA, 21 bp, Artificial Sequence
  Description of Artificial Sequence Synthetic oligonucleotide
ugagcaguacugcagugactt

SEQ ID NO: 256 - DNA, 21 bp, Artificial Sequence
  Description of Artificial Sequence Synthetic oligonucleotide
gucacugcaguacugcucatt

SEQ ID NO: 257 - DNA, 21 bp, Artificial Sequence
  Description of Artificial Sequence Synthetic oligonucleotide
gcugugagcaguacugcagtt

SEQ ID NO: 258 - DNA, 21 bp, Artificial Sequence
  Description of Artificial Sequence Synthetic oligonucleotide
cugcaguacugcucacagctt

SEQ ID NO: 259 - DNA, 21 bp, Artificial Sequence
  Description of Artificial Sequence Synthetic oligonucleotide
ggcugugagcaguacugcatt

SEQ ID NO: 260 - DNA, 21 bp, Artificial Sequence
  Description of Artificial Sequence Synthetic oligonucleotide
ugcaguacugcucacagcctt

SEQ ID NO: 261 - DNA, 21 bp, Artificial Sequence
  Description of Artificial Sequence Synthetic oligonucleotide
ggcugugagcaguacugcatt

SEQ ID NO: 262 - DNA, 21 bp, Artificial Sequence
  Description of Artificial Sequence Synthetic oligonucleotide
ugcaguacugcucacagcctt

SEQ ID NO: 263 - DNA, 21 bp, Artificial Sequence
  Description of Artificial Sequence Synthetic oligonucleotide
gugagcaguacugcagugatt

SEQ ID NO: 264 - DNA, 21 bp, Artificial Sequence
  Description of Artificial Sequence Synthetic oligonucleotide
ucacugcaguacugcucactt

SEQ ID NO: 265 - DNA, 21 bp, Artificial Sequence
  Description of Artificial Sequence Synthetic oligonucleotide
cccacaguugaauauccautt

SEQ ID NO: 266 - DNA, 21 bp, Artificial Sequence
  Description of Artificial Sequence Synthetic oligonucleotide
auggauauucaacugugggtt

SEQ ID NO: 267 - DNA, 21 bp, Artificial Sequence
  Description of Artificial Sequence Synthetic oligonucleotide
cugugagcaguacugcagutt

SEQ ID NO: 268 - DNA, 21 bp, Artificial Sequence
  Description of Artificial Sequence Synthetic oligonucleotide
acugcaguacugcucacagtt

SEQ ID NO: 269 - DNA, 21 bp, Artificial Sequence
  Description of Artificial Sequence Synthetic oligonucleotide
ccacaguugaauauccaugtt

SEQ ID NO: 270 - DNA, 21 bp, Artificial Sequence
  Description of Artificial Sequence Synthetic oligonucleotide
cauggauauucaacuguggtt

SEQ ID NO: 271 - DNA, 21 bp, Artificial Sequence
  Description of Artificial Sequence Synthetic oligonucleotide
acauguucugugccggcuatt

SEQ ID NO: 272 - DNA, 21 bp, Artificial Sequence
  Description of Artificial Sequence Synthetic oligonucleotide
uagccggcacagaacaugutt

SEQ ID NO: 273 - DNA, 21 bp, Artificial Sequence
  Description of Artificial Sequence Synthetic oligonucleotide
ucgaggaggcccgggagautt

SEQ ID NO: 274 - DNA, 21 bp, Artificial Sequence
  Description of Artificial Sequence Synthetic oligonucleotide
aucucccgggccuccucgatt

SEQ ID NO: 275 - DNA, 21 bp, Artificial Sequence
  Description of Artificial Sequence Synthetic oligonucleotide
gagcaguacugcagugacctt

SEQ ID NO: 276 - DNA, 21 bp, Artificial Sequence
  Description of Artificial Sequence Synthetic oligonucleotide
ggucacugcaguacugcuctt

SEQ ID NO: 277 - DNA, 21 bp, Artificial Sequence
  Description of Artificial Sequence Synthetic oligonucleotide
gcugugagcaguacugcagtt

SEQ ID NO: 278 - DNA, 21 bp, Artificial Sequence
  Description of Artificial Sequence Synthetic oligonucleotide
cugcaguacugcucacagctt

SEQ ID NO: 279 - DNA, 21 bp, Artificial Sequence
  Description of Artificial Sequence Synthetic oligonucleotide
cacaguugaauauccaugutt

SEQ ID NO: 280 - DNA, 21 bp, Artificial Sequence
  Description of Artificial Sequence Synthetic oligonucleotide
acauggauauucaacugugtt

SEQ ID NO: 281 - DNA, 21 bp, Artificial Sequence
  Description of Artificial Sequence Synthetic oligonucleotide
cauguucugugccggcuactt

SEQ ID NO: 282 - DNA, 21 bp, Artificial Sequence
  Description of Artificial Sequence Synthetic oligonucleotide
guagccggcacagaacaugtt

SEQ ID NO: 283 - DNA, 21 bp, Artificial Sequence
  Description of Artificial Sequence Synthetic oligonucleotide
cgaggaggcccgggagauctt

SEQ ID NO: 284 - DNA, 21 bp, Artificial Sequence
  Description of Artificial Sequence Synthetic oligonucleotide
gaucucccgggccuccucgtt

SEQ ID NO: 285 - DNA, 21 bp, Artificial Sequence
  Description of Artificial Sequence Synthetic oligonucleotide
cauguucugugccggcuactt

SEQ ID NO: 286 - DNA, 21 bp, Artificial Sequence
  Description of Artificial Sequence Synthetic oligonucleotide
guagccggcacagaacaugtt

SEQ ID NO: 287 - DNA, 21 bp, Artificial Sequence
  Description of Artificial Sequence Synthetic oligonucleotide
cccacaguugaauauccautt

SEQ ID NO: 288 - DNA, 21 bp, Artificial Sequence
  Description of Artificial Sequence Synthetic oligonucleotide
auggauauucaacugugggtt

SEQ ID NO: 289 - DNA, 21 bp, Artificial Sequence
  Description of Artificial Sequence Synthetic oligonucleotide
acauguucugugccggcuatt

SEQ ID NO: 290 - DNA, 21 bp, Artificial Sequence
  Description of Artificial Sequence Synthetic oligonucleotide
uagccggcacagaacaugutt

SEQ ID NO: 291 - DNA, 21 bp, Artificial Sequence
  Description of Artificial Sequence Synthetic oligonucleotide
ugugagcaguacugcagugtt

SEQ ID NO: 292 - DNA, 21 bp, Artificial Sequence
  Description of Artificial Sequence Synthetic oligonucleotide
cacugcaguacugcucacatt

SEQ ID NO: 293 - DNA, 21 bp, Artificial Sequence
  Description of Artificial Sequence Synthetic oligonucleotide
caguugaauauccauguggtt

SEQ ID NO: 294 - DNA, 21 bp, Artificial Sequence
  Description of Artificial Sequence Synthetic oligonucleotide
ccacauggauauucaacugtt

SEQ ID NO: 295 - DNA, 21 bp, Artificial Sequence
  Description of Artificial Sequence Synthetic oligonucleotide
ggccagcugcuggaccgugtt

SEQ ID NO: 296 - DNA, 21 bp, Artificial Sequence
  Description of Artificial Sequence Synthetic oligonucleotide
cacgguccagcagcuggcctt

SEQ ID NO: 297 - DNA, 21 bp, Artificial Sequence
  Description of Artificial Sequence Synthetic oligonucleotide
cacaguugaauauccaugutt

SEQ ID NO: 298 - DNA, 21 bp, Artificial Sequence
  Description of Artificial Sequence Synthetic oligonucleotide
acauggauauucaacugugtt

SEQ ID NO: 299 - DNA, 21 bp, Artificial Sequence
  Description of Artificial Sequence Synthetic oligonucleotide
gugagcaguacugcagugatt

SEQ ID NO: 300 - DNA, 21 bp, Artificial Sequence
  Description of Artificial Sequence Synthetic oligonucleotide
ucacugcaguacugcucactt

SEQ ID NO: 301 - DNA, 21 bp, Artificial Sequence
  Description of Artificial Sequence Synthetic oligonucleotide
auguucugugccggcuacutt

SEQ ID NO: 302 - DNA, 21 bp, Artificial Sequence
  Description of Artificial Sequence Synthetic oligonucleotide
aguagccggcacagaacautt

SEQ ID NO: 303 - DNA, 21 bp, Artificial Sequence
  Description of Artificial Sequence Synthetic oligonucleotide
ccacaguugaauauccaugtt

SEQ ID NO: 304 - DNA, 21 bp, Artificial Sequence
  Description of Artificial Sequence Synthetic oligonucleotide
cauggauauucaacuguggtt

SEQ ID NO: 305 - DNA, 21 bp, Artificial Sequence
  Description of Artificial Sequence Synthetic oligonucleotide
acaguugaauauccaugugtt

SEQ ID NO: 306 - DNA, 21 bp, Artificial Sequence
  Description of Artificial Sequence Synthetic oligonucleotide
cacauggauauucaacugutt

SEQ ID NO: 307 - DNA, 21 bp, Artificial Sequence
  Description of Artificial Sequence Synthetic oligonucleotide
auguucugugccggcuacutt

SEQ ID NO: 308 - DNA, 21 bp, Artificial Sequence
  Description of Artificial Sequence Synthetic oligonucleotide
aguagccggcacagaacautt

SEQ ID NO: 309 - DNA, 21 bp, Artificial Sequence
  Description of Artificial Sequence Synthetic oligonucleotide
cagcugcuggaccguggcgtt

SEQ ID NO: 310 - DNA, 21 bp, Artificial Sequence
  Description of Artificial Sequence Synthetic oligonucleotide
cgccacgguccagcagcugtt

SEQ ID NO: 311 - DNA, 21 bp, Artificial Sequence
  Description of Artificial Sequence Synthetic oligonucleotide
ggccagcugcuggaccgugtt

SEQ ID NO: 312 - DNA, 21 bp, Artificial Sequence
  Description of Artificial Sequence Synthetic oligonucleotide
cacgguccagcagcuggcctt

SEQ ID NO: 313 - DNA, 21 bp, Artificial Sequence
  Description of Artificial Sequence Synthetic oligonucleotide
ucgaggaggcccgggagautt

SEQ ID NO: 314 - DNA, 21 bp, Artificial Sequence
  Description of Artificial Sequence Synthetic oligonucleotide
aucucccgggccuccucgatt

SEQ ID NO: 315 - DNA, 21 bp, Artificial Sequence
  Description of Artificial Sequence Synthetic oligonucleotide
gccagcugcuggaccguggtt

SEQ ID NO: 316 - DNA, 21 bp, Artificial Sequence
  Description of Artificial Sequence Synthetic oligonucleotide
ccacgguccagcagcuggctt

SEQ ID NO: 317 - DNA, 21 bp, Artificial Sequence
  Description of Artificial Sequence Synthetic oligonucleotide
gggccagcugcuggaccgutt

SEQ ID NO: 318 - DNA, 21 bp, Artificial Sequence
  Description of Artificial Sequence Synthetic oligonucleotide
acgguccagcagcuggccctt

SEQ ID NO: 319 - DNA, 21 bp, Artificial Sequence
  Description of Artificial Sequence Synthetic oligonucleotide
uucgaggaggcccgggagatt

SEQ ID NO: 320 - DNA, 21 bp, Artificial Sequence
  Description of Artificial Sequence Synthetic oligonucleotide
ucucccgggccuccucgaatt

SEQ ID NO: 321 - DNA, 21 bp, Artificial Sequence
  Description of Artificial Sequence Synthetic oligonucleotide
agcugcuggaccguggcgctt

SEQ ID NO: 322 - DNA, 21 bp, Artificial Sequence
  Description of Artificial Sequence Synthetic oligonucleotide
gcgccacgguccagcagcutt

SEQ ID NO: 323 - DNA, 21 bp, Artificial Sequence
  Description of Artificial Sequence Synthetic oligonucleotide
agcugcuggaccguggcgctt

SEQ ID NO: 324 - DNA, 21 bp, Artificial Sequence
  Description of Artificial Sequence Synthetic oligonucleotide
gcgccacgguccagcagcutt

SEQ ID NO: 325 - DNA, 21 bp, Artificial Sequence
  Description of Artificial Sequence Synthetic oligonucleotide
cagcugcuggaccguggcgtt

SEQ ID NO: 326 - DNA, 21 bp, Artificial Sequence
  Description of Artificial Sequence Synthetic oligonucleotide
cgccacgguccagcagcugtt

SEQ ID NO: 327 - DNA, 21 bp, Artificial Sequence
  Description of Artificial Sequence Synthetic oligonucleotide
gggccagcugcuggaccgutt

SEQ ID NO: 328 - DNA, 21 bp, Artificial Sequence
  Description of Artificial Sequence Synthetic oligonucleotide
acgguccagcagcuggccctt

SEQ ID NO: 329 - DNA, 21 bp, Artificial Sequence
  Description of Artificial Sequence Synthetic oligonucleotide
uucgaggaggcccgggagatt

SEQ ID NO: 330 - DNA, 21 bp, Artificial Sequence
  Description of Artificial Sequence Synthetic oligonucleotide
ucucccgggccuccucgaatt

SEQ ID NO: 331 - DNA, 21 bp, Artificial Sequence
  Description of Artificial Sequence Synthetic oligonucleotide
cgaggaggcccgggagauctt

SEQ ID NO: 332 - DNA, 21 bp, Artificial Sequence
  Description of Artificial Sequence Synthetic oligonucleotide
gaucucccgggccuccucgtt

SEQ ID NO: 333 - DNA, 21 bp, Artificial Sequence
  Description of Artificial Sequence Synthetic oligonucleotide
ccagcugcuggaccguggctt

SEQ ID NO: 334 - DNA, 21 bp, Artificial Sequence
  Description of Artificial Sequence Synthetic oligonucleotide
gccacgguccagcagcuggtt

SEQ ID NO: 335 - DNA, 21 bp, Artificial Sequence
  Description of Artificial Sequence Synthetic oligonucleotide
gccagcugcuggaccguggtt

SEQ ID NO: 336 - DNA, 21 bp, Artificial Sequence
  Description of Artificial Sequence Synthetic oligonucleotide
ccacgguccagcagcuggctt

SEQ ID NO: 337 - DNA, 21 bp, Artificial Sequence
  Description of Artificial Sequence Synthetic oligonucleotide
ccagcugcuggaccguggctt

SEQ ID NO: 338 - DNA, 21 bp, Artificial Sequence
  Description of Artificial Sequence Synthetic oligonucleotide
gccacgguccagcagcuggtt

SEQ ID NO: 339 - DNA, 21 bp, Artificial Sequence
  Description of Artificial Sequence Synthetic oligonucleotide
cugcuggaccguggcgccatt

SEQ ID NO: 340 - DNA, 21 bp, Artificial Sequence
  Description of Artificial Sequence Synthetic oligonucleotide
uggcgccacgguccagcagtt

SEQ ID NO: 341 - DNA, 21 bp, Artificial Sequence
  Description of Artificial Sequence Synthetic oligonucleotide
acaguugaauauccaugugtt

SEQ ID NO: 342 - DNA, 21 bp, Artificial Sequence
  Description of Artificial Sequence Synthetic oligonucleotide
cacauggauauucaacugutt

SEQ ID NO: 343 - DNA, 21 bp, Artificial Sequence
  Description of Artificial Sequence Synthetic oligonucleotide
gcugcuggaccguggcgcctt

SEQ ID NO: 344 - DNA, 21 bp, Artificial Sequence
  Description of Artificial Sequence Synthetic oligonucleotide
ggcgccacgguccagcagctt

SEQ ID NO: 345 - DNA, 21 bp, Artificial Sequence
  Description of Artificial Sequence Synthetic oligonucleotide
gcugcuggaccguggcgcctt

SEQ ID NO: 346 - DNA, 21 bp, Artificial Sequence
  Description of Artificial Sequence Synthetic oligonucleotide
ggcgccacgguccagcagctt

SEQ ID NO: 347 - DNA, 21 bp, Artificial Sequence
  Description of Artificial Sequence Synthetic oligonucleotide
cugcuggaccguggcgccatt

SEQ ID NO: 348 - DNA, 21 bp, Artificial Sequence
  Description of Artificial Sequence Synthetic oligonucleotide
uggcgccacgguccagcagtt

SEQ ID NO: 349 - DNA, 35 bp, Artificial Sequence
  Description of Artificial Sequence Synthetic probe
tcgggcaggcagagggtttttgaagttaccgtttt

SEQ ID NO: 350 - DNA, 41 bp, Artificial Sequence
  Description of Artificial Sequence Synthetic probe
cgtcctctcagagaacgtccgttttttctgagtcaaagcat

SEQ ID NO: 351 - DNA, 38 bp, Artificial Sequence
  Description of Artificial Sequence Synthetic probe
aagcgcacgaaggccagtttttctcttggaaagaaagt

SEQ ID NO: 352 - DNA, 40 bp, Artificial Sequence
  Description of Artificial Sequence Synthetic probe
ccagccgctgaccaatgagtttttctcttggaaagaaagt

SEQ ID NO: 353 - DNA, 36 bp, Artificial Sequence
  Description of Artificial Sequence Synthetic probe
cggtccagcagctggcctttttgaagttaccgtttt

SEQ ID NO: 354 - DNA, 33 bp, Artificial Sequence
  Description of Artificial Sequence Synthetic probe
gggccgtggcgccatttttctgagtcaaagcat

SEQ ID NO: 355 - DNA, 42 bp, Artificial Sequence
  Description of Artificial Sequence Synthetic probe
cgttgaggaccatgagctccatttttctcttggaaagaaagt

SEQ ID NO: 356 - DNA, 17 bp, Artificial Sequence
  Description of Artificial Sequence Synthetic probe
ggtcatcagccggggca

SEQ ID NO: 357 - DNA, 20 bp, Artificial Sequence
  Description of Artificial Sequence Synthetic probe
gactgctgcaggcagtcctg

SEQ ID NO: 358 - DNA, 39 bp, Artificial Sequence
  Description of Artificial Sequence Synthetic probe
gggagtctcccaccttccgttttttgaagttaccgtttt

SEQ ID NO: 359 - DNA, 44 bp, Artificial Sequence
  Description of Artificial Sequence Synthetic probe
cagaacatgtactccgtgatatttgtttttctgagtcaaagcat

SEQ ID NO: 360 - DNA, 39 bp, Artificial Sequence
  Description of Artificial Sequence Synthetic probe
ccatccgagtagccggcatttttctcttggaaagaaagt

SEQ ID NO: 361 - DNA, 39 bp, Artificial Sequence
  Description of Artificial Sequence Synthetic probe
ccttgcaggagtccttgctgtttttgaagttaccgtttt

SEQ ID NO: 362 - DNA, 37 bp, Artificial Sequence
  Description of Artificial Sequence Synthetic probe
gtgggcctccactgtccctttttctgagtcaaagcat

SEQ ID NO: 363 - DNA, 39 bp, Artificial Sequence
  Description of Artificial Sequence Synthetic probe
cccggtagtgggtggcattttttctcttggaaagaaagt

SEQ ID NO: 364 - DNA, 38 bp, Artificial Sequence
  Description of Artificial Sequence Synthetic probe
cccgtcaggtaccacgtgctttttgaagttaccgtttt

SEQ ID NO: 365 - DNA, 37 bp, Artificial Sequence
  Description of Artificial Sequence Synthetic probe
tggccccagctgacgatgtttttctgagtcaaagcat

SEQ ID NO: 366 - DNA, 37 bp, Artificial Sequence
  Description of Artificial Sequence Synthetic probe
cacggttgcgcagccctttttctcttggaaagaaagt

SEQ ID NO: 367 - DNA, 39 bp, Artificial Sequence
  Description of Artificial Sequence Synthetic probe
gtgtacaccccaaagtggcctttttgaagttaccgtttt

SEQ ID NO: 368 - DNA, 40 bp, Artificial Sequence
  Description of Artificial Sequence Synthetic probe
tcgatgtactgggagaccctgtttttctgagtcaaagcat

SEQ ID NO: 369 - DNA, 41 bp, Artificial Sequence
  Description of Artificial Sequence Synthetic probe
gaatttgccatgggtggaattttttctcttggaaagaaagt

SEQ ID NO: 370 - DNA, 41 bp, Artificial Sequence
  Description of Artificial Sequence Synthetic probe
ggagggatctcgctcctggatttttctcttggaaagaaagt

SEQ ID NO: 371 - DNA, 40 bp, Artificial Sequence
  Description of Artificial Sequence Synthetic probe
ccccagccttctccatggttttttctcttggaaagaaagt

SEQ ID NO: 372 - DNA, 40 bp, Artificial Sequence
  Description of Artificial Sequence Synthetic probe
gctcccccctgcaaatgagtttttctcttggaaagaaagt

SEQ ID NO: 373 - DNA, 42 bp, Artificial Sequence
  Description of Artificial Sequence Synthetic probe
agccttgacggtgccatgtttttaggcataggacccgtgtct

SEQ ID NO: 374 - DNA, 45 bp, Artificial Sequence
  Description of Artificial Sequence Synthetic probe
gatgacaagcttcccgttctctttttaggcataggacccgtgtct

SEQ ID NO: 375 - DNA, 46 bp, Artificial Sequence
  Description of Artificial Sequence Synthetic probe
agatggtgatgggatttccatttttttaggcataggacccgtgtct

SEQ ID NO: 376 - DNA, 44 bp, Artificial Sequence
  Description of Artificial Sequence Synthetic probe
gcatcgccccacttgatttttttttaggcataggacccgtgtct

SEQ ID NO: 377 - DNA, 43 bp, Artificial Sequence
  Description of Artificial Sequence Synthetic probe
cacgacgtactcagcgccatttttaggcataggacccgtgtct

SEQ ID NO: 378 - DNA, 46 bp, Artificial Sequence
  Description of Artificial Sequence Synthetic probe
ggcagagatgatgacccttttgtttttaggcataggacccgtgtct

SEQ ID NO: 379 - DNA, 21 bp, Artificial Sequence
  Description of Artificial Sequence Synthetic probe
ggtgaagacgccagtggactc

SEQ ID NO: 380 - DNA, 41 bp, Artificial Sequence
  Description of Artificial Sequence Synthetic probe
ggttcctccatgcattccgttttttctcttggaaagaaagt

SEQ ID NO: 381 - DNA, 40 bp, Artificial Sequence
  Description of Artificial Sequence Synthetic probe
ggcctcctcgaatgtgcattttttctcttggaaagaaagt

SEQ ID NO: 382 - DNA, 39 bp, Artificial Sequence
  Description of Artificial Sequence Synthetic probe
ggcaggtgcctccgttcttttttctcttggaaagaaagt

SEQ ID NO: 383 - DNA, 40 bp, Artificial Sequence
  Description of Artificial Sequence Synthetic probe
ttcgggaggcagaagcagatttttctcttggaaagaaagt

SEQ ID NO: 384 - DNA, 39 bp, Artificial Sequence
  Description of Artificial Sequence Synthetic probe
cagttccggccgctgaagtttttctcttggaaagaaagt

SEQ ID NO: 385 - DNA, 42 bp, Artificial Sequence
  Description of Artificial Sequence Synthetic probe
agtgcgctcctgtttgtctcatttttctcttggaaagaaagt

SEQ ID NO: 386 - DNA, 44 bp, Artificial Sequence
  Description of Artificial Sequence Synthetic probe
ggtggtcctgaggatctccctttttaggcataggacccgtgtct

SEQ ID NO: 387 - DNA, 46 bp, Artificial Sequence
  Description of Artificial Sequence Synthetic probe
cccagaactggttcgtcttctctttttaggcataggacccgtgtct

SEQ ID NO: 388 - DNA, 50 bp, Artificial Sequence
  Description of Artificial Sequence Synthetic probe
caccattctcattgtcacagatcagctttttaggcataggacccgtgtct

SEQ ID NO: 389 - DNA, 42 bp, Artificial Sequence
  Description of Artificial Sequence Synthetic probe
gcgcgtgtctcccttgcgtttttaggcataggacccgtgtct

SEQ ID NO: 390 - DNA, 41 bp, Artificial Sequence
  Description of Artificial Sequence Synthetic probe
gcgtggcaccggcagattttttaggcataggacccgtgtct

SEQ ID NO: 391 - DNA, 22 bp, Artificial Sequence
  Description of Artificial Sequence Synthetic probe
tggtccccgtcagtatatgaag

SEQ ID NO: 392 - DNA, 20 bp, Artificial Sequence
  Description of Artificial Sequence Synthetic probe
ggcaagggtttgaggcacac

SEQ ID NO: 393 - DNA, 21 bp, Artificial Sequence
  Description of Artificial Sequence Synthetic probe
tgtacagccggaagtcgtctt

SEQ ID NO: 394 - DNA, 23 bp, Artificial Sequence
  Description of Artificial Sequence Synthetic probe
gtcactgcagtactgctcacagc

SEQ ID NO: 395 - DNA, 41 bp, Artificial Sequence
  Description of Artificial Sequence Synthetic probe
ccagcttcccattctcagcctttttctcttggaaagaaagt

SEQ ID NO: 396 - DNA, 41 bp, Artificial Sequence
  Description of Artificial Sequence Synthetic probe
tctcgctcctggaagatggttttttctcttggaaagaaagt

SEQ ID NO: 397 - DNA, 41 bp, Artificial Sequence
  Description of Artificial Sequence Synthetic probe
cccatttgatgttagcgggatttttctcttggaaagaaagt

SEQ ID NO: 398 - DNA, 42 bp, Artificial Sequence
  Description of Artificial Sequence Synthetic probe
cggagatgatgacccttttggtttttctcttggaaagaaagt

SEQ ID NO: 399 - DNA, 44 bp, Artificial Sequence
  Description of Artificial Sequence Synthetic probe
gatgggtttcccgttgatgatttttaggcataggacccgtgtct

SEQ ID NO: 400 - DNA, 47 bp, Artificial Sequence
  Description of Artificial Sequence Synthetic probe
gacatactcagcaccagcatcactttttaggcataggacccgtgtct

SEQ ID NO: 401 - DNA, 44 bp, Artificial Sequence
  Description of Artificial Sequence Synthetic probe
cccagccttctccatggtggtttttaggcataggacccgtgtct

SEQ ID NO: 402 - DNA, 21 bp, Artificial Sequence
  Description of Artificial Sequence Synthetic probe
ttgactgtgccgttgaacttg

SEQ ID NO: 403 - DNA, 22 bp, Artificial Sequence
  Description of Artificial Sequence Synthetic probe
tgaagacgccagtagactccac

SEQ ID NO: 404 - DNA, 19 bp, Artificial Sequence
  Description of Artificial Sequence Synthetic probe
ccccacccttcaggtgagc

SEQ ID NO: 405 - DNA, 17 bp, Artificial Sequence
  Description of Artificial Sequence Synthetic probe
ggcatcagcggaagggg

SEQ ID NO: 406 - DNA, 3075 bp, Homo sapiens
agtcccatggggaatgtcaacaggcaggggcagcactgcagagatttcatcatggtctcccaggccctcaggctcctctgccttctgcttgggcttcagggctgcctggctgcagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctagcccagcagccctggcctgtggagagaaagccaaggctgcgtcgaactgtcctggcaccaaatcccatatattcttctgcagttaatggggtagaggagggcatgggagggagggagaggtggggagggagacagagacagaaacagagagagacagagacagagagagactgagggagagactctgaggacatggagagagactcaaagagactccaagattcaaagagactaatagagacacagagatggaatagaaaagatgagaggcagaggcagacaggcgctggacagaggggcaggggagtgccaaggttgtcctggaggcagacagcccagctgagcctccttacctcccttcagccaagcccacctgcacgtgatctgctggcctcaggctgctgctctgccttcattgctggagacagtagaggcatgaacacacatggatgcacacacacacacgccaatgcacacacacagagatatgcacacacacggatgcacacacagatggtcacacagagatacgcaaacacaccgatgcacacgcacatagagatatgcacacacagatgcacacacagatatacacatggatgcacgcacatgccaatgcacgcacacatcagtgcacacggatgcacagagatatgcacacaccgatgtgcgcacacacagatatgcacacacatggatgagcacacacacaccaatgcgcacacacaccgatgtacacacacagatgcacacacagatgcacacacaccgatgctgactccatgtgtgctgtcctctgaaggcggttgtttagctctcacttttctggttcttatccattatcatcttcacttcagacaattcagaagcatcaccatgcatggtggcgaatgcccccaaactctcccccaaatgtatttctcccttcgctgggtgccgggctgcacagactattccccacctgcttcccagcttcacaataaacggctgcgtctcctccgcacacctgtggtgcctgccacccactgggttgcccatgattcatttttggagcccccggtgctcatcctctgagatgctcttttctttcacaattttcaacatcactgaaatgaaccctcacatggaagctattttttaaaaacaaaagctgtttgatagatgtttgaggctgtagctcccaggatcctgtggaattggatgttctctccctgccacagcccttgtcaatgatatttcacagagaccctgggagcacctgctcaagagtcagggacacacgcatcactaaatgcaagttcccaggccctggctgcagtgggaggacctggcaagctgcactcttgctgagtccccagggtggtggaagaagaatgagaaacacatgaacagagaaatggggaggtgacaaacagtgcccccactcagactccggcaagcacggctcagagagtggactcgatgccatccctgcagggccgtcctgggcaccactggcactcacagcagcaaggtgggcaccattggcactcacagcagcaaggcaggcaccagcaacccacctcgggggcactcaggcatcatctacttcagagcagacagggtctatgaactacagccgtgggctgcttccaaggcaccctgctcttgtaaataaagttttatgggaacacaaaaaaaaaaaaaaaaa

SEQ ID NO: 407 - DNA, 3141 bp, Homo sapiens
agtcccatggggaatgtcaacaggcaggggcagcactgcagagatttcatcatggtctcccaggccctcaggctcctctgccttctgcttgggcttcagggctgcctggctgcaggcggggtcgctaaggcctcaggaggagaaacacgggacatgccgtggaagccggggcctcacagagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctagcccagcagccctggcctgtggagagaaagccaaggctgcgtcgaactgtcctggcaccaaatcccatatattcttctgcagttaatggggtagaggagggcatgggagggagggagaggtggggagggagacagagacagaaacagagagagacagagacagagagagactgagggagagactctgaggacatggagagagactcaaagagactccaagattcaaagagactaatagagacacagagatggaatagaaaagatgagaggcagaggcagacaggcgctggacagaggggcaggggagtgccaaggttgtcctggaggcagacagcccagctgagcctccttacctcccttcagccaagcccacctgcacgtgatctgctggcctcaggctgctgctctgccttcattgctggagacagtagaggcatgaacacacatggatgcacacacacacacgccaatgcacacacacagagatatgcacacacacggatgcacacacagatggtcacacagagatacgcaaacacaccgatgcacacgcacatagagatatgcacacacagatgcacacacagatatacacatggatgcacgcacatgccaatgcacgcacacatcagtgcacacggatgcacagagatatgcacacaccgatgtgcgcacacacagatatgcacacacatggatgagcacacacacaccaatgcgcacacacaccgatgtacacacacagatgcacacacagatgcacacacaccgatgctgactccatgtgtgctgtcctctgaaggcggttgtttagctctcacttttctggttcttatccattatcatcttcacttcagacaattcagaagcatcaccatgcatggtggcgaatgcccccaaactctcccccaaatgtatttctcccttcgctgggtgccgggctgcacagactattccccacctgcttcccagcttcacaataaacggctgcgtctcctccgcacacctgtggtgcctgccacccactgggttgcccatgattcatttttggagcccccggtgctcatcctctgagatgctcttttctttcacaattttcaacatcactgaaatgaaccctcacatggaagctattttttaaaaacaaaagctgtttgatagatgtttgaggctgtagctcccaggatcctgtggaattggatgttctctccctgccacagcccttgtcaatgatatttcacagagaccctgggagcacctgctcaagagtcagggacacacgcatcactaaatgcaagttcccaggccctggctgcagtgggaggacctggcaagctgcactcttgctgagtccccagggtggtggaagaagaatgagaaacacatgaacagagaaatggggaggtgacaaacagtgcccccactcagactccggcaagcacggctcagagagtggactcgatgccatccctgcagggccgtcctgggcaccactggcactcacagcagcaaggtgggcaccattggcactcacagcagcaaggcaggcaccagcaacccacctcgggggcactcaggcatcatctacttcagagcagacagggtctatgaactacagccgtgggctgcttccaaggcaccctgctcttgtaaataaagttttatgggaacacaaaaaaaaaaaaaaaaa

SEQ ID NO: 408 - DNA, 751 bp, Macaca fascicularis
tttccccccgggactaagttccataaaccgcgatgggggtgcagctctaaaatcgtgaagttgggaagccggtggggatcggcctatgcagcccggcacatttgggggagagtttggggcatttgccaacatgcatggtgatgcttctgaattgtctgaaatgaagatgataatggataagaacgaggaaagtgagagctaaacaaccgcctttggaggacagcacacatggagtcagcatcggtgtgtgtgcatccgtgtgtgcatatctgtgtgtgtacattggtgtgtgtgcactcatccatgtgtgtgcatatctgtgtgcatctgtgtgtgtgcatatctgtgtatgtgcacatcggtgtgtgcatatctctctgcatccgtgtgggtgcattagtgtgtgtgtgcattggtgtggtgtgcacgcatccgtgtgtgcgcatatctctgtatgtgtgtgtgtcagtgtgtttgcatatctctgtgtgaccctctgtgtgtgcatccgtgtgtgtgcatatctctgtgtgtgtgcattggtgtgtgtgtatgcatccacgtgtgtacatgcctctagtgtctccagcaatgaaggcagaagaatcgatgggattttgtgccagaacagttctacacagccttggctttctctccaggggcagggctgctagctagggaaatggggctcgcagaagacgcctgggcgtggctctgagtgcatgagcttttgcagccactcgatgtactt

SEQ ID NO: 409 - DNA, 1508 bp, Artificial Sequence
  Description of Artificial Sequence Synthetic consensus sequence
atgctcgagcggccgccagtgtgatggatatctgcagaattcgcccttggaatgtcaacaggcagaggcagcgctgcagagatttcatcatggtctctcgagccctcgggctcctctgccttctgcttgggcttcagggctgtctggctgcagccaccttcctgccccaggcggggtcgctgaggcctcaggaggagaaaacacaggacctgctgtggaagccagggcctcacagagtcttcgtaacccaggaggaagcccatggcgtcctgcacaggcagcggcgcgccaactcgttcctggaggagctgcggccgggctccctggagagggagtgcaaggaggagcaatgctccttcgaggaggcccgggagatcttcaaggacctggagaggacgaagctgttctggatttcttacagtgatggggaccagtgtgcctcaaatccgtgccagaatgggggctcctgcaaggaccagctccagtcctatatctgcttctgcctcccttccttcgagggccggaactgtgagaagaacaaggacgaccagctgatctgcgtgaacgagaacggcggctgtgagcagtactgcagtgaccacgcgggtgccaagcgctcctgttggtgccacgaggggtactcgctgctggcagacggggtgtcctgcatgcccacagttgaatatccatgtggaaaaatacctattctggaaaaaagaaatgccagcaaaccccaaggccgaattgtcgggggcagggtgtgccccaaaggggagtgtccatggcaggtcctgttgttggtgaatggagctcagctgtgtggagggaccctgataaacaccatctgggtggtctctgcggcccactgtttcgacaaaatcaagagctggaggaacttgaccgcggtgctgggcgagcacgacctcagcgagcacgaaggggatgagcagagccggcgggtggcgcaggtcatcatccccagcacgtatgtcctgggcgccaccaaccacgacatcgcgctgctccgcctgcagcagcccgtggtcctcactgaccatgtggtgcccctctgcctgcccgaacggacgttctccgagaggacgctggccttcgtgcgcttctcgttggtcagcggctggggtcagctgctggaccgtggtgccacagccctggagctcatggccctcaacgtgccccggctgatgacccaggactgcctgcagcagtcacggaaggcggaagcctccccgaatatcacggagtacatgttctgtgccggctactcggacggcagcagggactcctgcaagggggacagtggaggcccacacgccacccgctaccggggcacgtggtacctgacaggcatcgtcagctggggccagggctgcgcggccgtgggccacttcggggtgtacaccagggtctcccagtacatcgagtggctgcaaaagctcatgcactcagagccacgcccaggcgtcctcctgcgagccccatttccctag

SEQ ID NO: 410 - DNA, 1242 bp, Cavia porcellus
ggcgtgctgcgcaggcagaggcgtgccaactcctttctggaggagctgtggccaggctccttggagacggaatgcatggaggaaccatgcacattcgaggaggcccgggagatcctcaggaccaccgagaagacgaaccagttctgggcttcatatactgacggggaccagtgtgcctcaaacccttgccagaacggaggcacctgccaagacgacttccggctgtacatctgcttctgcctcccgaacttcagcggccggaactgtgagacaaacaggagcgcactgctgatctgtgacaatgagaatggtggctgtgagcagtactgcagtgaccgcaagggagacacgcgcatctgccggtgccacgcggggtacactctgcagccggaccaagtatcctgcatgcccacagttgaatatccatgtgggaaaataccagttctggaaaaaaggaaggacagcaacatccaaggccgaattgtgggaggccagaagtgtcccagaggggagtgtccatggcaggccatcctgatgttcaatggggcaccactgtgtgggggctccctgctggagactggctgggtggtgtctgctgcccactgcttcgacaagcttcagagcttgaggaacctgtctgtggtgctgggtgagcatgacctcagcgaggttgatggcactgagcaggtgcgaaaggtgacacaagtcatcttcccggacacatatgtccggggccagaaagaccacgacatcgtgctcttgcggctgcagcggcccgtaaacctcacggactatgtggtgcctctctgcctacccgagacggccttcgcacagggcacactggccaaaatccgcttctcccgtgtcagtggttggggccagctgctggaccgtggcgccatggctgtggagctcatggccattgaggtgccacggctgatgaaccaggactgcctggaacagactcagaggacaaacagctcgccagtgatcaccaaaaacatgttctgtgccggctacttggacggcaccaaggacgcctgcaaaggggacagtgggggcccacacgccacgcagttccgtggcacgtggtacctgacaggtgtggtgagctggggtgaaggctgtgcggccgtgggccagctgggcgtctacaccagggtggcccgctacatcgagtggctgcgcaggctcatgcgctcccagccggggcgcagtgtcctgcgggccccgctcctctag

SEQ ID NO: 411 - DNA, 21 bp, Artificial Sequence
  Description of Artificial Sequence Synthetic oligonucleotide
cuuacgcugaguacuucgatt

SEQ ID NO: 412 - DNA, 21 bp, Artificial Sequence
  Description of Artificial Sequence Synthetic oligonucleotide
ucgaaguacucagcguaagtt

SEQ ID NO: 413 - DNA, 21 bp, Artificial Sequence
  Description of Artificial Sequence Synthetic oligonucleotide
uucugguucuuauccauuatt

SEQ ID NO: 414 - DNA, 21 bp, Artificial Sequence
  Description of Artificial Sequence Synthetic oligonucleotide
uaauggauaagaaccagaatt

SEQ ID NO: 415 - DNA, 21 bp, Artificial Sequence
  Description of Artificial Sequence Synthetic oligonucleotide
gacacagagauggaauagatt

SEQ ID NO: 416 - DNA, 21 bp, Artificial Sequence
  Description of Artificial Sequence Synthetic oligonucleotide
ucuauuccaucucuguguctt

SEQ ID NO: 417 - DNA, 21 bp, Artificial Sequence
  Description of Artificial Sequence Synthetic oligonucleotide
gcaccaaaucccauauauutt

SEQ ID NO: 418 - DNA, 21 bp, Artificial Sequence
  Description of Artificial Sequence Synthetic oligonucleotide
aauauaugggauuuggugctt

SEQ ID NO: 419 - DNA, 21 bp, Artificial Sequence
  Description of Artificial Sequence Synthetic oligonucleotide
gaaaaauaccuauucuagatt

SEQ ID NO: 420 - DNA, 21 bp, Artificial Sequence
  Description of Artificial Sequence Synthetic oligonucleotide
ucuagaauagguauuuuuctt

SEQ ID NO: 421 - DNA, 21 bp, Artificial Sequence
  Description of Artificial Sequence Synthetic oligonucleotide
aaagccaaggcugcgucgatt

SEQ ID NO: 422 - DNA, 21 bp, Artificial Sequence
  Description of Artificial Sequence Synthetic oligonucleotide
ucgacgcagccuuggcuuutt

SEQ ID NO: 423 - DNA, 21 bp, Artificial Sequence
  Description of Artificial Sequence Synthetic oligonucleotide
gagauaugcacacaccgautt

SEQ ID NO: 424 - DNA, 21 bp, Artificial Sequence
  Description of Artificial Sequence Synthetic oligonucleotide
aucggugugugcauaucuctt

SEQ ID NO: 425 - DNA, 21 bp, Artificial Sequence
  Description of Artificial Sequence Synthetic oligonucleotide
ugcaaaagcucaugcgcuctt

SEQ ID NO: 426 - DNA, 21 bp, Artificial Sequence
  Description of Artificial Sequence Synthetic oligonucleotide
gagcgcaugagcuuuugcatt

SEQ ID NO: 427 - DNA, 21 bp, Artificial Sequence
  Description of Artificial Sequence Synthetic oligonucleotide
acacaucagugcacacggatt

SEQ ID NO: 428 - DNA, 21 bp, Artificial Sequence
  Description of Artificial Sequence Synthetic oligonucleotide
uccgugugcacugaugugutt

SEQ ID NO: 429 - DNA, 21 bp, Artificial Sequence
  Description of Artificial Sequence Synthetic oligonucleotide
cuucgugcgcuucucauugtt

SEQ ID NO: 430 - DNA, 21 bp, Artificial Sequence
  Description of Artificial Sequence Synthetic oligonucleotide
caaugagaagcgcacgaagtt

SEQ ID NO: 431 - DNA, 21 bp, Artificial Sequence
  Description of Artificial Sequence Synthetic oligonucleotide
agauaugcacacacacggatt

SEQ ID NO: 432 - DNA, 21 bp, Artificial Sequence
  Description of Artificial Sequence Synthetic oligonucleotide
uccgugugugugcauaucutt

SEQ ID NO: 433 - DNA, 21 bp, Artificial Sequence
  Description of Artificial Sequence Synthetic oligonucleotide
cgugcgcuucucauugguctt

SEQ ID NO: 434 - DNA, 21 bp, Artificial Sequence
  Description of Artificial Sequence Synthetic oligonucleotide
gaccaaugagaagcgcacgtt

SEQ ID NO: 435 - DNA, 21 bp, Artificial Sequence
  Description of Artificial Sequence Synthetic oligonucleotide
agcuucacaauaaacggcutt

SEQ ID NO: 436 - DNA, 21 bp, Artificial Sequence
  Description of Artificial Sequence Synthetic oligonucleotide
agccguuuauugugaagcutt

SEQ ID NO: 437 - DNA, 21 bp, Artificial Sequence
  Description of Artificial Sequence Synthetic oligonucleotide
cccagcuucacaauaaacgtt

SEQ ID NO: 438 - DNA, 21 bp, Artificial Sequence
  Description of Artificial Sequence Synthetic oligonucleotide
cguuuauugugaagcugggtt

SEQ ID NO: 439 - DNA, 21 bp, Artificial Sequence
  Description of Artificial Sequence Synthetic oligonucleotide
gacaguagaggcaugaacatt

SEQ ID NO: 440 - DNA, 21 bp, Artificial Sequence
  Description of Artificial Sequence Synthetic oligonucleotide
uguucaugccucuacuguctt

SEQ ID NO: 441 - DNA, 21 bp, Artificial Sequence
  Description of Artificial Sequence Synthetic oligonucleotide
agccaaggcugcgucgaactt

SEQ ID NO: 442 - DNA, 21 bp, Artificial Sequence
  Description of Artificial Sequence Synthetic oligonucleotide
guucgacgcagccuuggcutt

SEQ ID NO: 443 - DNA, 21 bp, Artificial Sequence
  Description of Artificial Sequence Synthetic oligonucleotide
gagucagggacacacgcautt

SEQ ID NO: 444 - DNA, 21 bp, Artificial Sequence
  Description of Artificial Sequence Synthetic oligonucleotide
augcgugugucccugacuctt

SEQ ID NO: 445 - DNA, 21 bp, Artificial Sequence
  Description of Artificial Sequence Synthetic oligonucleotide
ccaaauaucacggaguacatt

SEQ ID NO: 446 - DNA, 21 bp, Artificial Sequence
  Description of Artificial Sequence Synthetic oligonucleotide
uguacuccgugauauuuggtt

SEQ ID NO: 447 - DNA, 21 bp, Artificial Sequence
  Description of Artificial Sequence Synthetic oligonucleotide
cgaugcacacgcacauagatt

SEQ ID NO: 448 - DNA, 21 bp, Artificial Sequence
  Description of Artificial Sequence Synthetic oligonucleotide
ucuaugugcgugugcaucgtt

SEQ ID NO: 449 - DNA, 21 bp, Artificial Sequence
  Description of Artificial Sequence Synthetic oligonucleotide
ccaugcaugguggcgaaugtt

SEQ ID NO: 450 - DNA, 21 bp, Artificial Sequence
  Description of Artificial Sequence Synthetic oligonucleotide
cauucgccaccaugcauggtt

SEQ ID NO: 451 - DNA, 21 bp, Artificial Sequence
  Description of Artificial Sequence Synthetic oligonucleotide
gugugaacgagaacggcggtt

SEQ ID NO: 452 - DNA, 21 bp, Artificial Sequence
  Description of Artificial Sequence Synthetic oligonucleotide
ccgccguucucguucacactt

SEQ ID NO: 453 - DNA, 21 bp, Artificial Sequence
  Description of Artificial Sequence Synthetic oligonucleotide
cugcccgaacggacguucutt

SEQ ID NO: 454 - DNA, 21 bp, Artificial Sequence
  Description of Artificial Sequence Synthetic oligonucleotide
agaacguccguucgggcagtt

SEQ ID NO: 455 - DNA, 21 bp, Artificial Sequence
  Description of Artificial Sequence Synthetic oligonucleotide
cuggcaccaaaucccauautt

SEQ ID NO: 456 - DNA, 21 bp, Artificial Sequence
  Description of Artificial Sequence Synthetic oligonucleotide
auaugggauuuggugccagtt

SEQ ID NO: 457 - DNA, 21 bp, Artificial Sequence
  Description of Artificial Sequence Synthetic oligonucleotide
ggucacacagagauacgcatt

SEQ ID NO: 458 - DNA, 21 bp, Artificial Sequence
  Description of Artificial Sequence Synthetic oligonucleotide
ugcguaucucugugugacctt

SEQ ID NO: 459 - DNA, 21 bp, Artificial Sequence
  Description of Artificial Sequence Synthetic oligonucleotide
cggacguucucugagaggatt

SEQ ID NO: 460 - DNA, 21 bp, Artificial Sequence
  Description of Artificial Sequence Synthetic oligonucleotide
uccucucagagaacguccgtt

SEQ ID NO: 461 - DNA, 21 bp, Artificial Sequence
  Description of Artificial Sequence Synthetic oligonucleotide
ugugcgcacacacagauautt

SEQ ID NO: 462 - DNA, 21 bp, Artificial Sequence
  Description of Artificial Sequence Synthetic oligonucleotide
auaucugugugugcgcacatt

SEQ ID NO: 463 - DNA, 21 bp, Artificial Sequence
  Description of Artificial Sequence Synthetic oligonucleotide
gcgcacacacaccgauguatt

SEQ ID NO: 464 - DNA, 21 bp, Artificial Sequence
  Description of Artificial Sequence Synthetic oligonucleotide
uacaucggugugugugcgctt

SEQ ID NO: 465 - DNA, 21 bp, Artificial Sequence
  Description of Artificial Sequence Synthetic oligonucleotide
augugcgcacacacagauatt

SEQ ID NO: 466 - DNA, 21 bp, Artificial Sequence
  Description of Artificial Sequence Synthetic oligonucleotide
uaucugugugugcgcacautt

SEQ ID NO: 467 - DNA, 21 bp, Artificial Sequence
  Description of Artificial Sequence Synthetic oligonucleotide
gucacacagagauacgcaatt

SEQ ID NO: 468 - DNA, 21 bp, Artificial Sequence
  Description of Artificial Sequence Synthetic oligonucleotide
uugcguaucucugugugactt

SEQ ID NO: 469 - DNA, 21 bp, Artificial Sequence
  Description of Artificial Sequence Synthetic oligonucleotide
gccaaugcacgcacacauctt

SEQ ID NO: 470 - DNA, 21 bp, Artificial Sequence
  Description of Artificial Sequence Synthetic oligonucleotide
gaugugugcgugcauuggctt

SEQ ID NO: 471 - DNA, 21 bp, Artificial Sequence
  Description of Artificial Sequence Synthetic oligonucleotide
ugaucugugugaacgagaatt

SEQ ID NO: 472 - DNA, 21 bp, Artificial Sequence
  Description of Artificial Sequence Synthetic oligonucleotide
uucucguucacacagaucatt

SEQ ID NO: 473 - DNA, 21 bp, Artificial Sequence
  Description of Artificial Sequence Synthetic oligonucleotide
gcggcccacuguuucgacatt

SEQ ID NO: 474 - DNA, 21 bp, Artificial Sequence
  Description of Artificial Sequence Synthetic oligonucleotide
ugucgaaacagugggccgctt

SEQ ID NO: 475 - DNA, 21 bp, Artificial Sequence
  Description of Artificial Sequence Synthetic oligonucleotide
caaugcacgcacacaucagtt

SEQ ID NO: 476 - DNA, 21 bp, Artificial Sequence
  Description of Artificial Sequence Synthetic oligonucleotide
cugaugugugcgugcauugtt

SEQ ID NO: 477 - DNA, 21 bp, Artificial Sequence
  Description of Artificial Sequence Synthetic oligonucleotide
cacaccgaugugcgcacactt

SEQ ID NO: 478 - DNA, 21 bp, Artificial Sequence
  Description of Artificial Sequence Synthetic oligonucleotide
gugugcgcacaucggugugtt

SEQ ID NO: 479 - DNA, 21 bp, Artificial Sequence
  Description of Artificial Sequence Synthetic oligonucleotide
gcgguuguuuagcucucactt

SEQ ID NO: 480 - DNA, 21 bp, Artificial Sequence
  Description of Artificial Sequence Synthetic oligonucleotide
gugagagcuaaacaaccgctt

SEQ ID NO: 481 - DNA, 21 bp, Artificial Sequence
  Description of Artificial Sequence Synthetic oligonucleotide
accaugcaugguggcgaautt

SEQ ID NO: 482 - DNA, 21 bp, Artificial Sequence
  Description of Artificial Sequence Synthetic oligonucleotide
auucgccaccaugcauggutt

SEQ ID NO: 483 - DNA, 21 bp, Artificial Sequence
  Description of Artificial Sequence Synthetic oligonucleotide
acaucagugcacacggaugtt

SEQ ID NO: 484 - DNA, 21 bp, Artificial Sequence
  Description of Artificial Sequence Synthetic oligonucleotide
cauccgugugcacugaugutt

SEQ ID NO: 485 - DNA, 21 bp, Artificial Sequence
  Description of Artificial Sequence Synthetic oligonucleotide
ucccagcuucacaauaaactt

SEQ ID NO: 486 - DNA, 21 bp, Artificial Sequence
  Description of Artificial Sequence Synthetic oligonucleotide
guuuauugugaagcugggatt

SEQ ID NO: 487 - DNA, 21 bp, Artificial Sequence
  Description of Artificial Sequence Synthetic oligonucleotide
gagauuucaucauggucuctt

SEQ ID NO: 488 - DNA, 21 bp, Artificial Sequence
  Description of Artificial Sequence Synthetic oligonucleotide
gagaccaugaugaaaucuctt

SEQ ID NO: 489 - DNA, 21 bp, Artificial Sequence
  Description of Artificial Sequence Synthetic oligonucleotide
gaaggcgguuguuuagcuctt

SEQ ID NO: 490 - DNA, 21 bp, Artificial Sequence
  Description of Artificial Sequence Synthetic oligonucleotide
gagcuaaacaaccgccuuctt

SEQ ID NO: 491 - DNA, 21 bp, Artificial Sequence
  Description of Artificial Sequence Synthetic oligonucleotide
ccucugaaggcgguuguuutt

SEQ ID NO: 492 - DNA, 21 bp, Artificial Sequence
  Description of Artificial Sequence Synthetic oligonucleotide
aaacaaccgccuucagaggtt

SEQ ID NO: 493 - DNA, 21 bp, Artificial Sequence
  Description of Artificial Sequence Synthetic oligonucleotide
cugugugaacgagaacggctt

SEQ ID NO: 494 - DNA, 21 bp, Artificial Sequence
  Description of Artificial Sequence Synthetic oligonucleotide
gccguucucguucacacagtt

SEQ ID NO: 495 - DNA, 21 bp, Artificial Sequence
  Description of Artificial Sequence Synthetic oligonucleotide
ugcccgaacggacguucuctt

SEQ ID NO: 496 - DNA, 21 bp, Artificial Sequence
  Description of Artificial Sequence Synthetic oligonucleotide
gagaacguccguucgggcatt

SEQ ID NO: 497 - DNA, 21 bp, Artificial Sequence
  Description of Artificial Sequence Synthetic oligonucleotide
uggcaccaaaucccauauatt

SEQ ID NO: 498 - DNA, 21 bp, Artificial Sequence
  Description of Artificial Sequence Synthetic oligonucleotide
uauaugggauuuggugccatt

SEQ ID NO: 499 - DNA, 21 bp, Artificial Sequence
  Description of Artificial Sequence Synthetic oligonucleotide
auacgcaaacacaccgaugtt

SEQ ID NO: 500 - DNA, 21 bp, Artificial Sequence
  Description of Artificial Sequence Synthetic oligonucleotide
caucgguguguuugcguautt

SEQ ID NO: 501 - DNA, 21 bp, Artificial Sequence
  Description of Artificial Sequence Synthetic oligonucleotide
cuguccucugaaggcgguutt

SEQ ID NO: 502 - DNA, 21 bp, Artificial Sequence
  Description of Artificial Sequence Synthetic oligonucleotide
aaccgccuucagaggacagtt

SEQ ID NO: 503 - DNA, 21 bp, Artificial Sequence
  Description of Artificial Sequence Synthetic oligonucleotide
caccaagcgcuccugucggtt

SEQ ID NO: 504 - DNA, 21 bp, Artificial Sequence
  Description of Artificial Sequence Synthetic oligonucleotide
ccgacaggagcgcuuggugtt

SEQ ID NO: 505 - DNA, 21 bp, Artificial Sequence
  Description of Artificial Sequence Synthetic oligonucleotide
ccagcuucacaauaaacggtt

SEQ ID NO: 506 - DNA, 21 bp, Artificial Sequence
  Description of Artificial Sequence Synthetic oligonucleotide
ccguuuauugugaagcuggtt

SEQ ID NO: 507 - DNA, 21 bp, Artificial Sequence
  Description of Artificial Sequence Synthetic oligonucleotide
augccaaugcacgcacacatt

SEQ ID NO: 508 - DNA, 21 bp, Artificial Sequence
  Description of Artificial Sequence Synthetic oligonucleotide
ugugugcgugcauuggcautt

SEQ ID NO: 509 - DNA, 21 bp, Artificial Sequence
  Description of Artificial Sequence Synthetic oligonucleotide
cacacaucagugcacacggtt

SEQ ID NO: 510 - DNA, 21 bp, Artificial Sequence
  Description of Artificial Sequence Synthetic oligonucleotide
ccgugugcacugaugugugtt

SEQ ID NO: 511 - DNA, 21 bp, Artificial Sequence
  Description of Artificial Sequence Synthetic oligonucleotide
gucacggaaggugggagactt

SEQ ID NO: 512 - DNA, 21 bp, Artificial Sequence
  Description of Artificial Sequence Synthetic oligonucleotide
gucucccaccuuccgugactt

SEQ ID NO: 513 - DNA, 21 bp, Artificial Sequence
  Description of Artificial Sequence Synthetic oligonucleotide
acacagagauacgcaaacatt

SEQ ID NO: 514 - DNA, 21 bp, Artificial Sequence
  Description of Artificial Sequence Synthetic oligonucleotide
uguuugcguaucucugugutt

SEQ ID NO: 515 - DNA, 21 bp, Artificial Sequence
  Description of Artificial Sequence Synthetic oligonucleotide
acaugccaaugcacgcacatt

SEQ ID NO: 516 - DNA, 21 bp, Artificial Sequence
  Description of Artificial Sequence Synthetic oligonucleotide
ugugcgugcauuggcaugutt

SEQ ID NO: 517 - DNA, 21 bp, Artificial Sequence
  Description of Artificial Sequence Synthetic oligonucleotide
gcacguacgucccgggcactt

SEQ ID NO: 518 - DNA, 21 bp, Artificial Sequence
  Description of Artificial Sequence Synthetic oligonucleotide
gugcccgggacguacgugctt

SEQ ID NO: 519 - DNA, 21 bp, Artificial Sequence
  Description of Artificial Sequence Synthetic oligonucleotide
gggagugccaagguugucctt

SEQ ID NO: 520 - DNA, 21 bp, Artificial Sequence
  Description of Artificial Sequence Synthetic oligonucleotide
ggacaaccuuggcacuccctt

SEQ ID NO: 521 - DNA, 21 bp, Artificial Sequence
  Description of Artificial Sequence Synthetic oligonucleotide
uauacacauggaugcacgctt

SEQ ID NO: 522 - DNA, 21 bp, Artificial Sequence
  Description of Artificial Sequence Synthetic oligonucleotide
gcgugcauccauguguauatt

SEQ ID NO: 523 - DNA, 21 bp, Artificial Sequence
  Description of Artificial Sequence Synthetic oligonucleotide
guccucugaaggcgguugutt

SEQ ID NO: 524 - DNA, 21 bp, Artificial Sequence
  Description of Artificial Sequence Synthetic oligonucleotide
acaaccgccuucagaggactt

SEQ ID NO: 525 - DNA, 21 bp, Artificial Sequence
  Description of Artificial Sequence Synthetic oligonucleotide
gcccacuguuucgacaaaatt

SEQ ID NO: 526 - DNA, 21 bp, Artificial Sequence
  Description of Artificial Sequence Synthetic oligonucleotide
uuuugucgaaacagugggctt

SEQ ID NO: 527 - DNA, 21 bp, Artificial Sequence
  Description of Artificial Sequence Synthetic oligonucleotide
cacgcacauagagauaugctt

SEQ ID NO: 528 - DNA, 21 bp, Artificial Sequence
  Description of Artificial Sequence Synthetic oligonucleotide
gcauaucucuaugugcgugtt

SEQ ID NO: 529 - DNA, 21 bp, Artificial Sequence
  Description of Artificial Sequence Synthetic oligonucleotide
gccggcgcgccaacgcguutt

SEQ ID NO: 530 - DNA, 21 bp, Artificial Sequence
  Description of Artificial Sequence Synthetic oligonucleotide
aacgcguuggcgcgccggctt

SEQ ID NO: 531 - DNA, 21 bp, Artificial Sequence
  Description of Artificial Sequence Synthetic oligonucleotide
gcucagagaguggacucgatt

SEQ ID NO: 532 - DNA, 21 bp, Artificial Sequence
  Description of Artificial Sequence Synthetic oligonucleotide
ucgaguccacucucugagctt

SEQ ID NO: 533 - DNA, 21 bp, Artificial Sequence
  Description of Artificial Sequence Synthetic oligonucleotide
ccucagcgagcacgacgggtt

SEQ ID NO: 534 - DNA, 21 bp, Artificial Sequence
  Description of Artificial Sequence Synthetic oligonucleotide
cccgucgugcucgcugaggtt

SEQ ID NO: 535 - DNA, 21 bp, Artificial Sequence
  Description of Artificial Sequence Synthetic oligonucleotide
uucgugcgcuucucauuggtt

SEQ ID NO: 536 - DNA, 21 bp, Artificial Sequence
  Description of Artificial Sequence Synthetic oligonucleotide
ccaaugagaagcgcacgaatt

SEQ ID NO: 537 - DNA, 21 bp, Artificial Sequence
  Description of Artificial Sequence Synthetic oligonucleotide
gaaagccaaggcugcgucgtt

SEQ ID NO: 538 - DNA, 21 bp, Artificial Sequence
  Description of Artificial Sequence Synthetic oligonucleotide
cgacgcagccuuggcuuuctt

SEQ ID NO: 539 - DNA, 21 bp, Artificial Sequence
  Description of Artificial Sequence Synthetic oligonucleotide
gaccagcuccaguccuauatt

SEQ ID NO: 540 - DNA, 21 bp, Artificial Sequence
  Description of Artificial Sequence Synthetic oligonucleotide
uauaggacuggagcugguctt

SEQ ID NO: 541 - DNA, 21 bp, Artificial Sequence
  Description of Artificial Sequence Synthetic oligonucleotide
ugcgcacacacaccgaugutt

SEQ ID NO: 542 - DNA, 21 bp, Artificial Sequence
  Description of Artificial Sequence Synthetic oligonucleotide
acaucggugugugugcgcatt

SEQ ID NO: 543 - DNA, 21 bp, Artificial Sequence
  Description of Artificial Sequence Synthetic oligonucleotide
agagauuucaucauggucutt

SEQ ID NO: 544 - DNA, 21 bp, Artificial Sequence
  Description of Artificial Sequence Synthetic oligonucleotide
agaccaugaugaaaucucutt

SEQ ID NO: 545 - DNA, 21 bp, Artificial Sequence
  Description of Artificial Sequence Synthetic oligonucleotide
caaauaucacggaguacautt

SEQ ID NO: 546 - DNA, 21 bp, Artificial Sequence
  Description of Artificial Sequence Synthetic oligonucleotide
auguacuccgugauauuugtt

SEQ ID NO: 547 - DNA, 21 bp, Artificial Sequence
  Description of Artificial Sequence Synthetic oligonucleotide
acgcacacaucagugcacatt

SEQ ID NO: 548 - DNA, 21 bp, Artificial Sequence
  Description of Artificial Sequence Synthetic oligonucleotide
ugugcacugaugugugcgutt

SEQ ID NO: 549 - DNA, 21 bp, Artificial Sequence
  Description of Artificial Sequence Synthetic oligonucleotide
caccaccaaccacgacauctt

SEQ ID NO: 550 - DNA, 21 bp, Artificial Sequence
  Description of Artificial Sequence Synthetic oligonucleotide
gaugucgugguugguggugtt

SEQ ID NO: 551 - DNA, 21 bp, Artificial Sequence
  Description of Artificial Sequence Synthetic oligonucleotide
uggacucgaugccaucccutt

SEQ ID NO: 552 - DNA, 21 bp, Artificial Sequence
  Description of Artificial Sequence Synthetic oligonucleotide
agggauggcaucgaguccatt

SEQ ID NO: 553 - DNA, 21 bp, Artificial Sequence
  Description of Artificial Sequence Synthetic oligonucleotide
cucugccugcccgaacggatt

SEQ ID NO: 554 - DNA, 21 bp, Artificial Sequence
  Description of Artificial Sequence Synthetic oligonucleotide
uccguucgggcaggcagagtt

SEQ ID NO: 555 - DNA, 21 bp, Artificial Sequence
  Description of Artificial Sequence Synthetic oligonucleotide
uucugugccggcuacucggtt

SEQ ID NO: 556 - DNA, 21 bp, Artificial Sequence
  Description of Artificial Sequence Synthetic oligonucleotide
ccgaguagccggcacagaatt

SEQ ID NO: 557 - DNA, 21 bp, Artificial Sequence
  Description of Artificial Sequence Synthetic oligonucleotide
cacguacgucccgggcacctt

SEQ ID NO: 558 - DNA, 21 bp, Artificial Sequence
  Description of Artificial Sequence Synthetic oligonucleotide
ggugcccgggacguacgugtt

SEQ ID NO: 559 - DNA, 21 bp, Artificial Sequence
  Description of Artificial Sequence Synthetic oligonucleotide
ccucugccugcccgaacggtt

SEQ ID NO: 560 - DNA, 21 bp, Artificial Sequence
  Description of Artificial Sequence Synthetic oligonucleotide
ccguucgggcaggcagaggtt

SEQ ID NO: 561 - DNA, 21 bp, Artificial Sequence
  Description of Artificial Sequence Synthetic oligonucleotide
gcgcgccaacgcguuccugtt

SEQ ID NO: 562 - DNA, 21 bp, Artificial Sequence
  Description of Artificial Sequence Synthetic oligonucleotide
caggaacgcguuggcgcgctt

SEQ ID NO: 563 - DNA, 21 bp, Artificial Sequence
  Description of Artificial Sequence Synthetic oligonucleotide
ggcccacuguuucgacaaatt

SEQ ID NO: 564 - DNA, 21 bp, Artificial Sequence
  Description of Artificial Sequence Synthetic oligonucleotide
uuugucgaaacagugggcctt

SEQ ID NO: 565 - DNA, 21 bp, Artificial Sequence
  Description of Artificial Sequence Synthetic oligonucleotide
agaucuucaaggacgcggatt

SEQ ID NO: 566 - DNA, 21 bp, Artificial Sequence
  Description of Artificial Sequence Synthetic oligonucleotide
uccgcguccuugaagaucutt

SEQ ID NO: 567 - DNA, 21 bp, Artificial Sequence
  Description of Artificial Sequence Synthetic oligonucleotide
auguauuucucccuucgcutt

SEQ ID NO: 568 - DNA, 21 bp, Artificial Sequence
  Description of Artificial Sequence Synthetic oligonucleotide
agcgaagggagaaauacautt

SEQ ID NO: 569 - DNA, 21 bp, Artificial Sequence
  Description of Artificial Sequence Synthetic oligonucleotide
gauaugcacacaccgaugutt

SEQ ID NO: 570 - DNA, 21 bp, Artificial Sequence
  Description of Artificial Sequence Synthetic oligonucleotide
acaucggugugugcauauctt

SEQ ID NO: 571 - DNA, 21 bp, Artificial Sequence
  Description of Artificial Sequence Synthetic oligonucleotide
uacugcagugaccacacggtt

SEQ ID NO: 572 - DNA, 21 bp, Artificial Sequence
  Description of Artificial Sequence Synthetic oligonucleotide
ccguguggucacugcaguatt

SEQ ID NO: 573 - DNA, 21 bp, Artificial Sequence
  Description of Artificial Sequence Synthetic oligonucleotide
ccagggcugcgcaaccgugtt

SEQ ID NO: 574 - DNA, 21 bp, Artificial Sequence
  Description of Artificial Sequence Synthetic oligonucleotide
cacgguugcgcagcccuggtt

SEQ ID NO: 575 - DNA, 21 bp, Artificial Sequence
  Description of Artificial Sequence Synthetic oligonucleotide
caguccuauaucugcuucutt

SEQ ID NO: 576 - DNA, 21 bp, Artificial Sequence
  Description of Artificial Sequence Synthetic oligonucleotide
agaagcagauauaggacugtt

SEQ ID NO: 577 - DNA, 21 bp, Artificial Sequence
  Description of Artificial Sequence Synthetic oligonucleotide
ccugcccgaacggacguuctt

SEQ ID NO: 578 - DNA, 21 bp, Artificial Sequence
  Description of Artificial Sequence Synthetic oligonucleotide
gaacguccguucgggcaggtt

SEQ ID NO: 579 - DNA, 21 bp, Artificial Sequence
  Description of Artificial Sequence Synthetic oligonucleotide
cacgcaucacuaaaugcaatt

SEQ ID NO: 580 - DNA, 21 bp, Artificial Sequence
  Description of Artificial Sequence Synthetic oligonucleotide
uugcauuuagugaugcgugtt

SEQ ID NO: 581 - DNA, 21 bp, Artificial Sequence
  Description of Artificial Sequence Synthetic oligonucleotide
ugcacacaccgaugugcgctt

SEQ ID NO: 582 - DNA, 21 bp, Artificial Sequence
  Description of Artificial Sequence Synthetic oligonucleotide
gcgcacaucggugugugcatt

SEQ ID NO: 583 - DNA, 21 bp, Artificial Sequence
  Description of Artificial Sequence Synthetic oligonucleotide
cagcacguacgucccgggctt

SEQ ID NO: 584 - DNA, 21 bp, Artificial Sequence
  Description of Artificial Sequence Synthetic oligonucleotide
gcccgggacguacgugcugtt

SEQ ID NO: 585 - DNA, 21 bp, Artificial Sequence
  Description of Artificial Sequence Synthetic oligonucleotide
gugcgcuucucauuggucatt

SEQ ID NO: 586 - DNA, 21 bp, Artificial Sequence
  Description of Artificial Sequence Synthetic oligonucleotide
ugaccaaugagaagcgcactt

SEQ ID NO: 587 - DNA, 21 bp, Artificial Sequence
  Description of Artificial Sequence Synthetic oligonucleotide
aacggacguucucugagagtt

SEQ ID NO: 588 - DNA, 21 bp, Artificial Sequence
  Description of Artificial Sequence Synthetic oligonucleotide
cucucagagaacguccguutt

SEQ ID NO: 589 - DNA, 21 bp, Artificial Sequence
  Description of Artificial Sequence Synthetic oligonucleotide
gaucuucaaggacgcggagtt

SEQ ID NO: 590 - DNA, 21 bp, Artificial Sequence
  Description of Artificial Sequence Synthetic oligonucleotide
cuccgcguccuugaagauctt

SEQ ID NO: 591 - DNA, 21 bp, Artificial Sequence
  Description of Artificial Sequence Synthetic oligonucleotide
ccauggcagguccuguugutt

SEQ ID NO: 592 - DNA, 21 bp, Artificial Sequence
  Description of Artificial Sequence Synthetic oligonucleotide
acaacaggaccugccauggtt

SEQ ID NO: 593 - DNA, 21 bp, Artificial Sequence
  Description of Artificial Sequence Synthetic oligonucleotide
cuaugaacuacagccguggtt

SEQ ID NO: 594 - DNA, 21 bp, Artificial Sequence
  Description of Artificial Sequence Synthetic oligonucleotide
ccacggcuguaguucauagtt

SEQ ID NO: 595 - DNA, 21 bp, Artificial Sequence
  Description of Artificial Sequence Synthetic oligonucleotide
uacgcaaacacaccgaugctt

SEQ ID NO: 596 - DNA, 21 bp, Artificial Sequence
  Description of Artificial Sequence Synthetic oligonucleotide
gcaucgguguguuugcguatt

SEQ ID NO: 597 - DNA, 21 bp, Artificial Sequence
  Description of Artificial Sequence Synthetic oligonucleotide
caaggcugcgucgaacugutt

SEQ ID NO: 598 - DNA, 21 bp, Artificial Sequence
  Description of Artificial Sequence Synthetic oligonucleotide
acaguucgacgcagccuugtt

SEQ ID NO: 599 - DNA, 21 bp, Artificial Sequence
  Description of Artificial Sequence Synthetic oligonucleotide
agauaugcacacaccgaugtt

SEQ ID NO: 600 - DNA, 21 bp, Artificial Sequence
  Description of Artificial Sequence Synthetic oligonucleotide
caucggugugugcauaucutt

SEQ ID NO: 601 - DNA, 21 bp, Artificial Sequence
  Description of Artificial Sequence Synthetic oligonucleotide
cugcgucgaacuguccuggtt

SEQ ID NO: 602 - DNA, 21 bp, Artificial Sequence
  Description of Artificial Sequence Synthetic oligonucleotide
ccaggacaguucgacgcagtt

SEQ ID NO: 603 - DNA, 21 bp, Artificial Sequence
  Description of Artificial Sequence Synthetic oligonucleotide
augcgcacacacaccgaugtt

SEQ ID NO: 604 - DNA, 21 bp, Artificial Sequence
  Description of Artificial Sequence Synthetic oligonucleotide
caucggugugugugcgcautt

SEQ ID NO: 605 - DNA, 21 bp, Artificial Sequence
  Description of Artificial Sequence Synthetic oligonucleotide
ucugccugcccgaacggactt

SEQ ID NO: 606 - DNA, 21 bp, Artificial Sequence
  Description of Artificial Sequence Synthetic oligonucleotide
guccguucgggcaggcagatt

SEQ ID NO: 607 - DNA, 21 bp, Artificial Sequence
  Description of Artificial Sequence Synthetic oligonucleotide
gacuccggcaagcacggcutt

SEQ ID NO: 608 - DNA, 21 bp, Artificial Sequence
  Description of Artificial Sequence Synthetic oligonucleotide
agccgugcuugccggaguctt

SEQ ID NO: 609 - DNA, 21 bp, Artificial Sequence
  Description of Artificial Sequence Synthetic oligonucleotide
gacgcuggccuucgugcgctt

SEQ ID NO: 610 - DNA, 21 bp, Artificial Sequence
  Description of Artificial Sequence Synthetic oligonucleotide
gcgcacgaaggccagcguctt

SEQ ID NO: 611 - DNA, 21 bp, Artificial Sequence
  Description of Artificial Sequence Synthetic oligonucleotide
cgcacacacaccgauguactt

SEQ ID NO: 612 - DNA, 21 bp, Artificial Sequence
  Description of Artificial Sequence Synthetic oligonucleotide
guacaucggugugugugcgtt

SEQ ID NO: 613 - DNA, 21 bp, Artificial Sequence
  Description of Artificial Sequence Synthetic oligonucleotide
agauuucaucauggucucctt

SEQ ID NO: 614 - DNA, 21 bp, Artificial Sequence
  Description of Artificial Sequence Synthetic oligonucleotide
ggagaccaugaugaaaucutt

SEQ ID NO: 615 - DNA, 21 bp, Artificial Sequence
  Description of Artificial Sequence Synthetic oligonucleotide
aaggcugcgucgaacuguctt

SEQ ID NO: 616 - DNA, 21 bp, Artificial Sequence
  Description of Artificial Sequence Synthetic oligonucleotide
gacaguucgacgcagccuutt

SEQ ID NO: 617 - DNA, 21 bp, Artificial Sequence
  Description of Artificial Sequence Synthetic oligonucleotide
ugcgucuccuccgcacacctt

SEQ ID NO: 618 - DNA, 21 bp, Artificial Sequence
  Description of Artificial Sequence Synthetic oligonucleotide
ggugugcggaggagacgcatt

SEQ ID NO: 619 - DNA, 21 bp, Artificial Sequence
  Description of Artificial Sequence Synthetic oligonucleotide
aauaaacggcugcgucucctt

SEQ ID NO: 620 - DNA, 21 bp, Artificial Sequence
  Description of Artificial Sequence Synthetic oligonucleotide
ggagacgcagccguuuauutt

SEQ ID NO: 621 - DNA, 21 bp, Artificial Sequence
  Description of Artificial Sequence Synthetic oligonucleotide
auaugcacacacacggaugtt

SEQ ID NO: 622 - DNA, 21 bp, Artificial Sequence
  Description of Artificial Sequence Synthetic oligonucleotide
cauccgugugugugcauautt

SEQ ID NO: 623 - DNA, 21 bp, Artificial Sequence
  Description of Artificial Sequence Synthetic oligonucleotide
aaggcgguuguuuagcucutt

SEQ ID NO: 624 - DNA, 21 bp, Artificial Sequence
  Description of Artificial Sequence Synthetic oligonucleotide
agagcuaaacaaccgccuutt

SEQ ID NO: 625 - DNA, 21 bp, Artificial Sequence
  Description of Artificial Sequence Synthetic oligonucleotide
acgcaucacuaaaugcaagtt

SEQ ID NO: 626 - DNA, 21 bp, Artificial Sequence
  Description of Artificial Sequence Synthetic oligonucleotide
cuugcauuuagugaugcgutt

SEQ ID NO: 627 - DNA, 21 bp, Artificial Sequence
  Description of Artificial Sequence Synthetic oligonucleotide
cugccugcccgaacggacgtt

SEQ ID NO: 628 - DNA, 21 bp, Artificial Sequence
  Description of Artificial Sequence Synthetic oligonucleotide
cguccguucgggcaggcagtt

SEQ ID NO: 629 - DNA, 21 bp, Artificial Sequence
  Description of Artificial Sequence Synthetic oligonucleotide
cggcccacuguuucgacaatt

SEQ ID NO: 630 - DNA, 21 bp, Artificial Sequence
  Description of Artificial Sequence Synthetic oligonucleotide
uugucgaaacagugggccgtt

SEQ ID NO: 631 - DNA, 21 bp, Artificial Sequence
  Description of Artificial Sequence Synthetic oligonucleotide
cagggcugcgcaaccguggtt

SEQ ID NO: 632 - DNA, 21 bp, Artificial Sequence
  Description of Artificial Sequence Synthetic oligonucleotide
ccacgguugcgcagcccugtt

SEQ ID NO: 633 - DNA, 21 bp, Artificial Sequence
  Description of Artificial Sequence Synthetic oligonucleotide
uggucacacagagauacgctt

SEQ ID NO: 634 - DNA, 21 bp, Artificial Sequence
  Description of Artificial Sequence Synthetic oligonucleotide
gcguaucucugugugaccatt

SEQ ID NO: 635 - DNA, 21 bp, Artificial Sequence
  Description of Artificial Sequence Synthetic oligonucleotide
cuccugucggugccacgagtt

SEQ ID NO: 636 - DNA, 21 bp, Artificial Sequence
  Description of Artificial Sequence Synthetic oligonucleotide
cucguggcaccgacaggagtt

SEQ ID NO: 637 - DNA, 21 bp, Artificial Sequence
  Description of Artificial Sequence Synthetic oligonucleotide
caaggaccagcuccagucctt

SEQ ID NO: 638 - DNA, 21 bp, Artificial Sequence
  Description of Artificial Sequence Synthetic oligonucleotide
ggacuggagcugguccuugtt

SEQ ID NO: 639 - DNA, 21 bp, Artificial Sequence
  Description of Artificial Sequence Synthetic oligonucleotide
uucucauuggucagcggcutt

SEQ ID NO: 640 - DNA, 21 bp, Artificial Sequence
  Description of Artificial Sequence Synthetic oligonucleotide
agccgcugaccaaugagaatt

SEQ ID NO: 641 - DNA, 21 bp, Artificial Sequence
  Description of Artificial Sequence Synthetic oligonucleotide
gagaucuucaaggacgcggtt

SEQ ID NO: 642 - DNA, 21 bp, Artificial Sequence
  Description of Artificial Sequence Synthetic oligonucleotide
ccgcguccuugaagaucuctt

SEQ ID NO: 643 - DNA, 21 bp, Artificial Sequence
  Description of Artificial Sequence Synthetic oligonucleotide
agagaguggacucgaugcctt

SEQ ID NO: 644 - DNA, 21 bp, Artificial Sequence
  Description of Artificial Sequence Synthetic oligonucleotide
ggcaucgaguccacucucutt

SEQ ID NO: 645 - DNA, 21 bp, Artificial Sequence
  Description of Artificial Sequence Synthetic oligonucleotide
cucccaguacaucgaguggtt

SEQ ID NO: 646 - DNA, 21 bp, Artificial Sequence
  Description of Artificial Sequence Synthetic oligonucleotide
ccacucgauguacugggagtt

SEQ ID NO: 647 - DNA, 21 bp, Artificial Sequence
  Description of Artificial Sequence Synthetic oligonucleotide
agucagggacacacgcauctt

SEQ ID NO: 648 - DNA, 21 bp, Artificial Sequence
  Description of Artificial Sequence Synthetic oligonucleotide
gaugcgugugucccugacutt

SEQ ID NO: 649 - DNA, 21 bp, Artificial Sequence
  Description of Artificial Sequence Synthetic oligonucleotide
ccaucccugcagggccguctt

SEQ ID NO: 650 - DNA, 21 bp, Artificial Sequence
  Description of Artificial Sequence Synthetic oligonucleotide
gacggcccugcagggauggtt

SEQ ID NO: 651 - DNA, 21 bp, Artificial Sequence
  Description of Artificial Sequence Synthetic oligonucleotide
agucuucguaacccaggagtt

SEQ ID NO: 652 - DNA, 21 bp, Artificial Sequence
  Description of Artificial Sequence Synthetic oligonucleotide
cuccuggguuacgaagacutt

SEQ ID NO: 653 - DNA, 21 bp, Artificial Sequence
  Description of Artificial Sequence Synthetic oligonucleotide
caagcgcuccugucggugctt

SEQ ID NO: 654 - DNA, 21 bp, Artificial Sequence
  Description of Artificial Sequence Synthetic oligonucleotide
gcaccgacaggagcgcuugtt

SEQ ID NO: 655 - DNA, 21 bp, Artificial Sequence
  Description of Artificial Sequence Synthetic oligonucleotide
gguccucacugaccaugugtt

SEQ ID NO: 656 - DNA, 21 bp, Artificial Sequence
  Description of Artificial Sequence Synthetic oligonucleotide
cacauggucagugaggacctt

SEQ ID NO: 657 - DNA, 21 bp, Artificial Sequence
  Description of Artificial Sequence Synthetic oligonucleotide
aggcugcgucgaacugucctt

SEQ ID NO: 658 - DNA, 21 bp, Artificial Sequence
  Description of Artificial Sequence Synthetic oligonucleotide
ggacaguucgacgcagccutt

SEQ ID NO: 659 - DNA, 21 bp, Artificial Sequence
  Description of Artificial Sequence Synthetic oligonucleotide
ggacacacgcaucacuaaatt

SEQ ID NO: 660 - DNA, 21 bp, Artificial Sequence
  Description of Artificial Sequence Synthetic oligonucleotide
uuuagugaugcgugugucctt

SEQ ID NO: 661 - DNA, 21 bp, Artificial Sequence
  Description of Artificial Sequence Synthetic oligonucleotide
ugcacacacaccgaugcugtt

SEQ ID NO: 662 - DNA, 21 bp, Artificial Sequence
  Description of Artificial Sequence Synthetic oligonucleotide
cagcaucggugugugugcatt

SEQ ID NO: 663 - DNA, 21 bp, Artificial Sequence
  Description of Artificial Sequence Synthetic oligonucleotide
acugaaaugaacccucacatt

SEQ ID NO: 664 - DNA, 21 bp, Artificial Sequence
  Description of Artificial Sequence Synthetic oligonucleotide
ugugaggguucauuucagutt

SEQ ID NO: 665 - DNA, 21 bp, Artificial Sequence
  Description of Artificial Sequence Synthetic oligonucleotide
caguugaauauccauguggtt

SEQ ID NO: 666 - DNA, 21 bp, Artificial Sequence
  Description of Artificial Sequence Synthetic oligonucleotide
ccacauggauauucaacugtt

SEQ ID NO: 667 - DNA, 21 bp, Artificial Sequence
  Description of Artificial Sequence Synthetic oligonucleotide
ugagcaguacugcagugactt

SEQ ID NO: 668 - DNA, 21 bp, Artificial Sequence
  Description of Artificial Sequence Synthetic oligonucleotide
gucacugcaguacugcucatt

SEQ ID NO: 669 - DNA, 21 bp, Artificial Sequence
  Description of Artificial Sequence Synthetic oligonucleotide
gcugugagcaguacugcagtt

SEQ ID NO: 670 - DNA, 21 bp, Artificial Sequence
  Description of Artificial Sequence Synthetic oligonucleotide
cugcaguacugcucacagctt

SEQ ID NO: 671 - DNA, 21 bp, Artificial Sequence
  Description of Artificial Sequence Synthetic oligonucleotide
ggcugugagcaguacugcatt

SEQ ID NO: 672 - DNA, 21 bp, Artificial Sequence
  Description of Artificial Sequence Synthetic oligonucleotide
ugcaguacugcucacagcctt

SEQ ID NO: 673 - DNA, 21 bp, Artificial Sequence
  Description of Artificial Sequence Synthetic oligonucleotide
ggcugugagcaguacugcatt

SEQ ID NO: 674 - DNA, 21 bp, Artificial Sequence
  Description of Artificial Sequence Synthetic oligonucleotide
ugcaguacugcucacagcctt

SEQ ID NO: 675 - DNA, 21 bp, Artificial Sequence
  Description of Artificial Sequence Synthetic oligonucleotide
gugagcaguacugcagugatt

SEQ ID NO: 676 - DNA, 21 bp, Artificial Sequence
  Description of Artificial Sequence Synthetic oligonucleotide
ucacugcaguacugcucactt

SEQ ID NO: 677 - DNA, 21 bp, Artificial Sequence
  Description of Artificial Sequence Synthetic oligonucleotide
cccacaguugaauauccautt

SEQ ID NO: 678 - DNA, 21 bp, Artificial Sequence
  Description of Artificial Sequence Synthetic oligonucleotide
auggauauucaacugugggtt

SEQ ID NO: 679 - DNA, 21 bp, Artificial Sequence
  Description of Artificial Sequence Synthetic oligonucleotide
cugugagcaguacugcagutt

SEQ ID NO: 680 - DNA, 21 bp, Artificial Sequence
  Description of Artificial Sequence Synthetic oligonucleotide
acugcaguacugcucacagtt

SEQ ID NO: 681 - DNA, 21 bp, Artificial Sequence
  Description of Artificial Sequence Synthetic oligonucleotide
ccacaguugaauauccaugtt

SEQ ID NO: 682 - DNA, 21 bp, Artificial Sequence
  Description of Artificial Sequence Synthetic oligonucleotide
cauggauauucaacuguggtt

SEQ ID NO: 683 - DNA, 21 bp, Artificial Sequence
  Description of Artificial Sequence Synthetic oligonucleotide
acauguucugugccggcuatt

SEQ ID NO: 684 - DNA, 21 bp, Artificial Sequence
  Description of Artificial Sequence Synthetic oligonucleotide
uagccggcacagaacaugutt

SEQ ID NO: 685 - DNA, 21 bp, Artificial Sequence
  Description of Artificial Sequence Synthetic oligonucleotide
ucgaggaggcccgggagautt

SEQ ID NO: 686 - DNA, 21 bp, Artificial Sequence
  Description of Artificial Sequence Synthetic oligonucleotide
aucucccgggccuccucgatt

SEQ ID NO: 687 - DNA, 21 bp, Artificial Sequence
  Description of Artificial Sequence Synthetic oligonucleotide
gagcaguacugcagugacctt

SEQ ID NO: 688 - DNA, 21 bp, Artificial Sequence
  Description of Artificial Sequence Synthetic oligonucleotide
ggucacugcaguacugcuctt

SEQ ID NO: 689 - DNA, 21 bp, Artificial Sequence
  Description of Artificial Sequence Synthetic oligonucleotide
gcugugagcaguacugcagtt

SEQ ID NO: 690 - DNA, 21 bp, Artificial Sequence
  Description of Artificial Sequence Synthetic oligonucleotide
cugcaguacugcucacagctt

SEQ ID NO: 691 - DNA, 21 bp, Artificial Sequence
  Description of Artificial Sequence Synthetic oligonucleotide
cacaguugaauauccaugutt

SEQ ID NO: 692 - DNA, 21 bp, Artificial Sequence
  Description of Artificial Sequence Synthetic oligonucleotide
acauggauauucaacugugtt

SEQ ID NO: 693 - DNA, 21 bp, Artificial Sequence
  Description of Artificial Sequence Synthetic oligonucleotide
cauguucugugccggcuactt

SEQ ID NO: 694 - DNA, 21 bp, Artificial Sequence
  Description of Artificial Sequence Synthetic oligonucleotide
guagccggcacagaacaugtt

SEQ ID NO: 695 - DNA, 21 bp, Artificial Sequence
  Description of Artificial Sequence Synthetic oligonucleotide
cgaggaggcccgggagauctt

SEQ ID NO: 696 - DNA, 21 bp, Artificial Sequence
  Description of Artificial Sequence Synthetic oligonucleotide
gaucucccgggccuccucgtt

SEQ ID NO: 697 - DNA, 21 bp, Artificial Sequence
  Description of Artificial Sequence Synthetic oligonucleotide
cauguucugugccggcuactt

SEQ ID NO: 698 - DNA, 21 bp, Artificial Sequence
  Description of Artificial Sequence Synthetic oligonucleotide
guagccggcacagaacaugtt

SEQ ID NO: 699 - DNA, 21 bp, Artificial Sequence
  Description of Artificial Sequence Synthetic oligonucleotide
cccacaguugaauauccautt

SEQ ID NO: 700 - DNA, 21 bp, Artificial Sequence
  Description of Artificial Sequence Synthetic oligonucleotide
auggauauucaacugugggtt

SEQ ID NO: 701 - DNA, 21 bp, Artificial Sequence
  Description of Artificial Sequence Synthetic oligonucleotide
acauguucugugccggcuatt

SEQ ID NO: 702 - DNA, 21 bp, Artificial Sequence
  Description of Artificial Sequence Synthetic oligonucleotide
uagccggcacagaacaugutt

SEQ ID NO: 703 - DNA, 21 bp, Artificial Sequence
  Description of Artificial Sequence Synthetic oligonucleotide
ugugagcaguacugcagugtt

SEQ ID NO: 704 - DNA, 21 bp, Artificial Sequence
  Description of Artificial Sequence Synthetic oligonucleotide
cacugcaguacugcucacatt

SEQ ID NO: 705 - DNA, 21 bp, Artificial Sequence
  Description of Artificial Sequence Synthetic oligonucleotide
caguugaauauccauguggtt

SEQ ID NO: 706 - DNA, 21 bp, Artificial Sequence
  Description of Artificial Sequence Synthetic oligonucleotide
ccacauggauauucaacugtt

SEQ ID NO: 707 - DNA, 21 bp, Artificial Sequence
  Description of Artificial Sequence Synthetic oligonucleotide
ggccagcugcuggaccgugtt

SEQ ID NO: 708 - DNA, 21 bp, Artificial Sequence
  Description of Artificial Sequence Synthetic oligonucleotide
cacgguccagcagcuggcctt

SEQ ID NO: 709 - DNA, 21 bp, Artificial Sequence
  Description of Artificial Sequence Synthetic oligonucleotide
cacaguugaauauccaugutt

SEQ ID NO: 710 - DNA, 21 bp, Artificial Sequence
  Description of Artificial Sequence Synthetic oligonucleotide
acauggauauucaacugugtt

SEQ ID NO: 711 - DNA, 21 bp, Artificial Sequence
  Description of Artificial Sequence Synthetic oligonucleotide
gugagcaguacugcagugatt

SEQ ID NO: 712 - DNA, 21 bp, Artificial Sequence
  Description of Artificial Sequence Synthetic oligonucleotide
ucacugcaguacugcucactt

SEQ ID NO: 713 - DNA, 21 bp, Artificial Sequence
  Description of Artificial Sequence Synthetic oligonucleotide
auguucugugccggcuacutt

SEQ ID NO: 714 - DNA, 21 bp, Artificial Sequence
  Description of Artificial Sequence Synthetic oligonucleotide
aguagccggcacagaacautt

SEQ ID NO: 715 - DNA, 21 bp, Artificial Sequence
  Description of Artificial Sequence Synthetic oligonucleotide
ccacaguugaauauccaugtt

SEQ ID NO: 716 - DNA, 21 bp, Artificial Sequence
  Description of Artificial Sequence Synthetic oligonucleotide
cauggauauucaacuguggtt

SEQ ID NO: 717 - DNA, 21 bp, Artificial Sequence
  Description of Artificial Sequence Synthetic oligonucleotide
acaguugaauauccaugugtt

SEQ ID NO: 718 - DNA, 21 bp, Artificial Sequence
  Description of Artificial Sequence Synthetic oligonucleotide
cacauggauauucaacugutt

SEQ ID NO: 719 - DNA, 21 bp, Artificial Sequence
  Description of Artificial Sequence Synthetic oligonucleotide
auguucugugccggcuacutt

SEQ ID NO: 720 - DNA, 21 bp, Artificial Sequence
  Description of Artificial Sequence Synthetic oligonucleotide
aguagccggcacagaacautt

SEQ ID NO: 721 - DNA, 21 bp, Artificial Sequence
  Description of Artificial Sequence Synthetic oligonucleotide
cagcugcuggaccguggcgtt

SEQ ID NO: 722 - DNA, 21 bp, Artificial Sequence
  Description of Artificial Sequence Synthetic oligonucleotide
cgccacgguccagcagcugtt

SEQ ID NO: 723 - DNA, 21 bp, Artificial Sequence
  Description of Artificial Sequence Synthetic oligonucleotide
ggccagcugcuggaccgugtt

SEQ ID NO: 724 - DNA, 21 bp, Artificial Sequence
  Description of Artificial Sequence Synthetic oligonucleotide
cacgguccagcagcuggcctt

SEQ ID NO: 725 - DNA, 21 bp, Artificial Sequence
  Description of Artificial Sequence Synthetic oligonucleotide
ucgaggaggcccgggagautt

SEQ ID NO: 726 - DNA, 21 bp, Artificial Sequence
  Description of Artificial Sequence Synthetic oligonucleotide
aucucccgggccuccucgatt

SEQ ID NO: 727 - DNA, 21 bp, Artificial Sequence
  Description of Artificial Sequence Synthetic oligonucleotide
gccagcugcuggaccguggtt

SEQ ID NO: 728 - DNA, 21 bp, Artificial Sequence
  Description of Artificial Sequence Synthetic oligonucleotide
ccacgguccagcagcuggctt

SEQ ID NO: 729 - DNA, 21 bp, Artificial Sequence
  Description of Artificial Sequence Synthetic oligonucleotide
gggccagcugcuggaccgutt

SEQ ID NO: 730 - DNA, 21 bp, Artificial Sequence
  Description of Artificial Sequence Synthetic oligonucleotide
acgguccagcagcuggccctt

SEQ ID NO: 731 - DNA, 21 bp, Artificial Sequence
  Description of Artificial Sequence Synthetic oligonucleotide
uucgaggaggcccgggagatt

SEQ ID NO: 732 - DNA, 21 bp, Artificial Sequence
  Description of Artificial Sequence Synthetic oligonucleotide
ucucccgggccuccucgaatt

SEQ ID NO: 733 - DNA, 21 bp, Artificial Sequence
  Description of Artificial Sequence Synthetic oligonucleotide
agcugcuggaccguggcgctt

SEQ ID NO: 734 - DNA, 21 bp, Artificial Sequence
  Description of Artificial Sequence Synthetic oligonucleotide
gcgccacgguccagcagcutt

SEQ ID NO: 735 - DNA, 21 bp, Artificial Sequence
  Description of Artificial Sequence Synthetic oligonucleotide
agcugcuggaccguggcgctt

SEQ ID NO: 736 - DNA, 21 bp, Artificial Sequence
  Description of Artificial Sequence Synthetic oligonucleotide
gcgccacgguccagcagcutt

SEQ ID NO: 737 - DNA, 21 bp, Artificial Sequence
  Description of Artificial Sequence Synthetic oligonucleotide
cagcugcuggaccguggcgtt

SEQ ID NO: 738 - DNA, 21 bp, Artificial Sequence
  Description of Artificial Sequence Synthetic oligonucleotide
cgccacgguccagcagcugtt

SEQ ID NO: 739 - DNA, 21 bp, Artificial Sequence
  Description of Artificial Sequence Synthetic oligonucleotide
gggccagcugcuggaccgutt

SEQ ID NO: 740 - DNA, 21 bp, Artificial Sequence
  Description of Artificial Sequence Synthetic oligonucleotide
acgguccagcagcuggccctt

SEQ ID NO: 741 - DNA, 21 bp, Artificial Sequence
  Description of Artificial Sequence Synthetic oligonucleotide
uucgaggaggcccgggagatt

SEQ ID NO: 742 - DNA, 21 bp, Artificial Sequence
  Description of Artificial Sequence Synthetic oligonucleotide
ucucccgggccuccucgaatt

SEQ ID NO: 743 - DNA, 21 bp, Artificial Sequence
  Description of Artificial Sequence Synthetic oligonucleotide
cgaggaggcccgggagauctt

SEQ ID NO: 744 - DNA, 21 bp, Artificial Sequence
  Description of Artificial Sequence Synthetic oligonucleotide
gaucucccgggccuccucgtt

SEQ ID NO: 745 - DNA, 21 bp, Artificial Sequence
  Description of Artificial Sequence Synthetic oligonucleotide
ccagcugcuggaccguggctt

SEQ ID NO: 746 - DNA, 21 bp, Artificial Sequence
  Description of Artificial Sequence Synthetic oligonucleotide
gccacgguccagcagcuggtt

SEQ ID NO: 747 - DNA, 21 bp, Artificial Sequence
  Description of Artificial Sequence Synthetic oligonucleotide
gccagcugcuggaccguggtt

SEQ ID NO: 748 - DNA, 21 bp, Artificial Sequence
  Description of Artificial Sequence Synthetic oligonucleotide
ccacgguccagcagcuggctt

SEQ ID NO: 749 - DNA, 21 bp, Artificial Sequence
  Description of Artificial Sequence Synthetic oligonucleotide
ccagcugcuggaccguggctt

SEQ ID NO: 750 - DNA, 21 bp, Artificial Sequence
  Description of Artificial Sequence Synthetic oligonucleotide
gccacgguccagcagcuggtt

SEQ ID NO: 751 - DNA, 21 bp, Artificial Sequence
  Description of Artificial Sequence Synthetic oligonucleotide
cugcuggaccguggcgccatt

SEQ ID NO: 752 - DNA, 21 bp, Artificial Sequence
  Description of Artificial Sequence Synthetic oligonucleotide
uggcgccacgguccagcagtt

SEQ ID NO: 753 - DNA, 21 bp, Artificial Sequence
  Description of Artificial Sequence Synthetic oligonucleotide
acaguugaauauccaugugtt

SEQ ID NO: 754 - DNA, 21 bp, Artificial Sequence
  Description of Artificial Sequence Synthetic oligonucleotide
cacauggauauucaacugutt

SEQ ID NO: 755 - DNA, 21 bp, Artificial Sequence
  Description of Artificial Sequence Synthetic oligonucleotide
gcugcuggaccguggcgcctt

SEQ ID NO: 756 - DNA, 21 bp, Artificial Sequence
  Description of Artificial Sequence Synthetic oligonucleotide
ggcgccacgguccagcagctt

SEQ ID NO: 757 - DNA, 21 bp, Artificial Sequence
  Description of Artificial Sequence Synthetic oligonucleotide
gcugcuggaccguggcgcctt

SEQ ID NO: 758 - DNA, 21 bp, Artificial Sequence
  Description of Artificial Sequence Synthetic oligonucleotide
ggcgccacgguccagcagctt

SEQ ID NO: 759 - DNA, 21 bp, Artificial Sequence
  Description of Artificial Sequence Synthetic oligonucleotide
cugcuggaccguggcgccatt

SEQ ID NO: 760 - DNA, 21 bp, Artificial Sequence
  Description of Artificial Sequence Synthetic oligonucleotide
uggcgccacgguccagcagtt

SEQ ID NO: 761 - DNA, 20 bp, Artificial Sequence
  Description of Artificial Sequence Synthetic oligonucleotide
tgtccgcaactacaacgcct